SEQUENCE LISTING (8 sequences)

SEQ ID NO: 1 - protein, 559 aa, Artificial Sequence
  Description of Artificial Sequence Synthetic polypeptide
MKWVTFISLLFLFSSAYSRGVFRRRAEEPAVGTSGLIFREDLDWPPGSPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRARRSAGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR

SEQ ID NO: 2 - DNA, 2065 bp, Homo sapiens
gcatgttgacacatcaggcccagctctatcactggggagggagataggctgccagggacagaaagggctctttgagaaggccactctgcctggagtgggggcgccgggcactgtcccccaaggtcgcggcagaggagataggggtctgtcctgcacaaacaccccaccttccactcggctcacttaaggcaggcagcccagcccctggcagcacccacgatgcgggacctgcctctcaccagcctggccctagtgctgtctgccctgggggctctgctggggactgaggccctcagagcagaggagccagctgtgggcaccagtggcctcatcttccgagaagacttggactggcctccaggcagcccacaagagcctctgtgcctggtggcactgggcggggacagcaatggcagcagctcccccctgcgggtggtgggggctctaagcgcctatgagcaggccttcctgggggccgtgcagagggcccgctggggcccccgagacctggccaccttcggggtctgcaacaccggtgacaggcaggctgccttgccctctctacggcggctgggggcctggctgcgggaccctggggggcagcgcctggtggtcctacacctggaggaagtgacctgggagccaacaccctcgctgaggttccaggagcccccgcctggaggagctggccccccagagctggcgctgctggtgctgtaccctgggcctggccctgaggtcactgtgacgagggctgggctgccgggtgcccagagcctctgcccctcccgagacacccgctacctggtgttagcggtggaccgccctgcgggggcctggcgcggctccgggctggccttgaccctgcagccccgcggagaggactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggccatccgcggaactcgaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgcccaggtggccccggcggcctcggcgatcccctgcgagcgctgctgctcctgaaggcgctgcagggcctgcgcgtggagtggcgcgggcgggatccgcgcgggccgggtcgggcacagcgcagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggagcgcatcagcgcgcaccacgtgcccaacatggtggccaccgagtgtggctgccggtgacccctgcgccgcgcggactcctgccccgagggtccggacgcgccccagctcgcgccccttcccatatttattcggaccccaagcatcgccccaataaagaccagcaagcaaccggcaaaaaaaaaaaaaaaaaaaaaaaaaaaaaaaaaaaaaaaaaaaaaaa

SEQ ID NO: 3 - DNA, 3100 bp, Homo sapiens
cacatcaggcccagctctatcactggggagggagataggctgccagggacagaaagggctctttgagaaggccactctgcctggagtgggggcgccgggcactgtcccccaaggtcgcggcagaggagataggggtctgtcctgcacaaacaccccaccttccactcggctcacttaaggcaggcagcccagcccctggcagcacccacgatgcgggacctgcctctcaccagcctggccctagtgctgtctgccctgggggctctgctggggactgaggccctcagagcagaggagccagctgtgggcaccagtggcctcatcttccgagaagacttggactggcctccaggcatcccacaagagcctctgtgcctggtggcactgggcggggacagcaatggcagcagctcccccctgcgggtggtgggggctctaagcgcctatgagcaggccttcctgggggccgtgcagagggcccgctggggcccccgagacctggccaccttcggggtctgcaacaccggtgacaggcaggctgccttgccctctctacggcggctgggggcctggctgcgggaccctggggggcagcgcctggtggtcctacacctggaggaaggtatgtggggcccagccccaagcttggcaccgccgtcttccttcaggtgggccgggtcctcctagggaagatcaggggctggcagagcccccaccctgggcagggaggctgtggtcttgttcctaggactgggttgcgggtccgtggcctggaaggtgggcaccacactctgtcctgtccccgaagcccagctcttagacttgcccctgcctcggtgccagggagagagctgctgccttctccccacccctgaagacgacgcagggctcggggccagtggaacccttcttcccacagccccagcctgttctcagggccgctggcctaagatactccctgcggggaaggggcttcatcgggcaccccaacccagagaccccagggcggcagccccacccacagcctcagacgcagcccctgcctgcccctgccgtcaccgctccctggctgcaggaaggcagctaagaggggcacccttgtcccccgcttgaggtcccctgcacagtggccagagcggcagggacagatcccaaagattcccggggggtgtggccttcaatggctcaggcgtcccctgctgtcccggctgcagtgacctgggagccaacaccctcgctgaggttccaggagcccccgcctggaggagctggccccccagagctggcgctgctggtgctgtaccctgggcctggccctgaggtcactgtgacgagggctgggctgccgggtgcccaggtaccagggagttgcatggggcagtgcccgggccgtggcggggggcatgaatttgttgcagggtctgcagtactgagaacagcgtagaaccagtggcgatgggaggaaggggaccggtagagcggggctgggtaagcctccatccagccgggctgagccctggtctccgcagagcctctgcccctcccgagacacccgctacctggtgttagcggtggaccgccctgcgggggcctggcgcggctccgggctggccttgaccctgcagccccgcggagagggtaggtccgcgtggagagggacggggagccgggtcgactgcccccgggcccccagcccctgagccagccgcgtgcccacccaccgcagactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggtgcgcgcaggcaccgggacacggggcaggagcgggcgggggcggcgtggcctcgtggccgctctcaactcctccaattgcgggttccaggccatccgcggaactcgaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgcccaggaggccccggcggcctcggcgatcccctgcgagcgctgctgctcctgaaggcgctgcagggcctgcgcgtggagtggcgcgggcgggatccgcgcgggccgggtcgggcacagcgcagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggaacgcatcagcgcgcaccacgtgcccaacatggtggccaccgagtgtggctgccggtgacccctgcgccgcgcggactcctgccccgagggtccggacgcgccccagctcgcgccccttcccatatttattcggaccccaagcatcgccccaataaagaccagcaagcaaccggctggggtgtccgtgcgtgttagggggcccgtgggacctcccttgccgtctctcctcgcgcacggcccgggtccgccctgtagcgctcgctgtctctcccctgcctgaagcgccccaccaccgtctttcaggccccggacttggtgccggg

SEQ ID NO: 4 - DNA, 1855 bp, Homo sapiens
atctgaagaaagatttggccaggggcagctgtgctggcttatgctcttctccttctgctgctgccatcctccagcaagatgctagggtctttggggctttgggcattacttcccacagctgtggaagcacccccaaacaggcgaacctgtgtgttctttgaggcccctggagtgcggggaagcacaaagacactgggagagctgctagatacaggcacagagctccccagagctatccgctgcctctacagccgctgctgctttgggatctggaacctgacccaagaccgggcacaggtggaaatgcaaggatgccgagacagtgatgagccaggctgtgagtccctccactgtgacccaagtccccgagcccaccccagccctggctccactctcttcacctgctcctgtggcactgacttctgcaatgccaattacagccatctgcctcctccagggagccctgggactcctggctcccagggtccccaggctgccccaggtgagtccatctggatggcactggtgctgctggggctgttcctcctcctcctgctgctgctgggcagcatcatcttggccctgctacagcgaaagaactacagagtgcgaggtgagccagtgccagagccaaggccagactcaggcagggactggagtgtggagctgcaggagctgcctgagctgtgtttctcccaggtaatccgggaaggaggtcatgcagtggtttgggccgggcagctgcaaggaaaactggttgccatcaaggccttcccaccgaggtctgtggctcagttccaagctgagagagcattgtacgaacttccaggcctacagcacgaccacattgtccgatttatcactgccagccgggggggtcctggccgcctgctctctgggcccctgctggtactggaactgcatcccaagggctccctgtgccactacttgacccagtacaccagtgactggggaagttccctgcggatggcactgtccctggcccagggcctggcatttctccatgaggagcgctggcagaatggccaatataaaccaggtattgcccaccgagatctgagcagccagaatgtgctcattcgggaagatggatcgtgtgccattggagacctgggccttgccttggtgctccctggcctcactcagccccctgcctggacccctactcaaccacaaggcccagctgccatcatggaagctggcacccagaggtacatggcaccagagctcttggacaagactctggacctacaggattggggcatggccctccgacgagctgatatttactctttggctctgctcctgtgggagatactgagccgctgcccagatttgaggcctgacagcagtccaccacccttccaactggcctatgaggcagaactgggcaatacccctacctctgatgagctatgggccttggcagtgcaggagaggaggcgtccctacatcccatccacctggcgctgctttgccacagaccctgatgggctgagggagctcctagaagactgttgggatgcagacccagaagcacggctgacagctgagtgtgtacagcagcgcctggctgccttggcccatcctcaagagagccacccctttccagagagctgtccacgtggctgcccacctctctgcccagaagactgtacttcaattcctgcccctaccatcctcccctgtaggcctcagcggagtgcctgccacttcagcgttcagcaaggcccttgttccaggaatcctcagcctgcctgtaccctttctcctgtgtaaatatgcagtttatgtgtcatcaatgtacatgccaacataaatatggcgattgtat

SEQ ID NO: 5 - DNA, 1843 bp, Homo sapiens
ggcacgagggcagctgtgctggcttatgctcttctccttctgctgctgccatcctccagcaagatgctagggtctttggggctttgggcattacttcccacagctgtggaagcacccccaaacaggcgaacctgtgtgttctttgaggcccctggagtgcggggaagcacaaagacactgggagagctgctagatacaggcacagagctccccagagctatccgctgcctctacagccgctgctgctttgggatctggaacctgacccaagaccgggcacaggtggaaatgcaaggatgccgagacagtgatgagccaggctgtgagtccctccactgtgacccaagtccccgagcccaccccagccctggctccactctcttcacctgctcctgtggcactgacttctgcaatgccaattacagccatctgcctcctccagggagccctgggactcctggctcccagggtccccaggctgccccaggtgagtccatctggatggcactggtgctgctggggctgttcctcctcctcctgctgctgctgggcagcatcatcttggccctgctacagcgaaagaactacagagtgcgaggtgagccagtgccagagccaaggccagactcaggcagggactggagtgtggagctgcaggagctgcctgagctgtgtttctcccaggtaatccgggaaggaggtcatgcagtggtttgggccgggcagctgcaaggaaaactggttgccatcaaggccttcccaccgaggtctgtggctcagttccaagctgagagagcattgtacgaacttccaggcctacagcacgaccacattgtccgatttatcactgccagccgggggggtcctggccgcctgctctctgggcccctgctggtactggaactgcatcccaagggctccctgtgccactacttgacccagtacaccagtgactggggaagttccctgcggatggcactgtccctggcccagggcctggcatttctccatgaggagcgctggcagaatggccaatataaaccaggtattgcccaccgagatctgagcagccagaatgtgctcattcgggaagatggatcatgtgccattggagacctgggccttgccttggtgctccctggcctcactcagccccctgcctggacccctactcaaccacaaggcccagctgccatcatggaagctggcacccagaggtacatggcaccagagctcttggacaagactctggacctacaggattggggcatggccctccgacgagctgatatttactctttggctctgctcctgtgggagatactgagccgctgcccagatttgaggcctgacagcagtccaccacccttccaactggcctatgaggcagaactgggcaatacccctacctctgatgagctatgggccttggcagtgcaggagaggaggcgtccctacatcccatccacctggcgctgctttgccacagaccctgatgggctgagggagctcctagaagactgttgggatgcagacccagaagcacggctgacagctgagtgtgtacagcagcgcctggctgccttggcccatcctcaagagagccacccctttccagagagctgtccacgtggctgcccacctctctgcccagaagactgtacttcaattcctgcccctaccatcctcccctgtaggcctcagcggagtgcctgccacttcagcgttcagcaaggcccttgttccaggaatcctcagcctgcctgtaccctttctcctgtgtaaatatgcagtttatgtatcaatgtacatgccaacataaatatggcgattgtatagctgt

SEQ ID NO: 6 - DNA, 1680 bp, Artificial Sequence
  Description of Artificial Sequence Synthetic polynucleotide
atgaagtgggtgagcttcatcagcctgctgttcctgttcagcagcgcttactcccgcggtgtgttccgccgcagagcagaggagccagctgtgggcaccagtggcctcatcttccgagaagacttggactggcctccaggcagcccacaagagcctctgtgcctggtggcactgggcggggacagcaatggcagcagctcccccctgcgggtggtgggggctctaagcgcctatgagcaggccttcctgggggccgtgcagagggcccgctggggcccccgagacctggccaccttcggggtctgcaacaccggtgacaggcaggctgccttgccctctctacggcggctgggggcctggctgcgggaccctggggggcagcgcctggtggtcctacacctggaggaagtgacctgggagccaacaccctcgctgaggttccaggagcccccgcctggaggagctggccccccagagctggcgctgctggtgctgtaccctgggcctggccctgaggtcactgtgacgagggctgggctgccgggtgcccagagcctctgcccctcccgagacacccgctacctggtgttagcggtggaccgccctgcgggggcctggcgcggctccgggctggccttgaccctgcagccccgcggagaggactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggccatccgcggaactcgaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgcccaggtggccccggcggcctcggcgatcccctgcgagcgctgctgctcctgaaggcgctgcagggcctgcgcgtggagtggcgcgggcgggatccgcgcgggccgggtcgggcacggcgcagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggagcgcatcagcgcgcaccacgtgcccaacatggtggccaccgagtgtggctgccggtga

SEQ ID NO: 7 - DNA, 49 bp, Artificial Sequence
  Description of Artificial Sequence Synthetic oligonucleotide
aataggtgattttggtctagctacagagaaatctcgatggagtgggtcc

SEQ ID NO: 8 - protein, 13 aa, Artificial Sequence
  Description of Artificial Sequence Synthetic peptide
FWSSYREISMEWV

(19) United States
(12) Patent Application Publication
(10) Pub. No.: US 2016/0250190 A1
(43) Pub. Date: Sep. 1, 2016

(54) N-METHYL PYRAZOLOANTHRONE FOR TREATMENT OF CANCER

(71) Applicant: THE GENERAL HOSPITAL CORPORATION, Boston, MA (US)

(72) Inventors: David PEPIN, Somerville, MA (US); Patricia K. DONAHOE, Boston, MA (US)

(73) Assignee: THE GENERAL HOSPITAL CORPORATION, Boston, MA (US)

(21) Appl. No.: 15/029,402

(22) PCT Filed: Oct. 17, 2014
(86) PCT No.: PCT/US2014/061085

§ 371 Date: Apr. 14, 2016

Related U.S. Application Data
(60) Provisional application No. 61/893,065, filed on Oct. 18, 2013.

(51) Int. Cl. A61K 31/4184; A61K 33/24; A61K 31/704; A61K 45/06

(52) U.S. Cl. CPC A61K 31/4184; A61K 45/06; A61K 33/24; A61K 31/704

(57) ABSTRACT

The present invention relates to a N1-methyl-pyrazoloanthrone, e.g., a N1-methyl 1,9-pyrazoloanthrone or functional derivatives or analogues thereof to inhibit at least one kinase of the CK1 family (e.g., CSNK1A1, CSNK1B, CSNK1G1, CSNK1G2, CSNK1G3, CSNK1D, CSNK1E) and/or ARK5/NUAK1 in a cell. Another aspect relates to administration of N1-methyl-pyrazoloanthrone, e.g., a N1-methyl 1,9-pyrazoloanthrone or a functional derivative thereof in a method to treat cancer, e.g., a cancer with increased expression and/or a genetic alteration in at least one member of the CK1 family and/or ARK5/NUAK. Another aspect of the present invention relates to methods to decrease the dose of a chemotherapeutic agent by administering the chemotherapeutic agent in combination with a N1-methyl-pyrazoloanthrone, e.g., a N1-methyl 1,9-pyrazoloanthrone or a functional derivative or analogue thereof.

### CROSS REFERENCE TO RELATED APPLICATIONS

This application claims benefit under 35 U.S.C. 119(e) of U.S. Provisional Patent Application Ser. No. 61/893,065, filed on Oct. 18, 2013, the contents of which is incorporated herein in its entirety by reference.

### FIELD OF THE INVENTION

This application relates to compositions, methods and kits comprising n-methyl pyrazolothrone and derivatives thereof for the treatment of diseases and conditions where activation of members of the casein kinase 1 (CK1) family, ARK5/NUAK1 or the type II receptor for MIS (MISRII) is beneficial, including but not limited to, cancers and disorders associated with excess androgen states.

### SEQUENCE LISTING

The instant application contains a Sequence Listing which has been submitted electronically in ASCII format and is hereby incorporated by reference in its entirety. Said ASCII copy, created on Oct. 16, 2014, is named 030258-078401-PCT_SL.txt and is 20,087 bytes in size.

### BACKGROUND OF THE INVENTION

Cancer is characterized by uncontrolled growth, proliferation, and migration of cells. Cancer is the second leading cause of death with 500,000 deaths and an estimated 1.3 million new cases in the United States in 1996. The role of signal transduction pathways contributing to cell transformation and cancer is a generally accepted concept.

Müllerian Inhibiting Substance (MIS) is a glycoprotein hormone secreted by the newly differentiating testis during the fetal period where it is responsible for regression of the Müllerian ducts in males, which would otherwise develop into the internal female reproductive tract tissues. After MIS binds to the type II receptor for MIS (MISRII), it recruits, phosphorylates, and activates one of three possible, activin-like kinase type I receptors (Alk2 or 3), which in turn activates the Smad 1/5/8 pathway in concert with the common Smad4. The Smad complex will translocate into the nucleus and bind to promoter regions to activate transcription of MIS-responsive genes.

The continuous production of MIS in both male and females after birth indicates MIS has a function in the adult. In particular, it has been reported that postnatal expression of MIS functions as an inhibitor of tumor cell proliferation, such as cancers expressing MISRII, including, but not limited to breast (Gupta, 2005, Segev, 2000), prostate (Hoshiya, 2003, Tran, 2006), cervical (Barbie, 2003), endometrial (Renaud, 2005) and ovarian (Ha, 2000, Stephen, 2002) cancers.

However, the production of large quantities of purified, biologically active MIS sufficient to be used therapeutically in the treatment of cancers and other disorders where it is desirable to activate MISRII is challenging. Therefore, it would be desirable to have a simple molecule that could mimic the effect of MIS by selectively activating the MISRII-mediated downstream signaling pathway.

Previously, the inventors demonstrated that the molecule SP600125 (also known as SP600) was shown as a potent activator of MISRII in a small molecule screen of MIS mimetics (Renlund et al. 2008). Additionally, SP600 was shown that, like MIS, to be effective in inhibiting a putative cancer stem cell population in ovarian cancer (Wei et al. 2010).

SP600125 is an anthrapyrazolone small molecule originally developed by Celgene as a reversible ATP-competitive inhibitor of JNK 1, 2, and 3 (Bennett et al. 2001). The ability of SP600 to inhibit c-Jun phosphorylation and its sequela, the induction of cytokines such as TNF-α, IFN-γ, and IL-2 made it an attractive candidate therapeutic for the treatment of inflammatory diseases such as arthritis. In a rat model of arthritis, SP600 moderately reduced paw swelling, but importantly almost completely prevented joint destruction as imaged by radiography (Han et al. 2001). Interestingly, anthrapyrazolones have long been part of the pharmacopeia for the treatment of cancer due to their DNA-intercalating properties (Showalter et al. 1987).

SP600, however, does not bind to DNA because it lacks the long branching chains characteristics of other pyrazolone cancer drugs such as Doxorubicin. It was believed that SP600 might harbor anti-cancer properties from its ability to inhibit the JNK pathway. JNKs are members of the MAPK pathway which has been implicated in many aspects of cancer biology such as proliferation, apoptosis, adhesion, migration, differentiation and tumorigenesis. Furthermore, c-Jun, a canonical target of JNKs, is a bona-fide proto-oncogene and is involved in the cell's response to UV damage. As such SP600 was tested under the hypothesis that its inhibition of the JNK pathway would result in inhibition of tumor growth (Ennis et al. 2004). Remarkably SP600 inhibited both cancer cells and endothelial cell growth and migration in-vitro, in part by inducing a G2-M block in the cell cycle (Ennis et al. 2004). Additionally, when given intra-peritoneally in a nude mouse model of cancer, it was effective at slowing tumor growth using prostate and lung cancer cell lines either alone or in combination with other chemotherapeutics (Ennis et al. 2004). It has also been proposed that the ability of SP600125 to inhibit JNKs in the cancer stem cell population may underlie its effectiveness against other xenograft models such as glioblastoma (Matsuda et al. 2012).

### SUMMARY OF THE INVENTION

The inventors have discovered that N-methyl pyrazoloathrone or derivatives thereof can inhibit the growth of a cancer cell which expresses at least one kinase of the Casein Kinase I family (e.g., CSNK1A1, CSNK1B, CSNK1G1, CSNK1G2, CSNK1G3, CSNK1D, CSNK1E) and/or ARK5/NUAK1. The present invention relates to the use of N-methyl pyrazoloathrone, in particular, N1-methyl pyrazoloathrone or derivatives thereof to inhibit at least one kinase of from the Casein Kinase I family (e.g., CSNK1A1, CSNK1B, CSNK1G1, CSNK1G2, CSNK1G3, CSNK1D, CSNK1E) and/or ARK5/NUAK1 where at least one downstream effect is the inhibition of tumor cell proliferation, for example inhibition of cancers expressing, or having at least one genetic alteration (e.g., mutation and/or SNP) in at least one kinase of the Casein Kinase I family (e.g., CSNK1A1, CSNK1B, CSNK1G1, CSNK1G2, CSNK1G3, CSNK1D, CSNK1E) and/or ARK5/NUAK1. In some embodiments, a cancer which expresses at least one kinase of the Casein Kinase I family (e.g., CSNK1A1, CSNK1B, CSNK1G1, CSNK1G2, CSNK1G3, CSNK1D, CSNK1E) and/or ARK5/NUAK1 is ovarian cancer.

The present invention is based on the discovery of N1-methyl-1,9,-pyrazoloathrone (also referred to herein as M-SP600), which is a derivative of 1,9-pyrazoloathrone, known as SP600125 (also referred herein to as “SP600”) (CAS#129-56-6) can inhibit the proliferation of cancer cells. SP600 is recognized an anti-cancer therapeutic functioning by inhibiting Jun kinases (e.g., JNK1, JNK2, JNK3). The inventors have surprisingly discovered that the addition of a methyl group to a nitrogen, specifically, the first nitrogen of the tri-cyclic structure to produce N1-methyl-1,9,-pyrazoloathrone (CAS #54642-23-8) results in killing cancer cells, and unexpectedly discovered that N1-methyl-1,9,-pyrazoloathrone functions to kill cancer cells or reduce their rate of proliferation via a JNK-independent mechanism. As SP600 kills cancer cells via inhibition of JNK kinases, it was particularly unexpected that the N1-methyl modification abrogated the inhibition of JNKs, yet conserved the killing or anti-cancer activity of the compound. Accordingly, the inventors have surprisingly discovered that, unlike its analogue 1,9-pyrazoloathrone (SP-600125), N1-methyl-1,9,-pyrazoloathrone functions as an anti-cancer agent not through inhibition of Jun kinases (JNK1, JNK2 or JNK3), but rather, via inhibition of at least one member of the Casein Kinase I family (e.g., CSNK1A1, CSNK1B, CSNK1G1, CSNK1G2, CSNK1G3, CSNK1D, CSNK1E) and/or ARK5/NUAK1.

Furthermore, the inventors demonstrate that the N1-methyl-1,9-pyrazolothrone has much fewer off-target effects than SP600125, demonstrating a better toxicity profile at specifically targeting the death of cancer cells. For example, using the Kinase Inhibitor Resource of the Fox Chase Cancer Center list, the inventors demonstrate that 0.5 μM of SP60012534 inhibited over 50% of the 34 kinases, whereas N1-methyl-1,9-pyrazolothrone (at the same concentration) only inhibited 4 of the 34 kinases. Furthermore, the inventors demonstrate that N1-methyl-1,9-pyrazolothrone inhibits Casein Kinase I family (e.g., CSNK1A1, CSNK1B, CSNK1G1, CSNK1G2, CSNK1G3, CSNK1D, CSNK1E) and/or ARK5/NUAK1 expressing cancer cells, such as for example ovarian cancer cells, and is highly synergistic with other chemotherapeutic drugs, such as, for example, cisplatin, doxorubicin and paclitaxel, and targeted therapeutics such as, for example, vermurafib, which is currently approved for the treatment of cancer. Additionally, the inventors have discovered that N1-methyl-1,9-pyrazolothrone is also synergistic with MIS (including recombinant human MIS protein (rhMIS) or MIS variants, e.g., LR-MIS as disclosed herein) in inhibiting cancer cells, including but not limited to cancer stem cells such as, for example, ovarian cancer stem cells.

Accordingly, the inventors have surprisingly discovered that a chemical modification of a methyl group at position N1 on 1,9-pyrazolothrone to render N1-methyl-1,9-pyrazolothrone, changes the activity profile of the compound. The inventors have also surprisingly discovered that N1-methyl-1,9-pyrazolothrone does not target and kill cancer cells by inhibiting JNKs, but rather by acting in a JNK-independent mechanism which causes cell cycle arrest. The inventors demonstrate that N1-methyl-1,9-pyrazolothrone primarily inhibits the Casein Kinase I family (e.g., CSNK1A1, CSNK1B, CSNK1G1, CSNK1G2, CSNK1G3, CSNK1D, CSNK1E) and ARK5/NUAK1. Importantly, the inventors also demonstrate that N1-methyl-1,9-pyrazolothrone has fewer unwanted off-target effects on other kinases as compared to SP600125.

Accordingly, because N1-methyl-1,9-pyrazolothrone can act synergistically with other chemotherapeutic agents, N1-methyl-1,9-pyrazolothrone can be used in combination with such other chemotherapeutic agents to lower the effective concentration of MIS and/or other chemotherapeutic agents in the treatment of cancers expressing at least one member of the Casein Kinase I family (e.g., CSNK1A1, CSNK1B, CSNK1G1, CSNK1G2, CSNK1G3, CSNK1D, CSNK1E) and/or ARK5/NUAK1 and/or MIS receptors, for example cancer expressing MISRII. Examples of such agents are, for example but not limited to, chemotherapy agents such as cisplatin, doxorubicin and paclitaxel, vermurafib, as well as MIS and recombinant forms of MIS (e.g., recombinant human MIS or rhMIS). In some embodiments, N1-methyl-1,9-pyrazolothrone can be used in compositions, e.g., in methods for the treatment of cancers expressing at least one member of the Casein Kinase I family (e.g., CSNK1A1, CSNK1B, CSNK1G1, CSNK1G2, CSNK1G3, CSNK1D, CSNK1E) and/or ARK5/NUAK1 and/or expressing MIS receptors, for example cancer expressing MISRII. Such cancers expressing at least one member of the Casein Kinase I family (e.g., CSNK1A1, CSNK1B, CSNK1G1, CSNK1G2, CSNK1G3, CSNK1D, CSNK1E) and/or ARK5/NUAK1 include, for example, ovarian cancer, cervical cancer, breast cancer, prostate cancer, and endometrial cancer.

N1-methyl-1,9-pyrazoloanthrone (M-SP600) was initially reported in the original study which led to the development of SP600 (Bennet et al. 2001). M-SP600 differs only from SP600125 by an additional methyl group positioned on the N of the pyrazole ring. The presence of the free NH on the pyrazole ring is thought to be required for hydrogen bonding at the ATP-binding site of JNKs, and as such MSP600 does not inhibit JNKs (Bennet et al. 2001). In fact, M-SP600 is commercially available as a negative control for SP600, and has been used as such in several cancer studies (Haga et al. 2009) (Haga et al 2008). In contrast to previous reports, where M-SP600 was shown not to kill cancer cells, the inventors have surprisingly demonstrated that M-SP600 can inhibit cancer cell proliferation similar to that of SP600, despite not inhibiting JNK or the phosphorylation of c-Jun. Rather, the inventors have demonstrated herein that M-SP600 functions to induce cell cycle arrest in cancer cells via a JNK-independent mechanism, thereby killing cancer cells and inhibiting cancer cell proliferation via a novel mechanism independent of JNK. Furthermore, the inventors demonstrate herein that MSP600 treatment induces a G1 arrest, and results in upregulation of P21 and P15. Finally gene expression data and a broad kinase screen demonstrate that MSP600 inhibits the casein kinase I (CK1) family. Therefore, MSP600 is useful herein as a potent cancer therapeutic which is vastly improved over SP600 by its more narrow kinase inhibition profile, and its lack of inhibition of the immune system, which is increasingly recognized as an important component of treatment success.

Accordingly, one aspect of the present invention relates to compositions comprising a N-methyl pyrazoloathrone, such as N1-methyl pyrazoloathrone in methods for the treatment of cancers, for example ovarian cancer and other cancer expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1. In some embodiments, the subject is at risk of developing, or has a cancer, e.g., ovarian cancer, or a cancer expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1. In some embodiments, a subject amenable to treatment with the N1-methyl-1,9-pyrazolothrone is at risk of developing, or has a cancer, such as ovarian cancer or a cancer expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1.

In some embodiments, a subject has a cancer with at least one genetic alteration in at least one member of the casein kinase 1 family (e.g., a mutation or SNP in at least one of the following genes, CSNK1A1, CSNK1B, CSNK1D, CSNK1E, CSNK1G1, CSNK1G2, CSNK1G3). 18.3% of subjects with a genetic alteration (e.g., SNP or or genetic mutation) in a casein kinase gene can develop ovarian cancer (see FIG. 10 herein). Accordingly, in some embodiments, a subject treated with a a compound of formula (I)-(VII), such as N1-methyl 1,9-pyrazoloanthrone as disclosed herein has at least one genetic alteration in at least one member of the casein kinase 1 family (e.g., a mutation or SNP in at least one of the following genes, CSNK1A1, CSNK1B, CSNK1D, CSNK1E, CSNK1G1, CSNK1G2, CSNK1G3). In such embodiments, such a subject can have at least one of the following cancers: ovarian cancer, prostate cancer, bladder cancer, melanoma, pancreatic cancer, sarcoma, liver cancer, stomach cancer, breast cancer, uterine cancer or adenoid cancer.

In some embodiments, a subject treated with a a compound of formula (I)-(VII), such as N1-methyl 1,9-pyrazoloanthrone as disclosed herein has at least one genetic alteration (e.g., a SNP or or genetic mutation) in ARK3/NUAK1. In such embodiments, such a subject can have at least one of the following cancers: pancreatic cancer, melanoma, prostate cancer, sarcoma, stomach cancer, lung cancer, uterine cancer, colorectal cancer, colon cancer, esophageal cancer and/or bladder cancer.

In some embodiments, a biological sample is harvested from a subject with cancer to determine if the cancer expresses at least one kinase of the Casein Kinase I (CKI) family (e.g., CSNK1A1, CSNK1B, CSNK1G1, CSNK1G2, CSNK1G3, CSNK1D, CSNK1E) and/or ARK5/NUAK1, or if the subject has at least one genetic alteration (e.g. mutation or SNP) in ARK5/NUAK1 gene and/or at least one member of the casein kinase 1 family (e.g., a mutation or SNP in at least one of the following genes, CSNK1A1, CSNK1B, CSNK1D, CSNK1E, CSNK1G1, CSNK1G2, CSNK1G3). In some embodiments, the biological sample is a tissue sample, for example a cancer or tumor tissue sample or a cancer cell or tumor cell, or a biopsy tissue sample. In some embodiments, where the sample is being tested for a genetic alteration in a member of the CK1 family and/or ARK5/NUAK1, the biological sample is blood, saliva, plasma, or urine or any sample obtained from the subject comprising the subjects cells.

One aspect of the present invention provides methods to treat cancer, such as, for example, ovarian cancer and cancers expressing, or having at least one genetic alteration (e.g., mutation and/or SNP) in at least one kinase of the Casein Kinase I family (e.g., CSNK1A1, CSNK1B, CSNK1G1, CSNK1G2, CSNK1G3, CSNK1D, CSNK1E) and/or ARK5/NUAK1 and/or a MIS receptor, for example cancer expressing MISRII by administering to the subject a pharmaceutical composition comprising a N-methyl pyrazoloathrone, such as N1-methyl pyrazoloathrone or the N1-methyl-1,9-pyrazolothrone or a derivative or analogue thereof. The present invention relates to a method to treat cancers, including but not limited to ovarian cancer, and other cancers expressing, or having at least one genetic alteration (e.g., mutation and/or SNP) in at least one kinase of the Casein Kinase I family (e.g., CSNK1A1, CSNK1B, CSNK1G1, CSNK1G2, CSNK1G3, CSNK1D, CSNK1E) and/or ARK5/NUAK1 and/or a MIS receptor, for example cancer expressing MISRII, the method comprising contacting a cell with a N-methyl pyrazoloathrone, such as N1-methyl-1,9-pyrazolothrone or a derivative or analogue thereof. In some embodiments N-methyl pyrazoloathrone is N1-methyl-1,9-pyrazolothrone or an analogue or derivative thereof. In some embodiments, the cell, for example a cancer cell which expresses at least one kinase of the Casein Kinase I family (e.g., CSNK1A1, CSNK1B, CSNK1G1, CSNK1G2, CSNK1G3, CSNK1D, CSNK1E) and/or ARK5/NUAK1 is also contacted with agents in addition to a N-methyl pyrazoloathrone, such as N1-methyl pyrazoloathrone or derivative or analogue thereof, and in some embodiments, the agents are therapeutic agents and/or chemotherapeutic agents. The cell expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1 and/or a MIS receptor or MISRII can be any cancer cell, including but not limited to cancer stem cells. In some embodiments, the cell is a cancer cell expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1. In some embodiments, the cell is a cancer stem cell expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1. In some embodiments, the cell is an ovarian cancer cell or an ovarian cancer stem cell expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1.

In another embodiment, a biological sample is obtained from the subject with cancer and assessed for the expression of at least one kinase of the Casein Kinase I family (e.g., CSNK1A1, CSNK1B, CSNK1G1, CSNK1G2, CSNK1G3, CSNK1D, CSNK1E) and/or ARK5/NUAK1. Optionally, the biological sample is also assessed for the expression of a MIS receptor, for example for the expression of MISRII. If the presence of expression and/or activity of at least one kinase of the Casein Kinase I family (e.g., CSNK1A1, CSNK1B, CSNK1G1, CSNK1G2, CSNK1G3, CSNK1D, CSNK1E) and/or ARK5/NUAK1 and/or a MIS receptor (e.g., MISRII) is detected, the subject is administered a pharmaceutical composition comprising a N-methyl pyrazoloathrone, such as N1-methyl pyrazoloathrone or a derivative or analogue thereof. In another embodiment, the pharmaceutical composition comprising a N-methyl pyrazoloathrone, such as N1-methyl pyrazoloathrone comprises additional therapeutic agents and/or chemotherapeutic agents and/or MIS or hrMIS.

In another aspect of the present invention, the methods relate to the use of a N-methyl pyrazoloathrone, such as N1-methyl pyrazoloathrone and functional derivatives thereof for the treatment of any disorder where inhibition of at least one kinase of the Casein Kinase I family (e.g., CSNK1A1, CSNK1B, CSNK1G1, CSNK1G2, CSNK1G3, CSNK1D, CSNK1E) and/or ARK5/NUAK1 is whole, or part, of the therapeutic regime.

In some embodiments, the N1-methyl-pyrazolothrone is N1-methyl-1,9-pyrazolothrone, referred herein to as “M-SP600” or “M-SP600125” having the following structure.

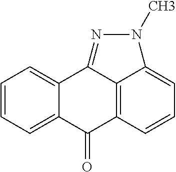 [SMILES: Cn1nc2c3c(cccc31)C(=O)c1ccccc1-2]

In some embodiments, the N1-methyl-pyrazoloanthrone, is N1-methyl-1,9-pyrazolothrone or a functional derivative of N1-methyl-1,9-pyrazolothrone, and may generally be classified as “N1-methyl-1,9-pyrazolothrone derivatives” having the following structure (II), also referred to herein as Compound (II):

In some embodiments, the compounds of this invention have the following structure (I):

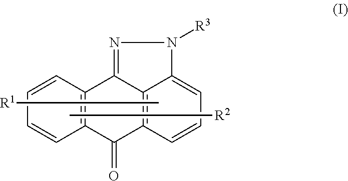

### Wherein:

R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶; R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and pharmaceutically acceptable salts thereof.

In some embodiments, R¹ and R² are both absent, i.e., compounds are of structure (II):

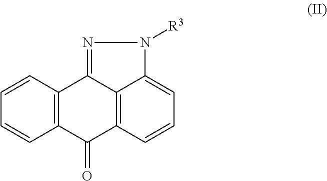

In some embodiment, only one of R¹ and R² is present, i.e., compounds are of structure (III) or (IV):

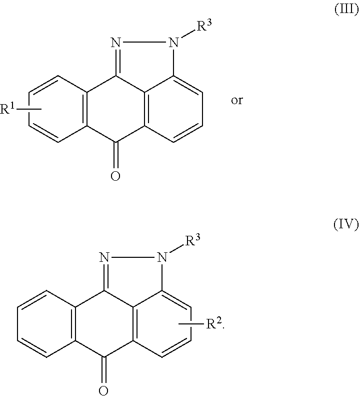

In some other embodiments, R¹ and R² are both present and are attached to the same ring, i.e., compounds of structure (V) or (VI):

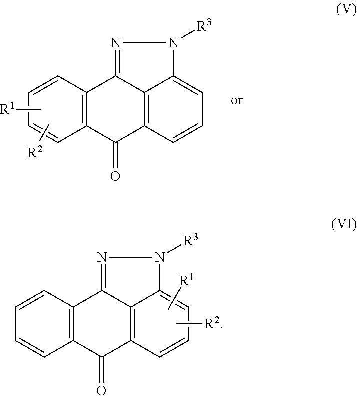

In some embodiments, R¹ and R² are both present and are attached to the different rings, i.e., compounds of structure (VII):

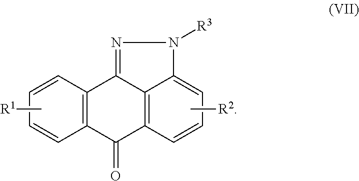

In embodiments of the compounds disclosed herein, R³ can be a C₁-C₆ alkyl. Exemplary C₁-C₆ alkyl include, but are not limited to, methyl, ethyl, n-propyl, isopropyl, 1-methyl-propyl, n-butyl, isobutyl, t-butyl, 1-methyl-butyl, pentyl, hexyl, propylenyl, 1-butenyl, propynyl, and the like. In one embodiment, R³ is methyl.

In some embodiments of the compounds disclosed herein, R³ can be an alkyl wherein backbone of the alkyl is interspersed with one or more hetero groups or atoms selected from 0, NH, S, SS, SO, SO₂, and any combinations thereof.

In some embodiments of the compounds disclosed herein, R³ can be a substituted alkyl.

The compounds disclosed herein can generally be made by organic synthesis techniques known to those skilled in the art, as well as by the methods disclosed in U.S. Pat. No. 7,119,114, which is incorporated herein in its entirety by reference.

In some embodiments, the disorder is a proliferative disease where the proliferative disease is associated with cells expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1. One example of such a proliferative disease is, for example, a cancer expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1. In some embodiments, the disorder is a proliferative disease where the proliferative disease is associated with at least one genetic alteration in ARK1/NUAK1 gene and/or at least one member of the casein kinase 1 family (e.g., a mutation or SNP in at least one of the following genes, CSNK1A1, CSNK1B, CSNK1D, CSNK1E, CSNK1G1, CSNK1G2, CSNK1G3). In some embodiments, the proliferative disease associated with at least one genetic alteration in at least one member of the casein kinase 1 family is selected from the group consisting of: ovarian cancer, prostate cancer, bladder cancer, melanoma, pancreatic cancer, sarcoma, liver cancer, stomach cancer, breast cancer, uterine cancer or adenoid cancer. In some embodiments, the proliferative disease associated with at least one genetic alteration in ARK1/NUAK1 gene is selected from the group consisting of: pancreatic cancer, melanoma, prostate cancer, sarcoma, stomach cancer, lung cancer, uterine cancer, colorectal cancer, colon cancer, esophageal cancer and/or bladder cancer.

In some embodiments, a cancer expressing or overexpressing (e.g., has increased expression over a pre-defined threshold level) at least one kinase of the casein kinase I family and or ARK5/NUAK1 is selected from, for example, but not limited to the following cancers, or cancers of the following tissues; brain cancer, cancer of the head and neck, renal cancer, bladder cancer, leukemia, lung cancer, melanoma, prostate cancer, cancer of the salivary gland and cancer of seminoma, lymphoblastic leukemia, colorectal cancer, hepatocellular carcinoma, colon cancer, metastatic cancer, glioma, liver cancer, NSCLC (non-small cell lung carcinoma), multiple myeloma, (see Yang et al., Genome Biology; 2008, 9: R92; Kelliher et al., EMBO J. 1996; 15(19): 5160-5166; Sinnberg et al., Cancer Res, 2010 70:17 6999-7009, Kusakai et al, Am J Pathol. 2004; 164(3):987-95, Liu et al., Nature, 2012, 483, 608-612; Kusakai et al., J. Exp. Clin. Cancer Res, 2004; 23, 2; Lu et al., Eur. J. Cancer, 2013; 49(3); 752-763; Zhu et al., In Exp Lung Res, 2013, 39(1); 9-17, which are incorporated herein in their entirety by reference.). In some embodiments, the cancer is also a MIS-responsive cancer, for example but not limited ovarian cancer and cervical cancer. In some embodiments, the cancer also expresses MISRII, for example but not limited ovarian cancer and cervical cancer.

The present invention is also directed towards methods for treating cancers expressing, or having at least one genetic alteration (e.g., mutation and/or SNP) in at least one kinase of the Casein Kinase I family (e.g., CSNK1A1, CSNK1B, CSNK1G1, CSNK1G2, CSNK1G3, CSNK1D, CSNK1E) and/or ARK5/NUAK1 by administering an effective amount of N1-methyl-1,9-pyrazolothrone or a functional derivatives thereof to a subject in need thereof. Accordingly the compounds of the present invention are useful in treatment of cancers expressing, or having at least one genetic alteration (e.g., mutation and/or SNP) in at least one kinase of the Casein Kinase I family (e.g., CSNK1A1, CSNK1B, CSNK1G1, CSNK1G2, CSNK1G3, CSNK1D, CSNK1E) and/or ARK5/NUAK1, for example ovarian cancers. The compounds of the present invention are also useful in treatment of other cancers expressing, or having at least one genetic alteration (e.g., mutation and/or SNP) in at least one kinase of the Casein Kinase I family (e.g., CSNK1A1, CSNK1B, CSNK1G1, CSNK1G2, CSNK1G3, CSNK1D, CSNK1E) and/or ARK5/NUAK1, for example cervical, breast, and prostate cancer. In some embodiments, the cancer is a cancer cell expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1. In some embodiments, the cancer cell expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1 is an ovarian cancer cell, vulvar epidermal carcinoma cell, cervical carcinoma cell, endometrial adenocarcinoma cell and ovarian adenocarcinoma.

In alternative embodiments, a cancer expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1, is for example but not limited to breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, genital-urinary cancer, stomach cancer, lymphomas, melanoma, glioma, bladder cancer, pancreatic cancer, gum cancer, kidney cancer, retinal cancer, liver cancer, nasopharynx cancer, ovarian cancer, oral cancers, bladder cancer, hematological neoplasms, follicular lymphoma, cervical cancer, multiple myeloma, osteosarcomas, thyroid cancer, prostate cancer, colon cancer, prostate cancer, skin cancer, stomach cancer, testis cancer, tongue cancer, or uterine cancer.

Accordingly, one aspect of the present invention relates to a method for treating a cancer in a subject, the method comprising administering to the subject an effective amount of a pharmaceutical composition comprising a N1-methyl-1,9-pyrazolothrone or a functional derivative or a functional analogue thereof, wherein the subject is determined to have a cancer expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1. In some embodiments, the N1-methyl-pyrazoloanthrone is N1-methyl-1,9-pyrazolothrone or functional derivative or functional analogue thereof of formula (II), and the cancer expresses at least one kinase of the Casein Kinase I family (e.g., CSNK1A1, CSNK1B, CSNK1G1, CSNK1G2, CSNK1G3, CSNK1D, CSNK1E) and/or ARK5/NUAK1 or a homologue or functional fragment thereof. In some embodiments, a subject has a cancer with at least one genetic alteration in at least one member of the casein kinase 1 family (e.g., a mutation or SNP in at least one of the following genes, CSNK1A1, CSNK1B, CSNK1D, CSNK1E, CSNK1G1, CSNK1G2, CSNK1G3). 18.3% of subjects with a genetic alteration (e.g., SNP or or genetic mutation) in a casein kinase gene can develop ovarian cancer (see FIG. 10 herein). Accordingly, in some embodiments, a subject treated with a a compound of formula (I)-(VII), such as N1-methyl 1,9-pyrazoloanthrone as disclosed herein has at least one genetic alteration in at least one member of the casein kinase 1 family (e.g., a mutation or SNP in at least one of the following genes, CSNK1A1, CSNK1B, CSNK1D, CSNK1E, CSNK1G1, CSNK1G2, CSNK1G3). In such embodiments, such a subject can have at least one of the following cancers: ovarian cancer, prostate cancer, bladder cancer, melanoma, pancreatic cancer, sarcoma, liver cancer, stomach cancer, breast cancer, uterine cancer or adenoid cancer.

In some embodiments, a subject treated with a a compound of formula (I)-(VII), such as N1-methyl 1,9-pyrazoloanthrone as disclosed herein has at least one genetic alteration (e.g., a SNP or or genetic mutation) in ARK3/NUAK1. In such embodiments, such a subject can have at least one of the following cancers: pancreatic cancer, melanoma, prostate cancer, sarcoma, stomach cancer, lung cancer, uterine cancer, colorectal cancer, colon cancer, esophageal cancer and/or bladder cancer.

In some embodiments, a biological sample is harvested from a subject with cancer to determine if the cancer expresses at least one kinase of the Casein Kinase I (CKI) family (e.g., CSNK1A1, CSNK1B, CSNK1G1, CSNK1G2, CSNK1G3, CSNK1D, CSNK1E) and/or ARK5/NUAK1, or if the subject has at least one genetic alteration (e.g. mutation or SNP) in ARK5/NUAK1 gene and/or at least one member of the casein kinase 1 family (e.g., a mutation or SNP in at least one of the following genes, CSNK1A1, CSNK1B, CSNK1D, CSNK1E, CSNK1G1, CSNK1G2, CSNK1G3). In some embodiments, the biological sample is a tissue sample, for example a cancer or tumor tissue sample or a cancer cell or tumor cell, or a biopsy tissue sample. In some embodiments, where the sample is being tested for a genetic alteration in a member of the CK1 family and/or ARK5/NUAK1, the biological sample is blood, saliva, plasma, or urine or any sample obtained from the subject comprising the subjects cells.

In some embodiments, the methods to treat a cancer as disclosed herein are useful for the treatment where the cancer comprises, for example but not limited to, an ovarian cancer cell, a vulvar epidermal carcinoma cell, a cervical carcinoma cell, an endometrial edenocarinaoma cell and/or an ovarian adenocarcinoma cell. In alternative embodiments, the methods to treat a cancer as disclosed herein are useful for the treatment of cancers such as, but not limited to, breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, gum cancer, kidney cancer, liver cancer, nasopharynx cancer, ovarian cancer, prostate cancer, skin cancer, stomach cancer, testis cancer, tongue cancer, or uterine cancer. In some embodiments, the cancer is a multi-drug resistant cancer, for example, a paclitaxel-resistant cancer. In some embodiments, the cancer comprises a cancer stem cell, such as, but not limited to an ovarian cancer stem cell.

In some embodiments, the subject with a cancer has been previously treated with chemotherapeutic agents, such as, but not limited to paclitaxel, cisplatin, doxorubicin and vermurafib.

In some embodiments in the methods to treat a cancer as disclosed herein, one can measure the expression of the nucleic acid gene product or protein or polypeptide gene product of the at least one kinase of the Casein Kinase I (CKI) family (e.g., CSNK1A1, CSNK1B, CSNK1G1, CSNK1G2, CSNK1G3, CSNK1D, CSNK1E) and/or ARK5/NUAK1MIS.

In some embodiments in the methods to treat a cancer as disclosed herein, one can administer the N1-methyl pyrazoloanthrone e.g., N1-methyl 1,9-pyrazoloanthrone, or a functional derivative or a functional analogue thereof by intravenous, intradermal, intramuscular, intra-arterial, intralesional, percutaneous, subcutaneous, or by aerosol. In some embodiments, the administering is prophylactic administration, and in alternative embodiments, the administering is therapeutic administration.

In some embodiments in the methods to treat a cancer as disclosed herein, the subject is a mammal, such as for example but not limited to, a human.

In some embodiments in the methods to treat a cancer as disclosed herein, one or more additional agents can be administered to the subject in addition to the N1-methyl pyrazoloanthrone, e.g., N1-methyl 1,9-pyrazoloanthrone or a functional derivative or a functional analogue thereof, for example, where the agents are therapeutic agents such as chemotherapeutic agents. In some embodiments, the chemotherapeutic agents include, but are not limited to, paclitaxel, cisplatin, doxorubicin, rapamycin or vermurafib, as well as MIS and recombinant forms of MIS (e.g., recombinant human MIS or rhMIS). In alternative embodiments, an additional agent can be, for example, Mullerian Inhibiting Substance (MIS) or a functional derivative or variant thereof, such as recombinant human MIS (rhMIS). In some embodiments, the additional agent is a radiotherapeutic agent.

In some embodiments in the methods to treat a cancer as disclosed herein, one can administer the pyrazoloanthrone or a functional derivative or a functional analogue thereof more than once. In some embodiments, a N1-methyl pyrazoloanthrone e.g., N1-methyl 1,9-pyrazoloanthrone, or functional derivative or functional analogue can be administered before, after or at the same time as the additional agent, and in some embodiments, an additional therapeutic agent can be administered more than once via any route commonly known by persons of ordinary skill in the art, such as intravenous, intradermal, intramuscular, intra-arterial, intralesional, percutaneous, subcutaneous, or by aerosol administration.

Another aspect of the present invention relates to a pharmaceutical composition comprising at least one N1-methyl pyrazoloanthrone e.g., N1-methyl 1,9-pyrazoloanthrone, or at least one functional derivatives thereof, which can be used alone or in combination with additional agents. In alternative embodiments, the pharmaceutical composition comprising N1-methyl pyrazoloanthrone e.g., N1-methyl 1,9-pyrazoloanthrone, or functional derivatives thereof can be used in combination with other therapeutic agents and additional therapies. In some embodiments, the additional therapies are, for example but not limited to chemotherapy, radiotherapy, thermotherapy, immunotherapy, hormone therapy, surgery and laser therapy.

In some embodiments, the present invention relates to a pharmaceutical composition comprising an inhibitor of at least one member of the CK1 family and/or ARK5/NUAK1 and a pharmaceutical acceptable carrier, for example where the inhibitor is a N1-methyl pyrazoloanthrone e.g., N1-methyl 1,9-pyrazoloanthrone, or a functional derivative or functional analogue thereof. In some embodiments, the pharmaceutical composition can further comprise one or more additional agents, such as therapeutic agents for example, chemotherapeutic agents and/or a radiotherapeutic agent. In some embodiments, a chemotherapeutic agents which can be included in the pharmaceutical composition can be, for example but are not limited to, paclitaxel, cisplatin, doxorubicin, rapamycin or vermurafib, as well as MIS and recombinant forms of MIS (e.g., recombinant human MIS or rhMIS), or analogues or functional derivatives thereof. In some embodiments, an additional agent which can be added to the pharmaceutical compositions as disclosed herein can be, for example, Mullerian Inhibiting Substance (MIS) or a functional derivative or functional variant thereof, such as recombinant human MIS (rhMIS). Other examples of therapeutic agents which can be added or administered to the subject in addition to the pharmaceutical compositions as disclosed herein can be, but are not limited to, are paclitaxel, cisplatin, doxorubicin and vermurafib, as well as MIS and recombinant forms of MIS (e.g., recombinant human MIS or rhMIS), and any other or combination of chemotherapy agents commonly known by person of ordinary skill in the art. In some embodiments, the therapeutic agent is MIS and/or recombinant or modified version of MIS, for example rhMIS, or a functional derivatives of MIS, as disclosed in International Patent Application WO92/18153, which is incorporated herein in its entirety by reference. In some embodiments, the other therapeutic agent is in interferon, for example as disclosed in U.S. Patent Application 2004/0151693, which is incorporated herein in its entirety by reference.

Accordingly, the methods of the present invention are directed to use of N1-methyl pyrazoloanthrone, e.g., N1-methyl 1,9-pyrazoloanthrone, and functional derivatives thereof with other therapeutic agents, for example chemotherapy agents, wherein the chemotherapy agents, for example paclitaxel and/or MIS can be used at a lower dose as compared to when they are used in the absence of the pyrazoloathrone or functional derivative thereof. Accordingly, where the chemotherapeutic, such as paclitaxel, cisplatin, doxorubicin and vermurafib, as well as MIS and recombinant forms of MIS (e.g., recombinant human MIS or rhMIS) are used at a lower dose, this often results in decreased side effects associated with use of such chemotherapeutics such as paclitaxel or MIS.

Another aspect of the present invention relates to methods of increasing the sensitivity of a tumor cell to chemotherapeutic agent, the method comprising administering to the cell a therapeutically effective amount of a N1-methyl pyrazoloanthrone, e.g., N1-methyl 1,9-pyrazoloanthrone, or functional derivative or functional analogue thereof. A similar aspect of the present invention relates to a method of decreasing the normal therapeutic dose of a chemotherapeutic agent for the treatment of cancer, the method comprising administering to the subject a therapeutically effective amount of a N1-methyl pyrazoloanthrone, e.g., N1-methyl 1,9-pyrazoloanthrone or functional derivative or functional analogue thereof and a chemotherapeutic agent, wherein the therapeutically effective dose of the chemotherapeutic agent (e.g., anti-cancer agent) in the presence of a N1-methyl pyrazoloanthrone, e.g., N1-methyl 1,9-pyrazoloanthrone, is lower or at a decreased dose as compared to the therapeutically effective dose of the chemotherapeutic agent when used alone or in the absence of the pyrazoloanthrone or functional derivative or functional analogue thereof. For example, in some embodiments, the effective amount of a chemotherapeutic agent to treat cancer and/or to reduce a tumor size when used in combination with a a compound of formula (I)-(VII), such as N1-methyl 1,9-pyrazoloanthrone is at least 10%, or at least 20% or at least 30% or at least 40%, or at least 50% less (e.g. a decreased dose) than the effective amount of the same chemotherapeutic agent when it is used alone and/or in the absence of N1-methyl-pyrazoloanthrone, e.g., a compound of formula (I)-(VII), such as N1-methyl 1,9-pyrazoloanthrone. Similarity, in some embodiments, the effective amount of a compound of formula (I)-(VII), such as N1-methyl 1,9-pyrazoloanthrone is at least 10%, or at least 20% or at least 30% or at least 40%, or at least 50% less than it's effective amount when used alone and/or in the absence of a chemotherapeutic agent for the treatment of cancer and/or to reduce a tumor size.

In some embodiments, the N1-methyl pyrazoloanthrone which is used to increase the sensitivity of a tumor cell, and/or decrease the dose of a chemotherapeutic agents is N1-methyl 1,9-pyrazoloanthrone (M-SB600), or derivative or analogue thereof. In some embodiments, such a chemotherapeutic agent is, for example, but not limited to, paclitaxel, cisplatin, doxorubicin, rapamycin and vermurafib, as well as MIS and recombinant forms of MIS (e.g., recombinant human MIS or rhMIS or LR-MIS or other variants) or functional derivatives thereof. In alternative embodiments, the chemotherapeutic agent is Mullerian Inhibiting Substance (MIS) or a functional derivative or variant thereof, such as, for example but not limited to, recombinant human MIS (rhMIS). In some embodiments, a N1-methyl pyrazoloanthrone e.g., N1-methyl 1,9-pyrazoloanthrone, or functional derivative or functional analogue thereof can be administered to the subject or to the tumor cell at the same time, or prior to, or following the administration of a chemotherapeutic agent or anti-cancer agent.

In some embodiments, the present invention provides methods for manufacture of a medicament for treating cancer and/or reducing the proliferation of cancer cells and/or size of a cancer, where the medicament comprises a pharmaceutical composition of N1-methyl pyrazoloanthrone, e.g., N1-methyl 1,9-pyrazoloanthrone or a functional derivatives thereof, with or without additional therapeutic agents, as discussed above. In some embodiments, the cancer cell expresses at least one member of the CK1 family and/or ARK5/NUAK1. Another embodiment relates to a method for manufacture of a medicament for inhibiting at least one member of the CK1 family and/or ARK5/NUAK1, where the medicament comprises a pharmaceutical composition comprising a N1-methyl pyrazoloanthrone e.g., N1-methyl 1,9-pyrazoloanthrone or functional derivative thereof, in the presence or absence of an additional chemotherapeutic agent as disclosed herein.

In some embodiments, the N1-methyl pyrazoloanthrone, e.g., N1-methyl 1,9-pyrazoloanthrone or functional derivatives thereof as disclosed herein can be formulated as a pharmaceutical composition which contains an effective dosage amount of one or more pyrazoloathrone and functional derivatives thereof in combination with one (or more) pharmaceutically acceptable carrier(s). Conditions that may be treated with the N1-methyl pyrazoloanthrone, e.g., N1-methyl 1,9-pyrazoloanthrone and functional derivatives thereof of this invention, or a pharmaceutical composition containing the same and addition of other therapeutic agents, include any condition which may benefit from administration of an inhibitor of at least one member of the CK1 family and/or ARK5/NUAK1, and are particularly useful for the prevention and/or treatment of various diseases, for example proliferative diseases. In some embodiments, the proliferative disease is cancer, and can be, for example, but are not limited to cancers or cancer stem cells expressing at least one member of the CK1 family, and/or ARK5/NUAK1 or MIS receptors, for example cancers or cancer stem cells expressing MISRII.

Examples of cancers which can be treated with the N1-methyl pyrazoloanthrone, e.g., N1-methyl 1,9-pyrazoloanthrone and functional derivatives as disclosed herein are, for example, but not limited to, any cancer comprising a cancer stem cell, and/or ovarian cancer, vulvar epidermal carcinoma, cervical carcinoma, endometrial edenocarinaoma and ovarian adenocarcinoma. In some embodiments, the cancer is, for example but not limited to, breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, genital-urinary cancer, stomach cancer, lymphomas, melanoma, glioma, bladder cancer, pancreatic cancer, gum cancer, kidney cancer, retinal cancer, liver cancer, nasopharynx cancer, ovarian cancer, oral cancers, bladder cancer, hematological neoplasms, follicular lymphoma, cervical cancer, multiple myeloma, osteosarcomas, thyroid cancer, prostate cancer, colon cancer, prostate cancer, skin cancer, stomach cancer, testis cancer, tongue cancer, or uterine cancer.

Another aspect of the present invention relates to the use of a N1-methyl pyrazoloanthrone, e.g., N1-methyl 1,9-pyrazoloanthrone or derivative or analogue thereof for the manufacture of a medicament for treating cancer, wherein the cancer expresses at least one member of the CK1 family and/or ARK5/NUAK1. In some embodiments, the cancer cell also expresses a MIS type II receptor (MISRII) or a homologue or functional fragment thereof.

Another aspect of the present invention relates to an article of manufacture comprising packaging material and a pharmaceutical composition comprising N1-methyl pyrazoloanthrone, e.g., N1-methyl 1,9-pyrazoloanthrone or functional derivatives thereof as disclosed herein, wherein the packaging material comprises a label which indicates the pharmaceutical composition may be administered, for a sufficient term at an effective dose, for treating or reducing the risk of cancer which expresses a Mullerian Inhibiting Substance (MIS) receptor.

Another aspect of the present invention relates to a method of treating a subject affected with cancer, the method comprising assessing the expression and/or activity of Mullerian Inhibiting Substance Receptor II (MISRII) in a biological sample obtained from the subject, wherein a clinician reviews the results and if the results indicate the presence of expression and/or activity of MISRII, the clinician directs the subject to be treated with pharmaceutical composition as disclosed herein. In some embodiments, a biological sample obtained from the subject is a tissue sample, for example a cancer or tumor tissue sample or a cancer cell or tumor cell, such as a biopsy tissue sample. In some embodiments, the cancer tissue sample comprises an ovarian cancer cell, a vulvar epidermal carcinoma cell, a cervical carcinoma cell, an endometrial edenocarinaoma cell and/or an ovarian adenocarcinoma cell. In some embodiments, the cancer tissue sample is from a cancer, such as, but not limited to, breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, gum cancer, kidney cancer, liver cancer, nasopharynx cancer, ovarian cancer, prostate cancer, skin cancer, stomach cancer, testis cancer, tongue cancer, or uterine cancer.

Another aspect of the present invention relates to the use of a N-methyl pyrazoloanthrone or derivative or analogue thereof, e.g., N1-methyl-1,9-pyrazoloathrone is to decrease the plasma serum levels of one or more androgens, such as but not limited to, testosterone, in a subject in need thereof. In some embodiments, a N-methyl pyrazoloanthrone or derivative or analogue thereof is N1-methyl-1,9-pyrazoloathrone or a derivative or analogue thereof. Other indications which can be treated with a N-methyl pyrazoloanthrone or derivative or analogue thereof, e.g., N1-methyl-1,9-pyrazoloathrone are conditions where excess androgen is present, for example, but not limited to, rheumatoid arthritis, proliferative diseases such as cancer, treatment of prostatic cancer, polycysic ovarian disease, benign prostatic hypertrophy and precocious puberty and other hyperandrogen disorders such as testitoxicosis. In some embodiments, a N-methyl pyrazoloanthrone or derivative or analogue thereof, e.g., N1-methyl-1,9-pyrazoloathrone can be used as a method of contraception in a female subject and/or to protect ovarian reserves in a female subject.

These and other aspects of this invention will be apparent upon reference to the following detailed description. To that end, certain patent and other documents are cited herein to more specifically set forth various aspects of this invention. Each of these documents are hereby incorporated by reference in their entirety.

### BRIEF DESCRIPTION OF FIGURES

FIG. 1A-1B shows the chemical structures of 1,9-pyrazoloathrone and N1-methyl-1,9-pyrazoloathrone. FIG. 1A shows the chemical structure of 1,9-pyrazoloathrone (also referred to herein as “SP600125” or “SP600”). FIG. 1B shows the chemical structure of N1-methyl-1,9-pyrazoloathrone (also referred to herein as “M-SP600”).

FIG. 2A-2B shows that M-SP600 is not an inhibitor of JNK or a MIS mimic FIG. 2A shows the treatment of PtD cells (a primary ovarian cancer cell ascite cell line) treated for 30 minutes with 25 μM of SP600 or JNKi VIII but not M-SP600 or controls result in inhibition of c-jun phosphorylation. FIG. 2B shows ex-vivo treatment of urogenital ridges dissected from female fetal rats E14.5 with SP600 (25 μM) but not M-SP600 (25 μM) results in regression of the Mullerian duct.

FIG. 3 shows the dose response curve of SP600 and M-SP600 in a variety of cell lines. Primary ovarian cancer ascite cell lines (PKD1, ptD, PKD2, ptG and ptH) were treated with SP600 (blue), M-SP600 (red) or DMSO control (black) in a variety of doses for 72 hours in a 96-well plate and then cell death determined by MTT assay.

FIGS. 4A-4C shows M-SP600 is synergistic with other chemotherapeutic treatments and targeted anti-cancer therapies. FIG. 4A shows a combination of treatment of M-SP600 with vemurafinib, a specific B-RAF inhibitor targeted at tumors bearing V600E mutations in ptD cells by MTT. FIG. 4B shows ptD cells bearing the V600E B-RAF mutation (SEQ ID NOS 7 and 8, respectively, in order of appearance). FIG. 4C show that the combination of M-SP600 and Vemurafinib is synergistic at low and high doses.

FIGS. 5A-5C shows cell cycle changes induced by SP600 and M-SP600 treatment. FIG. 5A shows treatment of asynchronous ptD primary ovarian cancer cells with 25 μM of SP600, 25 μM of M-SP600 or DMSO control for 24 hours and cell cycle distribution was analyzed with P1 staining and flow cytometry. FIG. 5B shows ptD cells which were serum starved for 24 h and synchronized by adding 10% serum with DMSO, 25 μM SP600, 25 μM MSP600 and cell cycle distribution was analyzed with PI staining by flow cytometry at 0, 2, 4 12 and 24 h. FIG. 5C shows OVCAR5 cells which were serum starved for 24 h and synchronized by adding 10% serum with DMSO, 25 uM SP600, 25 μM MSP600 and cell cycle distribution was analyzed with PI staining by flow cytometry at 0, 2, 4 12 and 24 h.

FIG. 6 shows expression of the CDKi P21 in ptD cells by quantitative PCR (qPCR). ptD cells were treated in 2 well plates with either 25 μM of SP600, 25 μM of M-SP600 or DMSO control for 0, 2, 4, 8, 12 and 48 hours. 2̂-(dCt) values are shown.

FIG. 7 shows the spectrum of kinase inhibition of SP600 and M-SP600. All compounds were tested at a concentration of 0.5 μM in the presence of 10 μM ATP with 300 different kinases and their substrates. Kinases that had over 50% inhibition are displayed in this Figure.

FIG. 8A-8B shows patient-derived xenograft model of ovarian cancer in nude mice treated with DMSO control, SP600 or M-SP600. FIG. 8A shows mice implanted with 1×10⁶ ptD cells which were grown for one week and then treated with 30 mg/kg/day of SP600, M-SP600 or control (DMSO) for 3 consecutive days (days 7, 8 and 9). FIG. 8B shows mice implanted with 1×10⁶ OVCAR5 cells and 72 hours later treated with 600 mg/kg/day of SP600 or M-SP600 (or control) for 3 consecutive weeks, for 5 days per week. Tumor volume was measured by calipers, and mice were sacrificed on day 18. N1-Me-pyrazoloanthrone (“MSP600”) tumor growth is significantly lower than control by two-way ANOVA (p<0.05). FIG. 8C shows Kaplan-Meyner survival plot of mice implanted shows mice implanted with 1×10⁶ ptD cells which were grown for one week and then treated with 30 mg/kg/day of SP600, M-SP600 or control (DMSO) for 3 consecutive days (days 7, 8 and 9).

FIGS. 9A-9B show a dose response to combination therapy of M-SP600 and Doxorubicin (DOX). FIG. 9A shows a MTT dose response over a72 h incubation with increasing concentration of doxorubicin (DOX) (from 0-360 μM), M-SP600 (from 0-60 μM), or a combination of both DOX and M-SP600 in OVCAR5 cells, showing virtually complete cell death (e.g., 95% cell death) with the combination of DOX and M-SP600 at 180 μM and 30 μM doses respectively, where the same level of cell death is not achieved with use of DOX alone until a dose of 300 μM or with the use of M-SP600 alone until a dose of 50 μM. FIG. 9B shows a MTT dose response over a 72 h incubation with increasing concentration of doxorubicin (DOX), M-SP600 or a combination of both DOX and M-SP600 in ptD primary ovarian cancer cells, showing virtually complete cell death (e.g., 95% cell death) with the combination of DOX and M-SP600 at 140 μM and 25 μM doses respectively, where the same level of cell death is not achieved with use of DOX alone until a dose of greater than 300 μM or with the use of M-SP600 alone until a dose of 50 μM.

FIG. 10 is a schematic diagram showing cancers with genetic alterations to the casein kinase family members (CSNK1A1, CSNK1B, CSNK1D, CSNK1E, CSNK1G1, CSNK1G2, CSNK1 G3). Data adapted from the TCGA study showing frequent alteration of casein kinase genes, particularly in ovarian cancer.

### DETAILED DESCRIPTION

As discussed herein, the present invention provides a method for treating a variety of conditions by administering an effective amount of a N-methyl pyrazoloathrone, such as N1-methyl pyrazoloathrone and functional derivatives thereof of the invention to a subject in need thereof. Conditions that may be treated by the compounds of this invention, or a pharmaceutical composition containing the same, include any condition which is treated or has a reduction in a symptoms by administration of MIS or activation of MIS signaling or activation of MISRII, and thereby benefit from administration of a N-methyl pyrazoloathrone, such as N1-methyl pyrazoloathrone and functional derivatives thereof. Representative conditions in this regard include, for example, but not limited to, cancers that express MIS receptors, for example cancer that express MISRII, for example, but not limited to ovarian, cervical and endometrial cancer.

Other conditions which can be treated with a N-methyl pyrazoloanthrone or derivative or analogue thereof, e.g., N1-methyl-1,9-pyrazoloathrone or benefit from MIS or activation of MIS signaling includes cancers which expresses at least one member of the CK1 family and/or ARK5/NUAK1.

Other indications which can be treated with a N-methyl pyrazoloanthrone or derivative or analogue thereof, e.g., N1-methyl-1,9-pyrazoloathrone are conditions where excess androgen is present, for example, but not limited to, rheumatoid arthritis, proliferative diseases such as cancer, treatment of prostatic cancer, polycysic ovarian disease, benign prostatic hypertrophy and precocious puberty and other hyperandrogen disorders such as testitoxicosis. In some embodiments, a N-methyl pyrazoloanthrone or derivative or analogue thereof, e.g., N1-methyl-1,9-pyrazoloathrone can be used as a method of contraception in a female subject and/or to protect ovarian reserves in a female subject. In all aspects of the present invention, a N-methyl pyrazoloanthrone or derivative or analogue thereof, e.g., N1-methyl-1,9-pyrazoloathrone can be administered alone, or in combination with additional agents, such as chemotherapeutic agents, such as hMIS or a variant thereof, such as a LR-MIS variant of SEQ ID NO: 1. In some embodiments, a N-methyl pyrazoloanthrone or derivative or analogue thereof, e.g., N1-methyl-1,9-pyrazoloathrone is administered in combination with hMIS or MIS variant, the hMIS or LR-MIS variant can be expressed by a viral vector, e.g., adenoviral vector, a poxvirus vector, or lentiviral vector, or AAV vector such as AAV9.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “CK1” refers to the Casein kinase 1 family of protein kinases which are serine/threonine-selective enzymes that function as regulators of signal transduction pathways in most eukaryotic cell types. Without wishing to be bound by theory, CK1 isoforms are involved in Wnt signaling, circadian rhythms, nucleo-cytoplasmic shuttling of transcription factors, DNA repair, and DNA transcription. Mammals have seven family members (sometimes referred to as isoforms, but encoded by distinct genes): alpha (CK1α or CK1α1 or CSNK1A1), beta (CK1β or CK1b or CSNK1B), gamma 1 (CKγ1 or CK1gamma1 or CSNK1G1), gamma 2 (CK1γ2 or CK1gamma2 or CSNK1G2), gamma 3 (CK1γ3 or CK1gamma3 or CSNK1G3), delta (CK1δ or CK1d or CSNK1D), and epsilon (CK1E or CK1e or CSNK1E). Isoforms range from 22 to 55 kDa and have been identified in the membranes, nucleus, and cytoplasm of eukaryotes and additionally in the mitotic spindle in mammalian cells. For reference purposes only, CK1α is also known as CSHK1A1 and includes isforms with differing protein lengths (CK1α1 which is 337 (UniProt ID P48729) or CK1a2 which is 365 amino acids (UniProt ID P48729-2), and the human CK1α protein corresponds to the amino acid sequence of NP_001883.4, which is encoded by the nucleic acid corresponding to RefSeq:NM_001892, which are incorporated herein by reference. CK1β is also known as CSNK1B, and the human CK1β protein corresponds to the amino acid sequence of NP_001091628.1, which is encoded by the nucleic acid corresponding to NM_001098159.1, which are incorporated herein by reference. CK1γ1 is also known as CSNK1 G1 and includes isforms with differing protein lengths (CK1γ1.v1 which is 393 (UniProt ID Q9HCP0) or CK1γ1.v2 which is 422 amino acids (UniProt ID Q9HCP0-2), and the human CK1γ1 protein corresponds to the amino acid sequence of NP_071331.2, which is encoded by the nucleic acid corresponding to RefSeq:NM_022048, which are incorporated herein by reference. CK1γ2 is also known as CSNK1G2 and includes isforms with differing protein lengths (CK1γ2.v1 or CK1γ2.v2 (UniProt ID 78368), and the human CK1γ2 protein corresponds to the amino acid sequence of NP_001310.3, which is encoded by the nucleic acid corresponding to RefSeq:NM_001319, which are incorporated herein by reference. CK1γ3 is also known as CSNK1 G3, and includes at least 6 different isforms with differing protein lengths from 311-445 amino acids (CK1γ3.v1 (UniProt ID Q9Y6M4), CK1γ3.v2 (UniProt ID Q9Y6M4-2), CK1γ3.v3 (UniProt ID Q9Y6M4-3), CK1γ3.v4 (UniProt ID Q9Y6M4-4), CK1γ3.v5 (UniProt ID Q9Y6M4-5), CK1γ3.v6 (UniProt ID Q9Y6M4-6)), and the human CK1γ3 protein corresponds to the amino acid sequence of NP_004375.2, which is encoded by the nucleic acid corresponding to RefSeq:NM_004384, which are incorporated herein by reference. CK1δ is also known as CSNK1D, CKID, CKIdelta, or HCKID, and includes isforms with differing protein lengths (CK1δ1 which is 409 (UniProt ID P48703) or CK1δ2 which is 415 amino acids (UniProt ID P48703-2), and the human CK1δ protein corresponds to the amino acid sequence of NP_620693.1, which is encoded by the nucleic acid corresponding to RefSeq: NM_139062, which are incorporated herein by reference. CK1E is also known as CSNK1E, CKIE, CK1epsilon, HCKIE, and the human CK1E protein of 416 amino acids corresponds to the amino acid sequence of NP_001885.1, which is encoded by the nucleic acid corresponding to RefSeq: NM_001894, which are incorporated herein by reference.

The term “ARK5/NUAK1” refers to the NUAK family SNF1-like kinase 1 also known as AMPK-related protein kinase 5 (ARK5), and is an enzyme that in humans is encoded by the NUAK1 gene. ARK5 is also known as NUAK1, and the human NUAK1 protein corresponds to the amino acid sequence of NP_055655.1, which is encoded by the nucleic acid corresponding to RefSeq:NM_014840, which are incorporated herein by reference.

The term “Mullerian Inhibiting Substance” and “MIS” are used interchangeably herein and is also known as anti-Müllerian hormone or AMH, refer to compounds and materials which are structurally similar to MIS. Examples of such intended substances are for example, salts, functional derivatives and aglycone forms of MIS. Additionally, the present invention is intended to include mutant forms of MIS which have substantially the same biological activity as MIS. Examples of such mutant MIS molecules carrying a deletion, insertion, or alteration in amino acid sequence. MIS can be obtained from any mammalian source or from non-mammalian sources through the use of recombinant DNA technology, or from chemical synthesis of the MIS protein. For reference purposes only, the human MIS nucleic acid corresponds to ReSeq No: NM_000479 (SEQ ID NO:2) and GenBank No: KO3474 (SEQ ID NO:3), which are incorporated herein by reference.

The term “Mullerian Inhibiting Substance type II receptor” or “MISRII” are used interchangeably herein refer to the type II receptor for MIS. The term MISRII is intended to encompass all MIS receptors substantially homologous to MISRII and functional derivatives of MISRII. MISRII is also known by the alias as AMHR2, and for reference purposes, the nucleic acid sequence of human MISRII corresponds to NM_020547 (SEQ ID NO:4) and GenBank No: AF172932 (SEQ ID NO:5) which are incorporated herein by reference

The term “functional derivative” and “mimetic” are used interchangeably herein, and refers to compounds which possess a biological activity (in particular functional biological activity) that is substantially similar to the biological activity of the entity or molecule for which it's a functional derivative of. The term functional derivative is intended to include the fragments, variants, analogues or chemical derivatives of a molecule. In certain embodiments, functional derivatives and functional analogues of N1-methyl pyrazoloanthrone, e.g., N1-methyl 1,9-pyrazoloanthrone as disclosed herein can be assessed for their biological activity using an assay to assess the ability of the derivatives and analogues inhibit at least one member of the CK1 family and/or ARK5/NUAK1 to at least 80%, or at least 90% as effective, or more than 90%, e.g., about 1.2-fold, or 1.5-fold, or 2-fold or more than 2-fold more effective at inhibiting at least one member of the CK1 family and/or ARK5/NUAK1 as N1-methyl 1,9-pyrazoloanthrone is considered as functional derivative or functional analogue of N1-methyl 1,9-pyrazoloanthrone.

The term “analog” as used herein refers to an agent that retains the same, or a substantially similar biological function (i.e., inhibition of at least one member of the CK1 family and/or ARK5/NUAK1) and/or structure as the molecule or chemical or polypeptide it is an analogue of Examples of analogs include peptidomimetics (a peptide analog), peptide nucleic acids (a nucleic acid analog), small and large organic or inorganic compounds, as well as derivatives and variants of a polypeptide or nucleic acid herein.

The term “substantially similar”, when used to define the biological activity of a derivative or analogue of N1-methyl 1,9-pyrazoloanthrone as compared to the biological activity of N1-methyl 1,9-pyrazoloanthrone to which it is a derivative or analogue of, means that a particular derivative or analogue differs from the initial N1-methyl 1,9-pyrazoloanthrone in chemical structure, by one or more groups or elements, including substitutions, deletions, or additions of groups of elements, the net effect of which is to retain at least some of the biological activity found in the initial N1-methyl 1,9-pyrazoloanthrone with respect to the biological activity of N1-methyl 1,9-pyrazoloanthrone with respect to activation of the inhibition of at least one member of the CK1 family and/or ARK5/NUAK1. Such biological activity can be assessed by one of ordinary skill in the art using the assay as disclosed herein. As such, derivative or analogue of N1-methyl 1,9-pyrazoloanthrone having lesser degrees of structural similarity but a substantially similar or comparable biological activity of the original N1-methyl 1,9-pyrazoloanthrone from which is based with respect to inhibition of at least one member of the CK1 family and/or ARK5/NUAK1 are considered to be equivalents. Substantially similar derivatives or analogues of N1-methyl 1,9-pyrazoloanthrone will typically have at least about 60%, or at least about 70% or at least about 80% or at least about 90% or at least about 95%, or at least about 100% the biological activity of inhibition of at least one member of the CK1 family and/or ARK5/NUAK1 as compared to the N1-methyl 1,9-pyrazoloanthrone it is a derivative or analogue of, or at least at least 2-fold, or at least about 3-fold, or at least about a 4-fold, or at least about a 5-fold or at least about a 10-fold, or any increase between 2-fold and 10-fold or greater the biological activity of inhibition of at least one member of the CK1 family and/or ARK5/NUAK1 as compared to the N1-methyl 1,9-pyrazoloanthrone are to be considered a functional derivative or a functional analogue of the N1-methyl 1,9-pyrazoloanthrone they are based on, as can be assayed using the methods as disclosed herein.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “subject” and “individual” are used interchangeably herein, and refer to an animal, for example a human, to whom treatment for cancer or a proliferative disorder, including therapeutic treatment or prophylactic treatment, with a pharmaceutical composition comprising a compound of (I)-(IV), such as, e.g., a N1-methyl pyrazoloanthrone, e.g., N1-methyl 1,9-pyrazoloanthrone as disclosed herein can be administered. The term “subject” as used herein includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term “non-human animals” and “non-human mammals” are used interchangeably herein and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. In one embodiment, the subject is human. In another embodiment, the subject is an experimental animal or animal substitute as a disease model, including transgenic non-human animal species.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, inderdisposition, affection.

The term “cancer” and “malignancy” are used interchangeably herein, refers to diseases that are characterized by uncontrolled, abnormal growth of cells. In some embodiments, the term cancer encompasses cancer cells which have spread locally or through the bloodstream and lymphatic system to other parts of the body, referred to herein as “metastatic cancer”. The term is also intended to include any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakia's which often precede a breakout of cancer.

As used herein, the term “tumor” refers to a mass of transformed cells that are characterized, at least in part, by containing angiogenic vasculature. The transformed cells are characterized by neoplastic uncontrolled cell multiplication which is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e. non-metastatic tumor). Tumors are hallmarks of cancer, a neoplastic disease the natural course of which is fatal. Cancer cells exhibit the properties of invasion and metastasis and are highly anaplastic.

As used herein, the terms “metastases” or “metastatic tumor” “metastatic cancer” are used interchangeably herein and refer to a secondary tumor that grows separately elsewhere in the body from the primary tumor and has arisen from detached cancer cells from the primary tumor which have been transported to a separate location, and where the primary tumor is a solid tumor. The primary tumor, as used herein, refers to a tumor that originated in the location or organ in which it is present and did not metastasize to that location from another location. As used herein, a “malignant tumor” or “metastatic cancer” is one having the properties of invasion and metastasis and showing a high degree of anaplasia. Anaplasia is the reversion of cells to an immature or a less differentiated form, and it occurs in most malignant tumors.

The term “therapy resistant cancer” as used herein refers to a cancer present in a subject which is resistant to, or refractory to at least two different anti-cancer agents such as chemotherapy agents, which means, typically a subject has been treated with at least two different anti-cancer agents that did not provide effective treatment as that term is defined herein.

The term ‘sensitize’ or ‘sensitizes’ used interchangeably herein, refers to making the cell sensitive, or susceptible to other secondary agents, for example other pro-drugs or other environmental effects such as radiation etc. For example, as disclosed herein, a N1-methyl-pyrazoloanthrone, e.g., a compound of formula (I)-(VII), such as N1-methyl 1,9-pyrazoloanthrone as disclosed herein can sensitize a cell, e.g., a cancer cell or a cancer stem cell to increased killing of a chemotherapeutic agent, such as, but not limited to, paclitaxel, cisplatin, doxorubicin, vermurafib. Accordingly, the compositions as disclosed herein can be used to reduce the normal dose of a chemotherapeutic agent for the treatment of cancer in a subject.

The cells used in the invention can also be cultured cells, e.g. in vitro or ex vivo. For example, cells cultured in vitro in a culture medium. Alternatively, for ex vivo cultured cells, cells can be obtained from a subject, where the subject is healthy and/or affected with a disease. Cells can be obtained, as a non-limiting example, by biopsy or other surgical means know to those skilled in the art. Cells used in the invention can be present in a subject, e.g. in vivo. For the invention on use on in vivo cells, the cell is preferably found in a subject and display characteristics of the disease, disorder, or malignancy pathology

As used herein, the terms “treat” or “treatment” or “treating” refers to therapeutic treatment, wherein the object is to prevent or slow the development of the disease, such as slow down the development of a tumor, the spread of cancer, or reducing at least one effect or symptom of a condition, disease or disorder associated with inappropriate proliferation or a cell mass, for example cancer. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with cancer, as well as those likely to develop secondary tumors due to metastasis.

When used in reference to the treatment of cancer, the term “treating” is used to refer to the reduction of a symptom and/or a biochemical marker of cancer, for example a reduction in at least one biochemical marker of cancer by at least about 10% would be considered an effective treatment. Exemplary examples of such biochemical markers of cancer include, for example, but are not limited to, CD44, telomerase, TGF-α, TGF-β, erbB-2, erbB-3, MUC1, MUC2, CK20, PSA, CA125 and FOBT. A reduction in the rate of proliferation of the cancer cells by at least about 10% would also be considered effective treatment by the methods as disclosed herein. As alternative examples, a reduction in a symptom of cancer, for example, a slowing of the rate of growth of the cancer by at least about 10% or a cessation of the increase in tumor size, or a reduction in the size of a tumor by at least about 10% or a reduction in the tumor spread (i.e. tumor metastasis) by at least about 10% would also be considered as affective treatments by the methods as disclosed herein. In some embodiments, it is preferred, but not required that the therapeutic agent actually kill the tumor.

The term “prophylactic treatment” refers to the prevention of the development of cancer in a subject when the subject is at a high risk of developing cancer, such as, for example, a predisposition to cancer where the subject has a genetic mutation or polymorphism known to increase occurrence of a cancer, or a family history of cancer. In some embodiments, prophylactic treatment is used in a subject who has been successfully therapeutically treated for cancer and where the cancer has been eliminated or the subject has gone into remission, and is administered prophylactic treatment with a compound of formula (I)-(VII) as disclosed herein, such as N1-methyl 1,9-pyrazoloanthrone to prevent a cancer reoccurring and/or cancer relapse.

The term “effective amount” as used herein refers to the amount of a compound of formula (I)-(VII), such as N1-methyl 1,9-pyrazoloanthrone as disclosed herein, or a chemotherapeutic agent, to alleviate at least one or more symptom of the disease or disorder, and relates to a sufficient amount of pharmacological composition to provide the desired effect. The phrase “therapeutically effective amount” as used herein, e.g., a pharmaceutical composition comprising at least one pyrazoloanthrone as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylacticly significant reduction in a symptom or clinical marker associated with a cancer or a cancer-mediated condition.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, however, that the total daily usage of the compositions and formulations as disclosed herein will be decided by the attending physician within the scope of sound medical judgment. The exact amount required will vary depending on factors such as the type of disease being treated.

With reference to the treatment of a subject with a cancer with a pharmaceutical composition comprising at least one pyrazoloanthrones as disclosed herein, the term “therapeutically effective amount” refers to the amount that is safe and sufficient to prevent or delay the development and further growth of a tumor or the spread of metastases in cancer patients. The amount can thus cure or cause the cancer to go into remission, slow the course of cancer progression, slow or inhibit tumor growth, slow or inhibit tumor metastasis, slow or inhibit the establishment of secondary tumors at metastatic sites, or inhibit the formation of new tumor metastases. The effective amount for the treatment of cancer depends on the tumor to be treated, the severity of the tumor, the drug resistance level of the tumor, the species being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of cancer and tumor, for example treatment of a rodent with a cancer, and any treatment or administration of the compositions or formulations that leads to a decrease of at least one symptom of the cancer, for example a reduction in the size of the tumor or a slowing or cessation of the rate of growth of the tumor indicates effective treatment. In embodiments where the compositions are used for the treatment of cancer, the efficacy of the composition can be judged using an experimental animal model of cancer, e.g., wild-type mice or rats, or preferably, transplantation of tumor cells. When using an experimental animal model, efficacy of treatment is evidenced when a reduction in a symptom of the cancer, for example a reduction in the size of the tumor or a slowing or cessation of the rate of growth of the tumor occurs earlier in treated, versus untreated animals. By “earlier” is meant that a decrease, for example in the size of the tumor occurs at least 5% earlier, but preferably more, e.g., one day earlier, two days earlier, 3 days earlier, or more.

The term “synergy” or “synergistic” as used herein refers to the interaction of two or more agents so that their combined effect is greater than each of their individual effects at the same dose alone. For example, administration of a chemotherapeutic agent (e.g., vermurafib or paclitaxel, or cisplatin, MIS or a recombinant MIS protein) with a N1-methyl-pyrazoloanthrone, e.g., a compound of formula (I)-(VII), such as N1-methyl 1,9-pyrazoloanthrone as disclosed herein can function synergistically in the reduction of cancer, such that the effective amount (e.g., dose) of the chemotherapeutic agent and/or the effective amount (e.g., dose) of the N1-methyl 1,9-pyrazoloanthrone when they are used in combination for the treatment of a cancer is less than the effective amount (e.g., dose) of each agent for the treatment of cancer when they are used alone. Similarly, an inhibitor of at least one kinase of the CK1 family (e.g., CSNK1A1, CSNK1B, CSNK1G1, CSNK1G2, CSNK1G3, CSNK1D, CSNK1E) and/or ARK5/NUAK1 can function synergistically with a N1-methyl-pyrazoloanthrone, e.g., a compound of formula (I)-(VII), such as N1-methyl 1,9-pyrazoloanthrone as disclosed herein to treat cancer and/or to reduce a tumor size. By way of example only, in some embodiments, the effective amount (e.g., dose) of a chemotherapeutic agent (e.g., anti-cancer agent) to treat cancer and/or to reduce a tumor size when used in combination with a N1-methyl-pyrazoloanthrone, e.g., a compound of formula (I)-(VII), such as N1-methyl 1,9-pyrazoloanthrone is at least 10%, or at least 20% or at least 30% or at least 40%, or at least 50% less (e.g. a decreased dose) than the effective amount of the same chemotherapeutic agent when it is used alone and/or in the absence of N1-methyl-pyrazoloanthrone, e.g., a compound of formula (I)-(VII), such as N1-methyl 1,9-pyrazoloanthrone. Similarly, in some embodiments, the effective amount (e.g., dose) of N1-methyl-pyrazoloanthrone, e.g., a compound of formula (I)-(VII), such as N1-methyl 1,9-pyrazoloanthrone is at least 10%, or at least 20% or at least 30% or at least 40%, or at least 50% less than it's effective amount (e.g., dose) when used alone and/or in the absence of a chemotherapeutic agent for the treatment of cancer and/or to reduce a tumor size.

As used herein, the terms “administering,” and “introducing” are used interchangeably herein and refer to the placement of the pharmaceutical compositions of the present invention comprising a N1-methyl-pyrazoloanthrone, e.g., a compound of formula (I)-(VII), such as N1-methyl 1,9-pyrazoloanthrone as disclosed herein into a subject by a method or route which results in at least partial localization of the pyrazoloanthrones at a desired site. The compounds of the present invention can be administered by any appropriate route which results in an effective treatment in the subject.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. The phrases “systemic administration,” “administered systemically”, “peripheral administration” and “administered peripherally” as used herein mean the administration of the pharmaceutical compositions of the present invention comprising pyrazoloanthrones and optionally other agents or material other than directly into the central nervous system, such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in maintaining the activity of or carrying or transporting the subject agents from one organ, or portion of the body, to another organ, or portion of the body. In addition to being “pharmaceutically acceptable” as that term is defined herein, each carrier must also be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

t should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Compositions of Pyrazoloathrone and Derivatives

As mentioned above, the present invention is directed to compounds which are as disclosed herein which have activity as inhibitor of at least one kinase of the Casein Kinase I family (e.g., CSNK1A1, CSNK1B, CSNK1G1, CSNK1G2, CSNK1G3, CSNK1D, CSNK1E) and/or ARK5/NUAK1, as well as to compositions and methods relating to the same. In some embodiments, the present invention relates to compounds which are N1-methyl-pyrazoloanthrones, e.g., a compound of formula (I)-(VII), such as N1-methyl 1,9-pyrazoloanthrone.

In some embodiments, the compounds of this invention have the following structure (I):

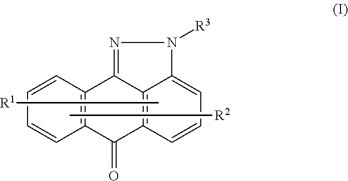

### Wherein:

R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and pharmaceutically acceptable salts thereof.

In some embodiments, R¹ and R² are both absent, i.e., compounds are of structure (II):

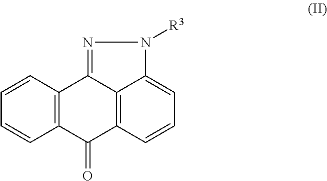

In some embodiment, only one of R¹ and R² is present, i.e., compounds are of structure (III) or (IV):

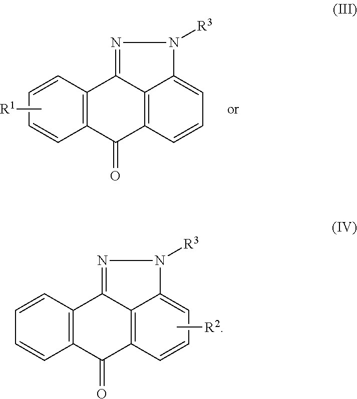

In some other embodiments, R¹ and R² are both present and are attached to the same ring, i.e., compounds of structure (V) or (VI):

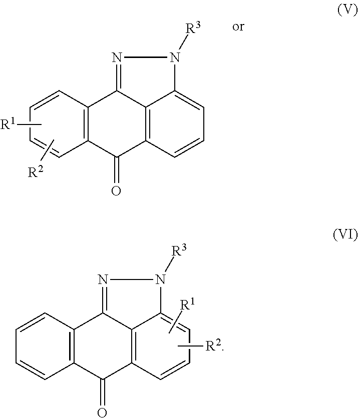

In some embodiments, R¹ and R² are both present and are attached to the different rings, i.e., compounds of structure (VII):

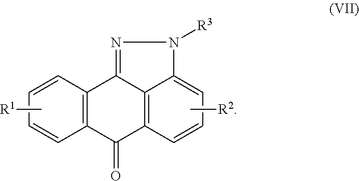

In embodiments of the compounds disclosed herein, R³ can be a C₁-C₆ alkyl. Exemplary C₁-C₆ alkyl include, but are not limited to, methyl, ethyl, n-propyl, isopropyl, 1-methyl-propyl, n-butyl, isobutyl, t-butyl, 1-methyl-butyl, pentyl, hexyl, propylenyl, 1-butenyl, propynyl, and the like. In one embodiment, R³ is methyl.

In some embodiments of the compounds disclosed herein, R³ can be an alkyl wherein backbone of the alkyl is interspersed with one or more hetero groups or atoms selected from 0, NH, S, SS, SO, SO₂, and any combinations thereof.

In some embodiments of the compounds disclosed herein, R³ can be a substituted alkyl.

In some embodiments, the compound of formula (I) is a N1-methyl-pyrazolothrone which is N1-methyl-1,9-pyrazolothrone, referred herein to as “M-SP600” or “M-SP600125” having the following structure.

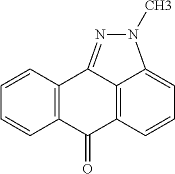 [SMILES: Cn1nc2c3c(cccc31)C(=O)c1ccccc1-2]

The compounds disclosed herein can generally be made by organic synthesis techniques known to those skilled in the art, as well as by the methods disclosed in U.S. Pat. No. 7,119,114, which is incorporated herein in its entirety by reference.

As used herein, the terms used above having following meaning.

As used herein, the term “aliphatic” means a moiety characterized by a straight or branched chain arrangement of constituent carbon atoms and can be saturated or partially unsaturated with one or more (e.g., one, two, three, four, five or more) double or triple bonds.

As used herein, the term “alicyclic” means a moiety comprising a nonaromatic ring structure. Alicyclic moieties can be saturated or partially unsaturated with one or more double or triple bonds. Alicyclic moieties can also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃-C₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2, 3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like.

Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}, —N⁺(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S— alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino”—NH-(alkyl)-(cycloalkyl), such as —NHCH₂-cyclohexyl, and the like.

“Trifluoromethyl” means —CF₃.

“Sulfonyl” means —SO₃H.

“Carboxyl” means —COOH.

“Alkoxy” means —O-(alkyl), such as methoxy, ethoxy, n-propyloxy, iso-propyloxy, n-butyloxy, iso-butyloxy, and the like.

“Alkoxyalkoxy” means —O-(alkyl)-O-(alkyl), such as —OCH₂CH₂OCH₃, and the like.

“Alkoxycarbonyl” means —C(|O)O-(alkyl), such as —C(═O)OCH₃, —C(═O)OCH₂CH₃, and the like.

“Alkoxyalkyl” means -(alkyl)-O-(alkyl), such as —CH₂OCH₃, —CH₂OCH₂CH₃, and the like.

“Arylalkyl” means -(alkyl)-(aryl), such as benzyl (i.e., —CH₂phenyl), —CH₂-pyrindinyl, and the like.

“Arylalkyloxy” means —O-(alkyl)-(aryl), such as —O-benzyl, —O—CH₂-pyridinyl, and the like.

“Cycloalkyl” means a cyclic alkyl having from 3 to 7 carbon atoms, such as cyclopropyl, cyclopentyl, cyclohexyl, and the like.

“Cycloalkyloxy” means —O-(cycloalkyl), such as —O-cyclohexyl, and the like.

“Cycloalkylalkyloxy” means —O-(alkyl)-(cycloalkyl, such as —OCH₂cyclohexyl, and the like.

“Alkylidene” means the divalent radical —CHₙH₂ₙ—, wherein n is an integer from 1 to 8, such as —CH₂—, —CH₂CH₂—, —CH₂—CH₂—CH₂—, —CH₂CH₂CH₂CH₂—, —CH₂CH₂CH₂CH₂CH₂—, and the like.

“Heteroatom-containing alkylidene” means an alkylidene wherein at least one carbon atom is replaced by a heteroatom selected from nitrogen, oxygen or sulfur, such as —CH₂CH₂OCH₂CH₂—, and the like.

“Aminoalkoxy” means —O-(alkyl)-NH₂, such as —OCH₂NH₂, —OCH₂CH₂NH₂, and the like.

“Mono- or di-alkylamino” means —NH(alkyl) or —N(alkyl)(alkyl), respectively, such as —NHCH₃, —N(CH₃)₂, and the like.

“Mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

“Arylamino” means —NH(aryl), such as —NH-phenyl, —NH-pyridinyl, and the like.

“Arylalkylamino” means —NH-(alkyl)-(aryl), such as —NH-benzyl, —NHCH₂-pyridinyl, and the like.

“Alkylamino” means —NH(alkyl), such as —NHCH₃, —NHCH₂CH₃, and the like.

“Cycloalkylamino” means —NH-(cycloalkyl), such as —NH-cyclohexyl, and the like.

“Cycloalkylalkylamino”—NH-(alkyl)-(cycloalkyl), such as —NHCH₂-cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH3) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

The term “derivative” as used herein refers to a chemical substance related structurally to another, i.e., an “original” substance, which can be referred to as a “parent” compound. A “derivative” can be made from the structurally-related parent compound in one or more steps. In some embodiments, the general physical and chemical properties of a derivative can be similar to or different from the parent compound.

The term “protected derivatives” means derivatives of compounds described herein in which a reactive site or sites are blocked with protecting groups. Protected derivatives are useful in the preparation of compounds or in themselves can be active. A comprehensive list of suitable protecting groups can be found in T. W. Greene, Protecting Groups in Organic Synthesis, 3rd edition, John Wiley & Sons, Inc. 1999.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

A “pharmaceutically acceptable salt”, as used herein, is intended to encompass any compound described herein that is utilized in the form of a salt thereof, especially where the salt confers on the compound improved pharmacokinetic properties as compared to the free form of compound or a different salt form of the compound. The pharmaceutically acceptable salt form can also initially confer desirable pharmacokinetic properties on the compound that it did not previously possess, and may even positively affect the pharmacodynamics of the compound with respect to its therapeutic activity in the body. An example of a pharmacokinetic property that can be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Pharmaceutically acceptable salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), the content of which is herein incorporated by reference in its entirety. Exemplary salts also include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. Suitable acids which are capable of forming salts with the compounds of the disclosure include inorganic acids such as hydrochloric acid, hydrobromic acid, perchloric acid, nitric acid, thiocyanic acid, sulfuric acid, phosphoric acid, and the like; and organic acids such as 1,2-ethanedisulfonic acid, 2-hydroxyethanesulfonic acid, 2-naphthalenesulfonic acid, 3-phenylpropionic acid, 4-methylbicyclo[2.2.2]oct-2-ene-1-carboxylic acid, 4,4′-mefhylenebis(3-hydroxy-2-ene-1-carboxylic acid), acetic acid, anthranilic acid, benzenesulfonic acid, benzoic acid, camphorsulfonic acid, cinnamic acid, citric acid, cyclopentanepropionic acid, ethanesulfonic acid, formic acid, fumaric acid, glucoheptonic acid, gluconic acid, glutamic acid, glycolic acid, heptanoic acid, hydroxynaphthoic acid, lactic acid, lauryl sulfuric acid, maleic acid, malic acid, malonic acid, mandelic acid, methanesulfonic acid, muconic acid, naphthalene sulfonic acid, o-(4-hydroxybenzoyl)benzoic acid, oxalic acid, p-chlorobenzenesulfonic acid, propionic acid, p-toluenesulfonic acid, pyruvic acid, salicylic acid, stearic acid, succinic acid, sulfanilic acid, tartaric acid, tertiary butylacetic acid, trifluoroacetic acid, trimethylacetic acid, and the like. Suitable bases capable of forming salts with the compounds of the disclosure include inorganic bases such as sodium hydroxide, ammonium hydroxide, sodium carbonate, calcium hydroxide, potassium hydroxide and the like; and organic bases such as mono-, di- and tri-alkyl and aryl amines (e.g., triethylamine, diisopropyl amine, methyl amine, dimethyl amine, N-methylglucamine, pyridine, picoline, dicyclohexylamine, N,N′-dibezylethylenediamine, and the like), and optionally substituted ethanol-amines (e.g., ethanolamine, diethanolamine, trierhanolamine and the like).

In some embodiments, the compounds described herein can be in the form of a prodrug. The term “prodrug” as used herein refers to compounds that can be converted via some chemical or physiological process (e.g., enzymatic processes and metabolic hydrolysis) to compound described herein. Thus, the term “prodrug” also refers to a precursor of a biologically active compound that is pharmaceutically acceptable. A prodrug can be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. For example, a compound comprising a hydroxy group can be administered as an ester that is converted by hydrolysis in vivo to the hydroxy compound. Suitable esters that can be converted in vivo into hydroxy compounds include acetates, citrates, lactates, tartrates, malonates, oxalates, salicylates, propionates, succinates, fumarates, formates, benzoates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group can be administered as an amide, e.g., acetamide, formamide and benzamide that is converted by hydrolysis in vivo to the amine compound. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which are herein incorporated by reference in its entirety.

Synthesis and purification of A N1-methyl-pyrazoloanthrone, e.g., a compound of formula (I)-(VII), such as N1-methyl 1,9-pyrazoloanthrone as disclosed herein can generally be made by organic synthesis techniques known to those skilled in the art, as well as by the methods disclosed in U.S. Pat. No. 7,119,114, which is incorporated herein in its entirety by reference.

Pharmaceutically acceptable salts of compounds of formula (I)-(VII) are also within the scope of this invention. To this end, the compound may generally be utilized as the free base. Alternatively, the compounds may be used in the form of acid addition salts. Acid addition salts of the free base amino compounds of the present invention may be prepared by methods well known in the art, and may be formed from organic and inorganic acids. Suitable organic acids include maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Thus, the term “pharmaceutically acceptable salt” of a compound of structure (I) is intended to encompass any and all acceptable salt forms.

### Method of Treatment of a Subject

The present invention relates generally to a method of treating a proliferative disease or disorder in a subject, where the proliferative disease or disorder is a cancer, e.g., ovarian cancer or other type of cancer which expresses or overexpresses least one kinase of the Casein Kinase I family (e.g., CSNK1A1, CSNK1B, CSNK1G1, CSNKIG2, CSNKIG3, CSNK1D, CSNK1E) and/or ARK5/NUAK1 and/or a MIS receptor. In some embodiments, the cancer also expresses MISRII. In some embodiments, the proliferative disease or disorder is cancer, where the cancer or cancer cells express for example ovarian cancer and other cancer expressing at least one kinase of the Casein Kinase I family (e.g., CSNK1A1, CSNK1B, CSNK1G1, CSNKIG2, CSNKIG3, CSNK1D, CSNK1E) and/or ARK5/NUAK1. The method of the present invention comprises the administration of an effective amount of a compound of formula (I)-(VII), e.g., such as N1-methyl 1,9-pyrazoloanthrone as disclosed herein, or analogue or derivative thereof to a subject in with a proliferative disorder, where the cells associated with the proliferative disorder express or overexpress least one kinase of the Casein Kinase I family (e.g., CSNK1A1, CSNK1B, CSNKIG1, CSNK1G2, CSNK1G3, CSNK1D, CSNK1E) and/or ARK5/NUAK1. For example, an effective amount of compound of formula (I)-(VII), e.g., such as N1-methyl 1,9-pyrazoloanthrone as disclosed herein, or analogue or derivative thereof is administered to a subject that has been selected due to having a cancer that expresses or overexpresses (e.g., has increased expression above a threshold level) of least one kinase of the Casein Kinase I family (e.g., CSNK1A1, CSNK1B, CSNKIG1, CSNK1G2, CSNK1G3, CSNK1D, CSNK1E) and/or ARK5/NUAK1. In alternative embodiments, an effective amount of compound of formula (I)-(VII), e.g., such as N1-methyl 1,9-pyrazoloanthrone as disclosed herein, or analogue or derivative thereof is administered to a subject that has been selected due to the presence of at least one genetic alteration in ARK3/NUAK1 gene and/or at least one genetic mutation in at least one member of the casein kinase 1 family selected from the following genes: CSNK1A1, CSNK1B, CSNK1D, CSNK1E, CSNK1G1, CSNK1G2 or CSNK1G3. Thus, by using the methods of the present invention, one can select a subject to be treated and intervene in the proliferative disease, for example cancer, ameliorate the symptoms, and in some cases cure the disease.

Examples of such diseases where proliferating cells or a cancer expresses at least one kinase of the Casein Kinase I family (e.g., CSNK1A1, CSNK1B, CSNK1G1, CSNK1G2, CSNK1G3, CSNK1D, CSNK1E) and/or ARK5/NUAK1 include, for example, ovarian cancer, cervical cancer, breast cancer, prostate cancer, and endometrial cancer. In some embodiments, the cancer is selected from the group consisting of, for example, ovarian cancer, prostate cancer, bladder cancer, melanoma, pancreatic cancer, sarcoma, liver cancer, stomach cancer, breast cancer, uterine cancer or adenoid cancer. In some embodiments, the cancer is selected from the group consisting of, for example, brain cancer (glioblastoma, medulloblastoma), leukemia (B-ALL, T-ALL or ALL), lung cancer (e.g., lung squamous carcinoma), prostate cancer, renal cancer (e.g., renal cell carcinoma or RCC including clear cell RCC (ccRCC)), colorectal cancer, lymphoma (follicular or large B-cell), mesothelioma. In some embodiments, a cancer expressing or overexpressing (e.g., has increased expression above a threshold level) at least one member of the Casein Kinase I family (e.g., CSNK1A1, CSNK1B, CSNK1G1, CSNK1G2, CSNK1G3, CSNK1D, CSNK1E) and/or ARK5/NUAK1 comprises a cancer stem cell. In some embodiments, such a cancer cell expressing at least one member of the Casein Kinase I family (e.g., CSNK1A1, CSNK1B, CSNK1G1, CSNK1G2, CSNK1G3, CSNK1D, CSNK1E) and/or ARK5/NUAK1 is, for example, an ovarian cancer stem cell, a cervical cancer stem cell, a breast cancer stem cell, a prostate cancer stem cell, endometrial cancer stem cell, a vulvar epidermal carcinoma cancer stem cell, an endometrial edenocarinaoma cancer stem cell, an ovarian adenocarcinoma cancer stem cell. In some embodiments, the cancer can also express at least one MIS receptor, for example MISRII.

Examples of cancers where a subject is selected for treatment with a compound of formula (I)-(VII), such as N1-methyl 1,9-pyrazoloanthrone as disclosed herein according to the method as disclosed herein, or where the subject is selected based on one or more genetic alterations in at least one member of the casein kinase 1 family (e.g., a mutation, SNP or copy number alteration (CNA)) in at least one of the following genes, CSNK1A1, CSNK1B, CSNK1D, CSNK1E, CSNK1G1, CSNK1G2, CSNK1G3) include, for example, ovarian cancer, prostate cancer, bladder cancer, melanoma, pancreatic cancer, sarcoma, liver cancer, stomach cancer, breast cancer, uterine cancer or adenoid cancer. More specifically, a subject with a genetic alteration (e.g., mutation, deletion, SNP or copy number alteration (CNA)) in CSNK1A1 selected for treatment with a composition as disclosed herein has at least one of the following cancers; ccRCC, pancreatic cancer, Bladder cancer, AML, stomach cancer, Sarcoma, uterine cancer, ovarian cancer, melanoma, chRCC, liver cancer, ACC, colorectal cancer, lung adenocarcinoma, head and neck cancer, pRCC, lung squamous cell carcinoma, breast cancer, prostate cancer, thyroid cancer and glioma. In some embodiments, a subject with a genetic alteration (e.g., mutation, deletion, SNP or copy number alteration (CNA)) in CSNK1D selected for treatment with a composition as disclosed herein has at least one of the following cancers; liver cancer, sacrcoma, melanoma, lung adenocarcinoma, ovarian cancer, ACC, lung squamous cell carcinoma (lung sq.), breast cancer, colorectal cancer, bladder cancer, pRCC, uterine cancer, stomach cancer, pancreatic cancer, glioma cancer, head and neck cancer, glioblastoma. In some embodiments, a subject with a genetic alteration (e.g., mutation, deletion, SNP or copy number alteration (CNA)) in CSNK1E selected for treatment with a composition as disclosed herein has at least one of the following cancers; melanoma, liver cancer, stomach cancer, glioma, sacrcoma, pRCC, glioblastoma (GBM).

Examples of cancers where a subject is selected for treatment with a compound of formula (I)-(VII), such as N1-methyl 1,9-pyrazoloanthrone as disclosed herein according to the method as disclosed herein, or where the subject is selected based on at least one genetic alteration (e.g., a SNP or or genetic mutation) in ARK3/NUAK1, include, for example, pancreatic cancer, melanoma, prostate cancer, sarcoma, stomach cancer, lung cancer, uterine cancer, colorectal cancer, colon cancer, esophageal cancer and/or bladder cancer.

In alternative embodiments, a cancer expressing, or having elevated expression of at least one kinase of the Casein Kinase I family (e.g., CSNK1A1, CSNK1B, CSNK1G1, CSNK1G2, CSNK1G3, CSNK1D, CSNK1E) and/or ARK5/NUAK1 includes for example but not limited to; breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, genital-urinary cancer, stomach cancer, lymphomas, melanoma, glioma, bladder cancer, pancreatic cancer, gum cancer, kidney cancer, retinal cancer, liver cancer, nasopharynx cancer, ovarian cancer, oral cancers, bladder cancer, hematological neoplasms, follicular lymphoma, cervical cancer, multiple myeloma, osteosarcomas, thyroid cancer, prostate cancer, colon cancer, prostate cancer, skin cancer, stomach cancer, testis cancer, tongue cancer, or uterine cancer.

In some embodiments, the subject has increased expression of CSNK1A as compared to a pre-defined threshold level, where the increased expression is detected a biological sample obtained from a subject with brain cancer, prostate cancer, lymphoma or leukemia.

In some embodiments, the subject has increased expression of CSNK1D and/or CSNK1E as compared to a pre-defined threshold level, and where the increased expression is detected in a tumor biopsy sample from a subject with bladder cancer, brain cancer, breast cancer, colorectal cancer, kidney cancer, lung cancer, melanoma, ovarian cancer, pancreatic cancer, ductal adenocarcinoma, prostate cancer or a cancer of the hematopoetic system. In some embodiments, the subject has increased expression of CSNK1D as compared to a pre-defined threshold level, where the increased expression is detected a biological sample comprising hyperplastic B cell follicles and/or B cell lymphoma and/or a subject with chriocarcinoma. In some embodiments, the subject has increased expression of CSNK1 G3 as compared to a pre-defined threshold level, where the increased expression is detected a biological sample obtained from a subject with renal cell carcinoma (RCC).

In alternative embodiments, the present invention relates to the use of a N1-methyl-pyrazoloanthrone, e.g., a compound of formula (I)-(VII), such as N1-methyl 1,9-pyrazoloanthrone as disclosed herein and functional derivatives thereof for the treatment of any disorder where inhibition of at least one kinase of the Casein Kinase I family (e.g., CSNK1A1, CSNK1B, CSNK1G1, CSNK1G2, CSNK1G3, CSNK1D, CSNK1E) and/or ARK5/NUAK11 is whole, or part of, the therapeutic regime.

In a related embodiment, a tissue to be treated is a tumor tissue of a subject expressing at least one kinase of the Casein Kinase I family (e.g., CSNK1A1, CSNK1B, CSNK1G1, CSNK1G2, CSNK1G3, CSNK1D, CSNK1E) and/or ARK5/NUAK1, for example the tumor tissue is, but not limited to a solid tumor, a metastases, a skin cancer, a breast cancer, an ovarian cancer, an cervical cancer, a hemangioma or angiofibroma and the like cancer. Typical solid tumor tissues treatable by the pharmaceutical composition of the invention, includes for example, but not limited to tumors of the lung, pancreas, breast, colon, laryngeal, ovarian, and the like tissues. In some embodiment, the solid tumor tissue treatable by the present methods include thyroid, and the cancer type is medullary thyroid cancer.

In some embodiments, the level of expression (e.g., mRNA or protein expression) and/or activity of at least one member of the CK1 family or ARK5/NUAK1 is measured in a biological sample obtained from the subject, such as, for example, a biopsy sample, and if the level of expression is above a predefined threshold level, then the subject is administered a N-methyl pyrazoloathrone compound, e.g., a N1-methyl pyrazoloathrone or derivative thereof. In some embodiments, the predefined threshold level is the level of expression of the CK1 family or ARK5/NUAK1 from a normal or healthy subject without cancer. In some embodiments, if the level is increased above the pre-defined level, e.g., about 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level, then the subject is administered a compound of formula (I)-(VII) as disclosed herein, e.g., a N-methyl pyrazoloathrone compound such as a N1-methyl pyrazoloathrone or derivative thereof.

In some embodiments, a subject is selected to be administered a N-methyl pyrazoloathrone compound such as a N1-methyl pyrazoloathrone or derivative thereof if: (a) a biological sample obtained from the subject has increased mRNA or protein expression of at least one member of the Casein Kinase I family or ARK5/NUAK1 kinase; or (b) the subject with cancer is determined to have at one genetic alteration in the ARK3/NUAK1 gene and/or at least one genetic mutation in at least one member of the Casein Kinase 1 family. In some embodiments, the present invention encompasses a step of (a) measuring the level of protein or mRNA expression of at least one member of the Casein Kinase I family or ARK5/NUAK1 kinase in a biological sample obtained from the subject, and/or (b) determining if the subject has at least one genetic alteration in ARK3/NUAK1 gene and/or at least one genetic mutation in at least one member of the casein kinase 1 family.

In such embodiments, the predefined threshold level of expression or activity of a member of CK1 and/or ARK5/NUAK1 is the level of expression (or activity) of the member of the CK1 family or ARK5/NUAK1 present in a comparative biological sample from a normal or healthy subject without cancer. In some embodiments, if the expression level (or activity level) is increased above the pre-defined level, e.g., about 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level, then the subject is administered a compound of formula (I)-(VII) as disclosed herein, e.g., a N-methyl pyrazoloathrone compound such as a N1-methyl pyrazoloathrone or derivative thereof.

By way of example only, in some embodiments, a subject selected for administration of the compositions as disclosed herein has high levels or increased expression above a predefined threshold level of (i) CSNK1A in lung cancer, colon cancer, and liposarcoma and/or CSNK1D in lung cancer or glioblastoma, and/or (iii) a CSNK1E in breast cancer, and lun cancer, or glioma such as high-grade glioma.

Methods to measure the protein or mRNA expression level of ARK3/NUAK1 or a least one member of the casein kinase 1 family (e.g., CSNK1A1, CSNK1B, CSNK1D, CSNK1E, CSNK1G1, CSNK1 G2 or CSNK1G3) of are well known in the art, and include, for example, RT-PCR, detection of protein levels using antibody binding or antibody staining methods, western blot analysis, ELISA, and the other protein-detection methods known to one of ordinary skill in the art.

In some embodiments, the present invention relates to method of treating a subject with cancer, comprising: (a) assessing the expression and/or activity of at least one member of the Casein Kinase I family or ARK5/NUAK1 kinase in a biological sample obtained from the subject, and/or (b) determining if the subject has at least one genetic alteration in ARK3/NUAK1 gene; and/or determining if the subject has at least one genetic alteration in at least one member of the casein kinase 1 family selected from the following genes: CSNK1A1, CSNK1B, CSNK1D, CSNK1E, CSNK1G1, CSNK1G2 or CSNK1G3; and selecting the subject to be administered a composition comprising a compound of formula (I)-(VII) as disclosed herein, e.g., a N-methyl pyrazoloathrone compound such as a N1-methyl pyrazoloathrone or derivative thereof, if the subject has increased expression and/or activity of at least one member of the Casein Kinase I family or ARK5/NUAK1 kinase above a predefined threshold level, and/or at least one genetic alteration in ARK3/NUAK1 gene; and/or the subject has at least one genetic alteration in at least one member of the casein kinase 1 family selected from the following genes: CSNK1A1, CSNK1B, CSNK1D, CSNK1E, CSNK1G1, CSNK1G2 or CSNK1G3.

In some embodiments, a subject is selected for administration of a a composition comprising a compound of formula (I)-(VII) as disclosed herein, e.g., a N-methyl pyrazoloathrone compound such as a N1-methyl pyrazoloathrone or derivative thereof due to a genetic alteration in at least one member of the CK1 family. Such genetic alterations are known to one of ordinary skill in the art, and are disclosed in Schittek et al., Mol. Cancer, 2014; 13; 231 (“Biological functions of casein kinase 1 isoforms and putative roles in tumorigenesis”), and Knippschild et al., Front. Oncol., 2014; 4; 96 (“The CK1 family: contribution to cellular stress response and its role in carcinogensis), both of which are incorporated herein in its entirety by reference. By way of example only, in some embodiments, a subject selected for administration of the compositions as disclosed herein has a R324H mutation in CSNK1D.

Methods to detect a genetic alteration in a least one member of the Casein Kinase I family (e.g., CSNK1A1, CSNK1B, CSNK1G1, CSNK1G2, CSNK1G3, CSNK1D, CSNK1E) or ARK5/NUAK1 kinase are also well known in the art, and include RT-PCR, hybridization methods, direct sequencing etc.

The presence of at least one kinase of the CK1 family (e.g., CSNK1A1, CSNK1B, CSNK1G1, CSNK1G2, CSNK1G3, CSNK1D, CSNK1E) and/or ARK5/NUAK1 in fluids such as blood or a biopsy sample obtained from the subject may be indicative of the presence of cancer. In some embodiments, the biological sample obtained from the subject is a cancer or tumor tissue sample or a cancer cell or tumor cell or a blood or plasma sample. In some embodiments, the biological sample is a biopsy tissue sample. The presence of increased expression of at least one kinase of the CK1 family (e.g., CSNK1A1, CSNK1B, CSNK1G1, CSNK1G2, CSNK1G3, CSNK1D, CSNK1E) and/or ARK5/NUAK1 in fluids or sites not near a tumor may also be indicative of metastasis, and can be used to select a subject for administration with a composition comprising a compound of formula (I)-(VII) as disclosed herein, e.g., a N-methyl pyrazoloathrone compound such as a N1-methyl pyrazoloathrone or derivative thereof as disclosed herein.

In some such embodiments, the compounds of the present invention can be administered to the subject, and in some embodiments the compounds of the present invention are administered to the subject in a pharmaceutical composition comprising one or more additional therapies. In all aspects of the present invention, a N-methyl pyrazoloanthrone or derivative or analogue thereof, e.g., N1-methyl-1,9-pyrazoloathrone can be administered alone, or in combination with additional agents, such as chemotherapeutic agents, such as hMIS or a variant thereof, such as a LR-MIS variant comprising the amino acids of SEQ ID NO: 1. In some embodiments, a compound of formula (I)-(VII), e.g., such as N1-methyl 1,9-pyrazoloanthrone as disclosed herein or analogue thereof, is administered in combination with an anti-cancer agent, or hMIS protein or a MIS variant, where the hMIS protein or LR-MIS variant protein can be expressed by a viral vector, e.g., adenoviral vector, a poxvirus vector, or lentiviral vector, or AAV vector such as AAV9.

In a related embodiment, the invention contemplates the practice of the method in conjunction with other therapies such as conventional chemotherapy directed against solid tumors and for control of establishment of metastases. The administration of the compounds described herein is typically conducted prior to and/or at the same time and/or after chemotherapy, although it is also encompassed within the present invention to inhibit cell proliferation after a regimen of chemotherapy at times where the tumor tissue will be responding to the toxic assault by inducing angiogenesis to recover by the provision of a blood supply and nutrients to the tumor tissue. In addition, the pharmaceutical compositions of the invention for the treatment of proliferative disorders, for example cancer, can be administrated prophylactically and/or before the development of a tumor, if the subject has been identified as to have a risk of developing cancer, for example to subjects that are positive for biomarkers of cancer cells or tumors. Insofar as the present methods apply to inhibition of cell proliferation, the methods can also apply to inhibition of tumor tissue growth, to inhibition of tumor metastases formation, and to regression of established tumors.

The inventive methods disclosed herein provide for the parenteral and oral administration of the compounds of the present invention, e.g., a N1-methyl-pyrazoloanthrone, e.g., a compound of formula (I)-(VII), such as N1-methyl 1,9-pyrazoloanthrone as disclosed herein in combination with other pharmaceutical compositions to subjects in need of such treatment. Parenteral administration includes, but is not limited to, intravenous (IV), intramuscular (IM), subcutaneous (SC), intraperitoneal (IP), intranasal, and inhalant routes. In the method of the present invention, the resolvins and/or protectins or analogs thereof are preferably administered orally. IV, IM, SC, and IP administration may be by bolus or infusion, and may also be by slow release implantable device, including, but not limited to pumps, slow release formulations, and mechanical devices. The formulation, route and method of administration, and dosage will depend on the disorder to be treated and the medical history of the subject. In general, a dose that is administered by subcutaneous injection will be greater than the therapeutically-equivalent dose given intravenously or intramuscularly. Preferably, the dose of a N1-methyl-pyrazoloanthrone, e.g., a compound of formula (I)-(VII), such as N1-methyl 1,9-pyrazoloanthrone as disclosed herein will be administered at doses from about 0.1 mg to about 250 mg of body weight. In some embodiments, a unit dose of a compound of the present invention will be from about 1 mg to about 60 mg. In some embodiments, a unit dose of a N1-methyl-pyrazoloanthrone, e.g., a compound of formula (I)-(VII), such as N1-methyl 1,9-pyrazoloanthrone (which was used in mice at 30 mg/kg, 60 mg/kg and 600 mg/kg) is between about between 2.5-5.0 mg/kg, or between about 1.0-10.0 mg/kg, or between about 2.0-5.0 mg/kg, or between about 1.0-2.5 mg/kg, or between about 2.0-7.5 mg/kg. In some embodiments, a unit dose is about 2.5 mg/kg. In some embodiments, a unit dose is about 5 mg/kg. In some embodiments, a unit dose for a human subject is about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or anywhere between 0.5 mg/kg and 2.0 mg/kg, or anywhere between 2.0 mg/kg and 4.0 mg/kg, or anywhere between 4.0 mg/kg and 6.0 mg/kg, or anywhere between 6.0 mg/kg and 8.0 mg/kg, or anywhere between 8.0 mg/kg and 10.0 mg/kg, or anywhere between 10.0 mg/kg and 20 mg/mg, or anywhere between 20 mg/kg and 30 mg/kg, or anywhere between about 30 mg/kg and 40 mg/kg, or anywhere between about 40 mg/kg and 50 mg/kg, or anywhere between about 50 mg/kg and 60 mg/kg.

The methods of the present invention for treating cancer expressing or overexpressing at least one kinase of the CK1 family (e.g., CSNK1A1, CSNK1B, CSNK1G1, CSNK1G2, CSNK1G3, CSNK1D, CSNK1E) and/or ARK5/NUAK1 are useful for treatment of proliferation-related diseases or cancer, comprising contacting a tissue in which proliferation is occurring, or is at risk for occurring, with the compositions of the present invention comprising a therapeutically effective amount of a N1-methyl-pyrazoloanthrone, e.g., a compound of formula (I)-(VII), such as N1-methyl 1,9-pyrazoloanthrone as disclosed herein or functional derivatives thereof.

Other conditions which can be treated with a N-methyl pyrazoloanthrone or derivative or analogue thereof, e.g., N1-methyl-1,9-pyrazoloathrone or benefit from MIS or activation of MIS signaling includes cancers which expresses at least one member of the CK1 family and/or ARK5/NUAK1, as discussed above.

Furthermore, in other embodiments, indications which can be treated with a N-methyl pyrazoloanthrone or derivative or analogue thereof, e.g., N1-methyl-1,9-pyrazoloathrone are conditions where excess androgen is present, for example, but not limited to, rheumatoid arthritis, proliferative diseases such as cancer, treatment of prostatic cancer, polycysic ovarian disease, benign prostatic hypertrophy and precocious puberty and other hyperandrogen disorders such as testitoxicosis.

In some embodiments, a N-methyl pyrazoloanthrone or derivative or analogue thereof, e.g., N1-methyl-1,9-pyrazoloathrone can be used as a method of contraception in a female subject and/or to protect ovarian reserves in a female subject. In some embodiments, the subject is a human subject, e.g., a female human, however, where N1-methyl-1,9-pyrazoloathrone is being used to protect ovarian reserve or as a female contraceptive, the female is a female mammal, e.g., domestic animals (e.g., dog, cat etc.) as well as feral or wild animals (e.g. feral cats, feral dogs), and other non-human mammals, for example, selected from the group consisting of; mouse, rat, rabbit, cow, horse, pig, chicken, dog, cat, macaque, chimpanzee, or a domestic or commercial animals such as a cow, pig, horse, deer, bison, llama, mule, rabbit, reindeer, sheep, water buffalo, yak, poultry, fish and other livestock raised in an agricultural setting for commodities such as food, fiber and labor.

In some embodiments, the subject treated by the methods of the present invention in its many embodiments is a human subject, although it is to be understood that the principles of the invention indicate that the invention is effective with respect to all mammals. In this context, a mammal is understood to include any mammalian species in which treatment of diseases associated with cancer or a proliferative-related disorder is desirable, or where protection of the ovarian reserve or contraception is desirable, particularly with respect to agricultural and domestic mammalian species, as well as transgenic animals.

### Administration of Pharmaceutical Compositions

A physician or veterinarian having ordinary skill in the art can readily determine and prescribe the effective amount of the pharmaceutical composition of a N1-methyl-pyrazoloanthrone, e.g., a compound of formula (I)-(VII), such as N1-methyl 1,9-pyrazoloanthrone as disclosed herein which is required. For example, the physician or veterinarian could start doses of the compounds of the invention employed in the pharmaceutical composition at levels lower than that required in order to achieve the desired therapeutic effect and gradually increase the dosage until the desired effect is achieved.

A compound of formula (I)-(VII), such as N1-methyl 1,9-pyrazoloanthrone as disclosed herein can be formulated with an appropriate pharmaceutically acceptable carrier in a desired dosage and such a pharmaceutical composition can be administered to a subject. A pharmaceutical composition as disclosed herein can be administered to a subject using any suitable means. In general, suitable means of administration include, but are not limited to, topical, oral, parenteral (e.g., intravenous, subcutaneous or intramuscular), rectal, intracisternal, intravaginal, intraperitoneal, ocular, or nasal routes.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

When a compound of the present invention, for example a N1-methyl-pyrazoloanthrone, e.g., a compound of formula (I)-(VII), such as N1-methyl 1,9-pyrazoloanthrone as disclosed herein is administered as a pharmaceutical to humans and mammals, they can be given per se or as a pharmaceutical composition containing, for example, 0.1 to 99.5% (more preferably, 0.5 to 90%) of active ingredient, i.e., at least one compound of formula (I)-(VII), such as N1-methyl 1,9-pyrazoloanthrone as disclosed herein and/or derivative thereof, in combination with a pharmaceutically acceptable carrier.

The term “dosage unit” or “unit dose” as used herein refers to physically discrete units suited as unitary dosages for the mammalian subjects to be treated; each unit containing a predetermined quantity of active compound calculated to produce the desired therapeutic effect in association with the required pharmaceutical carrier. A dosage unit of compounds of the invention are dictated by and directly dependent on (a) the unique characteristics of the compound, a N1-methyl-pyrazoloanthrone, e.g., a compound of formula (I)-(VII), such as N1-methyl 1,9-pyrazoloanthrone and/or derivative thereof and the particular therapeutic or prophylactic effect to be achieved, and (b) the limitations inherent in the art of compounding such an active compound for the treatment of sensitivity in individuals. Typically, unit doses are the effective amount of the composition or compound for a desired therapeutic effect (e.g., reduction of a tumor size, protecting ovarian reserve etc.). Unit doses can be administered in sub-doses (e.g., by pulsed administration), or as a bolus administration. The pulsed administration or bolus administration of a unit dose can be administered to the subject in a defined period of time followed by a period of no administration of the compound.

Dosage regimens may be adjusted to provide the optimum desired response (e.g. a therapeutic or prophylactic response). For example, a single bolus may be administered or a dose may be divided into sub-doses (e.g., pulsed administration), and in either situation, the bolus or pulsed administration may be administered over a defined period of time, and in some instances, the unit dose may be proportionally reduced or increased as indicated by the exigency of the therapeutic situation. It is especially advantageous to formulate parenteral compositions in dosage unit form for ease of administration and uniformity of dosage. Actual dosage levels of the active ingredients in the pharmaceutical compositions comprising one or more N1-methyl-pyrazoloanthrone, e.g., a compound of formula (I)-(VII), such as N1-methyl 1,9-pyrazoloanthrone may be varied so as to obtain an amount of the active ingredient which is effective to achieve the desired therapeutic response for a particular subject, composition, and mode of administration, without being toxic to the patient.

In some embodiments, a subject can receive a composition comprising a N1-methyl-pyrazoloanthrone, e.g., N1-methyl 1,9-pyrazoloanthrone as disclosed herein for life or for a defined period of time, for example, where the N1-methyl-pyrazoloanthrone, e.g., N1-methyl 1,9-pyrazoloanthrone is being used as a contraceptive, or for protecting ovarian reserves, or it is desirable to prevent the subject from reproducing or getting pregnant.

Pharmaceutical compositions comprising a N1-methyl-pyrazoloanthrone, e.g., a compound of formula (I)-(VII), such as N1-methyl 1,9-pyrazoloanthrone as disclosed herein can include a “therapeutically effective amount” or a “prophylactically effective amount” of one or more of the compounds of the present invention, or functional derivatives thereof. An “effective amount” is the amount as defined herein in the definition section and refers to an amount effective, at dosages and for periods of time necessary, to achieve the desired therapeutic result, e.g., a diminishment or prevention of effects associated with proliferative disease states or conditions, such as cancer, wherein the cancer expresses or overexpresses (e.g., expresses high levels of) at least one kinase of the CK1 family (e.g., CSNK1A1, CSNK1B, CSNK1G1, CSNK1G2, CSNK1G3, CSNK1D, CSNK1E) and/or ARK5/NUAK1. A therapeutically effective amount of N1-methyl-pyrazoloanthrone, e.g., a compound of formula (I)-(VII), such as N1-methyl 1,9-pyrazoloanthrone as disclosed herein or functional derivatives thereof may vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to, or at an earlier stage of disease, the prophylactically effective amount may be less than the therapeutically effective amount. A prophylactically or therapeutically effective amount is also one in which any toxic or detrimental effects of the compound are outweighed by the beneficial effects.

In some embodiments, therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount of a N1-methyl-pyrazoloanthrone, e.g., a compound of formula (I)-(VII), such as N1-methyl 1,9-pyrazoloanthrone as disclosed herein is sufficient to reduce or inhibit cell proliferation in a subject suffering from a proliferative disorder, for example cancer, such as a cancer expressing at least one kinase of the CK1 family (e.g., CSNK1A1, CSNK1B, CSNK1G1, CSNK1G2, CSNK1G3, CSNK1D, CSNK1E) and/or ARK5/NUAK. In some embodiments, the therapeutically effective amount is sufficient to eliminate the proliferative cells, for example eliminate the cancer cells and/or tumor in a subject suffering cancer and/or a proliferative disease.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of a N1-methyl-pyrazoloanthrone, e.g., a compound of formula (I)-(VII), such as N1-methyl 1,9-pyrazoloanthrone as disclosed herein or functional derivatives thereof, and the condition of the patient, as well as the body weight or surface area of the patient to be treated. The size of the dose is also determined by the existence, nature, and extent of any adverse side-effects that accompany the administration of a particular vector, formulation, or the like in a particular subject. Therapeutic compositions comprising one or more N1-methyl-pyrazoloanthrones, e.g., one or more compounds of formula (I)-(VII), including but not limited to such as N1-methyl 1,9-pyrazoloanthrone as disclosed herein or functional derivatives thereof are optionally tested in one or more appropriate in vitro and/or in vivo animal models of disease, such as models of cancer, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of a N1-methyl-pyrazoloanthrone, e.g., a compound of formula (I)-(VII), such as N1-methyl 1,9-pyrazoloanthrone as disclosed herein or functional derivatives thereof at various concentrations, e.g., as applied to the mass and overall health of the patient. Administration can be accomplished via single or divided doses.

In vitro models can be used to determine the effective doses of a N1-methyl-pyrazoloanthrone, e.g., a compound of formula (I)-(VII), such as N1-methyl 1,9-pyrazoloanthrone as disclosed herein or functional derivatives thereof as a potential cancer treatment. Suitable in vitro models include, but are not limited to, proliferation assays of cultured tumor cells, growth of cultured tumor cells in soft agar (see Freshney, (1987) Culture of Animal Cells: A Manual of Basic Technique, Wily-Liss, New York, N.Y. Ch 18 and Ch 21), tumor systems in nude mice as described in Giovanella et al., I J. Natl. Can. Inst., 52: 921-30 (1974), mobility and invasive potential of tumor cells in Boyden Chamber assays as described in Pilkington et al., Anticancer Res., 17: 4107-9 (1997), and angiogenesis assays such as induction of vascularization of the chick chorioallantoic membrane or induction of vascular endothelial cell migration as described in Ribatta et al., Intl. J. Dev. Biol., 40: 1189-97 (1999) and Li et al., Clin. Exp. Metastasis, 17:423-9 (1999), respectively. Suitable tumor cells lines are available, e.g. from American Type Tissue Culture Collection catalogs.

In vivo models are the preferred models to determine an effective dose of a N1-methyl-pyrazoloanthrone, e.g., a compound of formula (I)-(VII), such as N1-methyl 1,9-pyrazoloanthrone as disclosed herein or functional derivatives thereof as disclosed herein as potential cancer treatments. Suitable in vivo models include, but are not limited to, mice that carry a mutation in the KRAS oncogene (Lox-Stop-Lox K-RasGi2D mutants, Kras24TYj) available from the National Cancer Institute (NCI) Frederick Mouse Repository. Other mouse models known in the art and that are available include but are not limited to models for breast cancer, gastrointestinal cancer, hematopoietic cancer, lung cancer, mammary gland cancer, nervous system cancer, ovarian cancer, prostate cancer, skin cancer, cervical cancer, oral cancer, and sarcoma cancer (see http://emice.nci.nih. gov/mouse_models/).

In determining the effective amount of a N1-methyl-pyrazoloanthrone, e.g., a compound of formula (I)-(VII), such as N1-methyl 1,9-pyrazoloanthrone as disclosed herein or a functional derivative thereof to be administered in the treatment or prophylaxis of disease the physician evaluates circulating plasma levels, formulation toxicities, and progression of the disease.

The efficacy and toxicity of the compound can be determined by standard pharmaceutical procedures in cell cultures or experimental animals, e.g., ED50 (the dose is effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. In some embodiments, the compounds of the present invention have an ED₅₀ value ranging from 0.01-10M in an assay for inhibition of at least one kinase of the CK1 family (e.g., CSNK1A1, CSNK1B, CSNK1G1, CSNK1G2, CSNK1G3, CSNK1D, CSNK1E) and/or ARK5/NUAK1. Such assays are well known in the art, for example, as disclosed in Rena et al., EMBO Rep. 2004; 5(1): 60-65 (D4476, a cell-permeable inhibitor of CK1, suppresses the site-specific phosphorylation and nuclear exclusion of FOXO1a), which is incorporated herein in its entirety by reference.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

In general, a suitable unit dose of a N1-methyl-pyrazoloanthrone compound, e.g., a compound of formula (I)-(VII), such as N1-methyl 1,9-pyrazoloanthrone as disclosed herein will be the amount of the compound which is the lowest amount to produce a desired therapeutic effect (e.g., reduction of a tumor size, protecting ovarian reserve etc.). Such a unit dose will generally depend upon the factors described above. Generally, intravenous and subcutaneous unit doses of a compound of formula (I)-(VII), such as N1-methyl 1,9-pyrazoloanthrone as disclosed herein for a subject (e.g., a human patient), will range from about 0.1 mg to about 250 mg per kilogram of body weight, more preferably from about 1 mg to about 60 mg per kg. In some embodiments, a unit dose is the amount which is administered in a single day, and in some embodiments, the unit dose is administered over a defined time period (e.g., pulsed administration or continued administration) over 2 days, 3 days, 4 days, 5 days, 1 week, 2 weeks or more than 2 weeks.

An exemplary, non-limiting range for a therapeutically or prophylactically effective amount of a compound of formula (I)-(VII), such as N1-methyl 1,9-pyrazoloanthrone as disclosed herein and/or functional derivatives thereof of the invention is 0.1-250 mg/kg, and in some embodiments, the dosage is between 1 and 60 mg/kg. In some embodiments, the dose of a N1-methyl-pyrazoloanthrone, e.g., a compound of formula (I)-(VII), such as N1-methyl 1,9-pyrazoloanthrone as disclosed herein is between 30-600 mg/kg, or between about 10-1000 mg/kg, or between about 50-500 mg/kg, or between about 100-100 mg/kg, or between about 30-100 mg/kg. In some embodiments, the dose is about 30 mg/kg. In some embodiments, the dose is about 600 mg/kg. In some embodiments, the human equivalent dose (HED) of a N1-methyl-pyrazoloanthrone, e.g., a compound of formula (I)-(VII), such as N1-methyl 1,9-pyrazoloanthrone (which was used in mice at 30 mg/kg, 60 mg/kg and 600 mg/kg) is between about between 2.5-5.0 mg/kg, or between about 1.0-10.0 mg/kg, or between about 2.0-5.0 mg/kg, or between about 1.0-2.5 mg/kg, or between about 2.0-7.5 mg/kg. In some embodiments, a unit dose is about 2.5 mg/kg. In some embodiments, a unit dose is about 5 mg/kg. In some embodiments, a unit dose for a human subject is about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or anywhere between 0.5 mg/kg and 2.0 mg/kg, or anywhere between 2.0 mg/kg and 4.0 mg/kg, or anywhere between 4.0 mg/kg and 6.0 mg/kg, or anywhere between 6.0 mg/kg and 8.0 mg/kg, or anywhere between 8.0 mg/kg and 10.0 mg/kg, or anywhere between 10.0 mg/kg and 20 mg/mg, or anywhere between 20 mg/kg and 30 mg/kg, or anywhere between about 30 mg/kg and 40 mg/kg, or anywhere between about 40 mg/kg and 50 mg/kg, or anywhere between about 50 mg/kg and 60 mg/kg

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition

If desired, a unit dose of a N1-methyl-pyrazoloanthrone, e.g., a compound of formula (I)-(VII), such as N1-methyl 1,9-pyrazoloanthrone as disclosed herein may be administered in a series of pulses, e.g., as two, three, four, five, six or more sub-doses (of the unit dose) administered separately at appropriate intervals throughout the day.

Administration can be by parenteral or nonparenteral means, but in some embodiments, administration is oral or intravenous. Treatment may be for short periods of time, e.g., pulsed or continuous throughout the lifetime of the patient. In some embodiments, administration of a compound of formula (I)-(VII), such as N1-methyl 1,9-pyrazoloanthrone as disclosed herein is a pulsed administration of a unit dose. In certain embodiments, a pulsed administration comprises administering a N1-methyl-pyrazoloanthrone, e.g., a compound of formula (I)-(VII), such as N1-methyl 1,9-pyrazoloanthrone for about 8 weeks, followed by not administering a N1-methyl-pyrazoloanthrone, e.g., N1-methyl 1,9-pyrazoloanthrone as disclosed herein for about 4 weeks. In some embodiments, the pulsed administration comprises administering at least one N1-methyl-pyrazoloanthrone, e.g., N1-methyl 1,9-pyrazoloanthrone as disclosed herein for about 6 weeks, followed by not administering the agent for about 2 weeks. In certain embodiments, the pulsed administration comprises administering at least one N1-methyl-pyrazoloanthrone, e.g., N1-methyl 1,9-pyrazoloanthrone as disclosed herein for about 4 weeks, followed by not administering the agent for about 2 weeks. In some embodiments, the pulsed administration comprises administering at least one N1-methyl-pyrazoloanthrone, e.g., N1-methyl 1,9-pyrazoloanthrone as disclosed herein for about 2 weeks, followed by not administering it for about 2 weeks. In some embodiments, pulsed administration comprises pulses of administering at least a N1-methyl-pyrazoloanthrone, e.g., N1-methyl 1,9-pyrazoloanthrone as disclosed herein for about 1 day, about 2 days, about 3 days, about 4 days, about 5 days, about 6 days, about 7 days, about 10 days, about 2 weeks, about 3 weeks, about 4 weeks, about 5 weeks, about 6 weeks, about 7 weeks, about 8 weeks, about 2 months, about 3 months, about 4 months, about 5 months, about 6 months, about 9 months, about 12 months. In certain embodiments, pulsed administration comprises intervals of not administering a N1-methyl-pyrazoloanthrone, e.g., N1-methyl 1,9-pyrazoloanthrone as disclosed herein of about 1 day, about 2 days, about 3 days, about 4 days, about 5 days, about 6 days, about 7 days, about 10 days, about 2 weeks, about 3 weeks, about 4 weeks, about 5 weeks, about 6 weeks, about 7 weeks, about 8 weeks, about 2 months, about 3 months, about 4 months, about 5 months, about 6 months, about 9 months, about 12 months. In some embodiments, administration is continuous. In certain embodiments, administration is for the lifetime of the subject.

In traditional forms of therapy, repeated administration is designed to maintain a desired level of an active ingredient in the body. Very often, complications that develop can be attributed to dosage levels that, to be effective, are near toxic or otherwise harmful to normal cells. In contrast, with pulse therapy, in vivo levels of drug drop below that level required for effective continuous treatment. Therefore, pulsing is not simply the administration of a sufficiently large bolus such that there will be therapeutically sufficient drug available for a long period of time. Pulsed administration of a unit dose can substantially reduce the amount of the composition administered to the patient per administration dose or per total treatment regimen with an increased effectiveness. This represents a significant saving in time, effort and expense and, more importantly, a lower effective dose substantially lessens the risk of side effects (e.g. Adverse side effects) or the number and severity of complications that may be experienced as a result of the compound by one or more patients.

Individual pulses of a N1-methyl-pyrazoloanthrone, e.g., N1-methyl 1,9-pyrazoloanthrone as disclosed herein as disclosed herein can be delivered to the patient continuously over a period of several hours, such as about 2, 4, 6, 8, 10, 12, 14 or 16 hours, or several days, such as 2, 3, 4, 5, 6, or 7 days, preferably from about 1 hour to about 24 hours and more preferably from about 3 hours to about 9 hours. Alternatively, periodic doses can be administered in a single bolus or a small number of injections of the composition over a short period of time, typically less than 1 or 2 hours. For example, a N1-methyl-pyrazoloanthrone, e.g., N1-methyl 1,9-pyrazoloanthrone as disclosed herein can be administered over a period of 4 days with infusions for about 8 hours per day or overnight, followed by a period of 7 days of no treatment.

In some embodiments, the interval between pulsed administration (e.g., the interval of no drug delivery) is greater than 24 hours and preferably greater than 48 hours, and can be for even longer such as for 3, 4, 5, 6, 7, 8, 9 or 10 days, two, three or four weeks or even longer. Often, the interval between pulsed administration can be calculated by administering another dose of the composition when the composition or the active component of the composition is no longer detectable in the patient prior to delivery of the next pulse. Intervals can also be calculated from the in vivo half-life of the composition. Intervals may be calculated as greater than the in vivo half-life, or 2, 3, 4, 5 and even 10 times greater the composition half-life. For compositions with fairly rapid half lives, intervals may be 25, 50, 100, 150, 200, 250 300 and even 500 times the half life of the chemical composition. The number of pulses in a single therapeutic regimen (e.g., a unit dose) may be as little as two, but is typically from about 5 to 10, 10 to 20, 15 to 30 or more. In some embodiments, a subject can receive pulse administration of the compound for life according to the methods of this invention without the problems and inconveniences associated with current therapies or other chemotherapeutic agents.

In certain embodiments, compositions comprising a N1-methyl-pyrazoloanthrone, e.g., N1-methyl 1,9-pyrazoloanthrone as disclosed herein are administered by most any means, but are preferable delivered to the patient as an injection (e.g. intravenous, subcutaneous, intraarterial), infusion or instillation, and more preferably by oral ingestion.

Pulsed administration of one or more pharmaceutical compositions comprising a N1-methyl-pyrazoloanthrone, e.g., N1-methyl 1,9-pyrazoloanthrone as disclosed herein in a subject, e.g., for treatment of a cancer expressing MIS, or member of the CK1 family or ARK5/NUARK1. In some embodiments, pulsed administration of one or more pharmaceutical compositions comprising a N1-methyl-pyrazoloanthrone, e.g., N1-methyl 1,9-pyrazoloanthrone as disclosed herein can be used for as a female contraceptive, and/or to protect ovarian reserve in a mammalian female subject, such as a human female or a domestic mammal or wild animal. Similarly, pulsed administration of one or more pharmaceutical compositions comprising a N1-methyl-pyrazoloanthrone, e.g., N1-methyl 1,9-pyrazoloanthrone as disclosed herein can be used for prophylactic treatment, e.g., for example, a subject who will, or has or is currently undergoing chemotherapy and chemoradiation therapy. In some embodiments, pulsed administration can be more effective than continuous treatment as pulsed doses (e.g., pulsed administration of a unit dose) results in an overall lower amount of compound used than would be expected from continuous administration of the same composition. Each pulse dose can be reduced and the total amount of drug (e.g., N1-methyl-pyrazoloanthrone, e.g., N1-methyl 1,9-pyrazoloanthrone as disclosed herein) administered over the course of treatment to the patient can be minimized. Similarly, the pulsed administration of N1-methyl-pyrazoloanthrone, e.g., N1-methyl 1,9-pyrazoloanthrone as disclosed herein can be used in combination with pulse or continuous administration of another chemotherapeutic agents, e.g., hMIS or MIS variant (e.g., LR-MIS comprising SEQ ID NO: 1 or a homologue or variant thereof), or other chemotherapeutic such as cisplatin, doxorubicin and paclitaxel, vermurafib etc.

With pulse therapy, in vivo levels of a N1-methyl-pyrazoloanthrone, e.g., N1-methyl 1,9-pyrazoloanthrone as disclosed herein thereof can drop below that level required for effective continuous treatment. Pulsed administration can reduce the amount of a composition comprising a N1-methyl-pyrazoloanthrone, e.g., N1-methyl 1,9-pyrazoloanthrone as disclosed herein thereof administered to the patient per dose, and/or per total treatment regimen with an increased effectiveness. Pulsed administration can provide a saving in time, effort and expense and a lower effective dose can lessen the number and severity of complications that can be experienced by a subject. As such, pulsing can be more effective than continuous administration of the same composition.

In some embodiments, individual pulses can be delivered to a subject continuously over a period of several hours, such as about 2, 4, 6, 8, 10, 12, 14 or 16 hours, or several days, such as 2, 3, 4, 5, 6, or 7 days, or from about 1 hour to about 24 hours or from about 3 hours to about 9 hours. Alternatively, periodic doses of the composition comprising a N1-methyl-pyrazoloanthrone, e.g., N1-methyl 1,9-pyrazoloanthrone as disclosed herein can be administered in a single bolus or a small number of injections (or via other routes) over a short period of time, for example, less than 1 or 2 hours.

In some embodiments, the interval between pulses or the interval of no delivery can be greater than 24 hours or can be greater than 48 hours, and can be for even longer such as for 3, 4, 5, 6, 7, 8, 9 or 10 days, two, three or four weeks or even longer. The interval between pulses can be determined by one of ordinary skill in the art, for example, by measuring the presence of the compound in the blood in the subject after administration of the pulse dose, and administering a pulse when the level of the compound decreases to a certain pre-defined low threshold limit. Such pre-defined low threshold limits can be determined by one of ordinary skill in the art, and can be, for example, about baseline level, or about 80% or about 60% or about 40% or about 20% or less than 20% below the baseline level of the compound after it is immediately administered. Alternatively, in some embodiments, the interval between pulses can be calculated by administering another dose of a composition comprising a N1-methyl-pyrazoloanthrone, e.g., N1-methyl 1,9-pyrazoloanthrone as disclosed herein, and when the active component of the composition is no longer detectable in the patient prior to delivery of the next pulse. Alternatively, intervals can also be calculated from the in vivo half-life of the N1-methyl-pyrazoloanthrone, e.g., N1-methyl 1,9-pyrazoloanthrone as disclosed herein. Compositions can be administered by most any means, and can be delivered to the subject as an oral formulation, or injection (e.g. intravenous, subcutaneous, intraarterial), infusion or instillation. Various methods and apparatus for pulsing compositions by infusion or other forms of delivery to the patient are disclosed in U.S. Pat. Nos. 4,747,825; 4,723,958; 4,948,592; 4,965,251 and 5,403,590, which are incorporated herein in their entirety by reference.

In some embodiments, the number of pulses in a single therapeutic regimen (e.g., single unit dose) can be as little as two, but can be from about 5 to 10, 10 to 20, 15 to 30 or more.

In one embodiment, a composition comprising a N1-methyl-pyrazoloanthrone, e.g., N1-methyl 1,9-pyrazoloanthrone as disclosed herein thereof can be administered to a subject for about 2, or about 3, or about 4, or about five days, or more than five days, and then a subsequently administered after an appropriate interval for an additional period of time, for example, for about 2, or about 3, or about 4, or about five days, or more than five days. Cycles of treatment may occur in immediate succession or with an interval of no treatment between cycles.

In some embodiments, a composition comprising N1-methyl-pyrazoloanthrone, e.g., N1-methyl 1,9-pyrazoloanthrone as disclosed herein can be administered to a subject before a chemotherapeutic treatment, or radiation treatment is administered to the subject. In alternative embodiments, a composition comprising N1-methyl-pyrazoloanthrone, e.g., N1-methyl 1,9-pyrazoloanthrone as disclosed herein can be co-administered to a subject concurrently with another agent or treatment regimen, e.g., concurrently with a chemotherapeutic treatment, or radiation treatment. In some embodiments, a composition comprising N1-methyl-pyrazoloanthrone, e.g., N1-methyl 1,9-pyrazoloanthrone as disclosed herein can be co-administered with a pharmaceutical composition comprising an comprising one or more addition agents. The pharmaceutical compositions can be provided by pulsed administration. For example, a composition comprising N1-methyl-pyrazoloanthrone, e.g., N1-methyl 1,9-pyrazoloanthrone as disclosed herein can be administered to a subject, followed by a chemotherapeutic treatment, or radiation treatment after an interval of time has passed, and this order of administration the same or similar time interval can be repeated, for example, at least 1, 2, 3, 4, 5, 6, 7, 8, 9, or 10 or more times.

The invention features an article of manufacture that contains packaging material and compounds of the present invention, for example a N1-methyl-pyrazoloanthrone, e.g., a compound of formula (I)-(VII), such as N1-methyl 1,9-pyrazoloanthrone as disclosed herein and/or functional derivatives thereof in a formulation contained within the packaging material. In some embodiments, a formulation can contain at least one of the compounds of the present invention, for example at least one N1-methyl-pyrazoloanthrone compound of formula (I)-(VII), such as N1-methyl 1,9-pyrazoloanthrone as disclosed herein and/or functional derivatives thereof and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference.

More specifically, the invention features an article of manufacture that contains packaging material and at least one of the compounds of the present invention, for example at least one N1-methyl-pyrazoloanthrone compound of formula (I)-(VII), such as N1-methyl 1,9-pyrazoloanthrone as disclosed herein or a functional derivative thereof contained within the packaging material. The packaging material contains a label or package insert indicating that the formulation can be administered to the subject to alleviate a proliferative disorder, for example cancer in an amount, at a frequency, and for a duration effective treat or prevent symptoms associated with such disease states or conditions discussed throughout this specification.

### Pharmaceutical Compositions

In another embodiment of the invention, a pharmaceutical composition can contain one or more compounds as disclosed, e.g., a N1-methyl-pyrazoloanthrone, e.g., a compound of formula (I)-(VII), such as N1-methyl 1,9-pyrazoloanthrone as disclosed herein. For purpose of administration, a N1-methyl-pyrazoloanthrone, e.g., a compound of formula (I)-(VII), such as N1-methyl 1,9-pyrazoloanthrone as disclosed herein is preferably formulated as a pharmaceutical composition. Pharmaceutical compositions of the present invention comprise a compound of this invention and a pharmaceutically acceptable carrier, wherein the compound is present in the composition in an amount which is effective to treat the condition of interest. Preferably, a pharmaceutical composition of the present invention can include a N1-methyl-pyrazoloanthrone, e.g., a compound of formula (I)-(VII), such as N1-methyl 1,9-pyrazoloanthrone as disclosed herein in an amount from 0.1 mg to 250 mg per dosage depending upon the route of administration, and more typically from 1 mg to 60 mg. Appropriate concentrations and dosages can be readily determined by one skilled in the art.

Pharmaceutically acceptable carriers are familiar to those skilled in the art. For compositions formulated as liquid solutions, acceptable carriers include saline and sterile water, and may optionally include antioxidants, buffers, bacteriostats and other common additives. The compositions can also be formulated as pills, capsules, granules, or tablets which contain, in addition to a compound of this invention, diluents, dispersing and surface active agents, binders, and lubricants. One skilled in this art may further formulate the compounds of this invention in an appropriate manner, and in accordance with accepted practices, such as those disclosed in Remington's Pharmaceutical Sciences, Gennaro, Ed., Mack Publishing Co., Easton, Pa. 1990.

While it is possible for compounds of the present invention, for example a N1-methyl-pyrazoloanthrone, e.g., a compound of formula (I)-(VII), such as N1-methyl 1,9-pyrazoloanthrone as disclosed herein and/or functional derivatives thereof, to be administered alone, it is preferable to administer the compound as a pharmaceutical composition.

Formulations of the invention can be prepared by a number or means known to persons skilled in the art. In some embodiments the formulations can be prepared by combining (i) at least a N1-methyl-pyrazoloanthrone, e.g., a compound of formula (I)-(VII), such as N1-methyl 1,9-pyrazoloanthrone as disclosed herein and/or functional derivatives thereof in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

The compositions of the present invention can be in any form. These forms include, but are not limited to, solutions, suspensions, dispersions, ointments (including oral ointments), creams, pastes, gels, powders (including tooth powders), toothpastes, lozenges, salve, chewing gum, mouth sprays, pastilles, sachets, mouthwashes, aerosols, tablets, capsules, transdermal patches, that comprise one or more of the compounds of the present invention, and/or their functional derivatives thereof for oral or subcutaneous administration.

In certain embodiments, the compounds of the present invention, for example a N1-methyl-pyrazoloanthrone, e.g., a compound of formula (I)-(VII), such as N1-methyl 1,9-pyrazoloanthrone as disclosed herein or functional derivatives thereof are administered to a subject as a pharmaceutical composition with a pharmaceutically acceptable carrier. In certain embodiments, these pharmaceutical compositions optionally further comprise one or more additional therapeutic agents. In certain embodiments, the additional therapeutic agent or agents are anti-cancer agents. In some embodiments, the therapeutic agents are chemotherapeutic agents, for example but not limited to, cisplatin, paxicital etc. In some embodiments, the therapeutic agents are radiotherapeutic agents. Examples of chemotherapeutic agents in the pharmaceutical compositions of this invention are, for example but not limited to paclitaxel, cisplatin, doxorubicin and paclitaxel, vermurafib, nitrogen mustards such as cyclophosphamide, ifosfamide, and melphalan; ethylenimines and methylmelamines such as hexamethylmelamine and thiotepa; pyrimidine analogs such as fluorouracil and fluorodeoxyuridine; vinca alkaloids such as vinblastine; epipodophyllotoxins such as etoposide and teniposide; antibiotics such as actinomycin D, doxorubicin, bleomycin, and mithramycin; biological response modifiers such as interferon, platinum coordination complexes such as cisplatin and carboplatin; estrogens such as diethylstilbestrol and ethinyl estradiol; antiandrogens such as flutamine; and gonadotropin releasing hormone analogs such as leuprolide. Other compounds such as decarbazine, nitrosoureas, methotrexate, diticene, and procarbazine are also effective and encompassed for use in the methods of the present invention. Of course, other chemotherapeutic agents which are known to those of ordinary skill in the art can readily be substituted as this list should not be considered exhaustive or limiting.

In some embodiments the pharmaceutical composition comprises compounds of the present invention, for example a N1-methyl-pyrazoloanthrone, e.g., a compound of formula (I)-(VII), such as N1-methyl 1,9-pyrazoloanthrone as disclosed herein and/or functional derivatives thereof, alone or in any plurality of combinations. In other embodiments, the pharmaceutical compositions optionally further comprise one or more additional therapeutic agents including but not limited to paclitaxel, cisplatin, doxorubicin and paclitaxel, vermurafib.

In some embodiments, the pharmaceutical composition comprising a N1-methyl-pyrazoloanthrone, e.g., a compound of formula (I)-(VII), such as N1-methyl 1,9-pyrazoloanthrone as disclosed herein or derivatives thereof as disclosed herein can supplement the treatment of any known additional therapy, including, but not limited to, antibody administration, vaccine administration, administration of cytotoxic agents, natural amino acid polypeptides, nucleic acids, nucleotide analogues, and biologic response modifiers. In some embodiments, additional therapy is, for example, surgery, chemotherapy, radiotherapy, thermotherapy, immunotherapy, hormone therapy and laser therapy. In some embodiments, the additional therapy is chemotherapy. Two or more combined compounds may be used together or sequentially with the pharmaceutical composition comprising a N1-methyl-pyrazoloanthrone, e.g., a compound of formula (I)-(VII), such as N1-methyl 1,9-pyrazoloanthrone as disclosed herein or a derivative thereof. In some embodiments, a N1-methyl-pyrazoloanthrone, e.g., a compound of formula (I)-(VII), such as N1-methyl 1,9-pyrazoloanthrone as disclosed herein or derivatives thereof can be administered before the additional therapy, after the additional therapy or at the same time as the additional therapy. In some embodiments, a N1-methyl-pyrazoloanthrone, e.g., a compound of formula (I)-(VII), such as N1-methyl 1,9-pyrazoloanthrone as disclosed herein or a functional derivative thereof are administered a plurality of times, and in other embodiments, the additional therapies are also administered a plurality of times.

In some embodiments, a N1-methyl-pyrazoloanthrone, e.g., a compound of formula (I)-(VII), such as N1-methyl 1,9-pyrazoloanthrone as disclosed herein or a functional derivative thereof can also be administered in combination with a chemotherapeutic agent (e.g., an anti-cancer agent) as part of an anti-cancer cocktail. An anti-cancer cocktail is a mixture comprising, for example at least one N1-methyl-pyrazoloanthrone, e.g., a compound of formula (I)-(VII), such as N1-methyl 1,9-pyrazoloanthrone as disclosed herein, or functional derivatives thereof, combined with one or more chemotherapeutic agents (or anti-cancer agents) in addition to a pharmaceutically acceptable carrier for delivery. The use of anti-cancer cocktails in the treatment of a subject's cancer is routine. Anti-cancer agents that are well known in the art and can be used as a treatment in combination with a compound of formula (I)-(VII), such as N1-methyl 1,9-pyrazoloanthrone as disclosed herein or functional derivatives thereof, include, but are not limited to: Actinomycin D, Aminoglutethimide, Asparaginase, Bleomycin, Busulfan, Carboplatin, Carmustine, Chlorambucil, Cisplatin (cis-DDP), Cyclophospharnide, Cytarabine HCl (Cytosine arabinoside), Dacarbazine, Dactinomycin, Daunorubicin HCl, Doxorubicin HCl, Estramustine phosphate sodium, Etoposide (V16-213), Flosuridine, S-Fluorouracil (5-Fu), Flutamide, Hydroxyurea (hydroxycarb amide), Ifosfamide, Interferon Alpha-2 a, Interferon Alpha-2b, Leuprolide acetate (LHRH-releasing factor analog), Lomustine, Mechlorethamine HCl (nitrogen mustard), Melphalan, Mercaptopurine, Mesna, Methotrexate (MTX), Mitomycin, Mitoxantrone HCl, Ockeotide, Paclitaxel; Plicamycin, Procarbazine HCl, Streptozocin, Tamoxifen citrate, Thioguanine, Thiotepa, Vinblastine sulfate, Vincristine sulfate, Amsacrine, Azacitidine, Hexamethylmelamine, Interleukin-2, Mitoguazone, Pentostatin, Semustine, Teniposide, and Vindesine sulfate, and analogues thereof. In some embodiments, the anti-cancer agent is selected from the group consisting of paclitaxel, cisplatin, doxorubicin and paclitaxel, vermurafib.

In some embodiments, the methods of the present invention are directed to use of a N1-methyl-pyrazoloanthrone, e.g., a compound of formula (I)-(VII), such as N1-methyl 1,9-pyrazoloanthrone as disclosed herein and functional derivatives thereof with other therapeutic agents, for example chemotherapy agents, wherein the chemotherapy agents, for example paclitaxel, cisplatin, doxorubicin, vermurafib or MIS can be used at a lower dose that results in decreased side effects. For example, in some embodiments the effective dose of the chemotherapeutic agent (e.g., anti-cancer agent) in the presence of a N1-methyl pyrazoloanthrone, e.g., N1-methyl 1,9-pyrazoloanthrone, is lower (e.g., is a decreased unit dose) as compared to the effective dose of the chemotherapeutic agent when used alone or in the absence of the N1-methyl pyrazoloanthrone or functional derivative or analogue thereof. For example, in some embodiments, the effective amount of a chemotherapeutic agent to treat cancer and/or to reduce a tumor size when used in combination with a a compound of formula (I)-(VII), such as N1-methyl 1,9-pyrazoloanthrone is at least 10%, or at least 20% or at least 30% or at least 40%, or at least 50% less (e.g. a decreased dose) than the effective amount of the same chemotherapeutic agent when it is used alone and/or in the absence of N1-methyl-pyrazoloanthrone, e.g., a compound of formula (I)-(VII), such as N1-methyl 1,9-pyrazoloanthrone. Similarly, in some embodiments, the effective amount of a compound of formula (I)-(VII), such as N1-methyl 1,9-pyrazoloanthrone is at least 10%, or at least 20% or at least 30% or at least 40%, or at least 50% less than it's effective amount when used alone and/or in the absence of a chemotherapeutic agent for the treatment of cancer and/or to reduce a tumor size.

In certain embodiments, a pharmaceutical compositions comprising a compound of formula (I)-(VII), such as N1-methyl 1,9-pyrazoloanthrone as disclosed herein or functional derivatives thereof can optionally further comprise one or more additional therapies or agents. In certain embodiments, the additional agent or agents are anti-cancer agents. In some embodiments, the therapeutic agents are chemotherapeutic agents, for example cisplatin, paxicital etc. In some embodiments, the therapeutic agents are radiotherapeutic agents. Examples of chemotherapeutic agents in the pharmaceutical compositions of this invention are, for example nitrogen mustards such as cyclophosphamide, ifosfamide, and melphalan; ethylenimines and methylmelamines such as hexamethylmelamine and thiotepa; pyrimidine analogs such as fluorouracil and fluorodeoxyuridine; vinca alkaloids such as vinblastine; epipodophyllotoxins such as etoposide and teniposide; antibiotics such as actinomycin D, doxorubicin, bleomycin, and mithramycin; biological response modifiers such as interferon, platinum coordination complexes such as cisplatin and carboplatin; estrogens such as diethylstilbestrol and ethinyl estradiol; antiandrogens such as flutamine; and gonadotropin releasing hormone analogs such as leuprolide. Other compounds such as decarbazine, nitrosoureas, methotrexate, diticene, and procarbazine are also effective. Of course, other chemotherapeutic agents which are known to those of ordinary skill in the art can readily be substituted as this list should not be considered exhaustive or limiting.

In some embodiments, a N1-methyl-pyrazoloanthrone, e.g., a compound of formula (I)-(VII), such as N1-methyl 1,9-pyrazoloanthrone as disclosed herein or functional derivatives thereof is administered to a subject with other anti-cancer therapies, for example cancer therapies to which the cancer was previously resistant or refractory.

In some embodiments, the chemotherapeutic agent is MIS, for example recombinant human MIS (rhMIS). In such embodiments, MIS or rhMIS can be prepared and administered, in any form, by any method known by persons of ordinary skill in the art, for example as disclosed in International Patent Application WO92/18152 and European Patent EP584287 and also disclosed in patent Applications WO94/00133 and EP221761, which are incorporated herein in their entity by reference. In some embodiments, the MIS is a modified variant of MIS, as disclosed in U.S. Provisional Application, 61/777,135, filed on Mar. 12, 2013 and 61/880,451, filed Sep. 20, 2013, and PCT application PCT/US2014/024010 which are incorporated herein in their entirety by reference. In some embodiments, a modified variant of MIS comprises amino acids of SEQ ID NO: 1, where the primary cleavage site has been modified to increase cleavage and thus increase the potency and bioactivity of MIS, and the endogenous leader sequence of MIS has been replaced with a different leader sequence (e.g., human serum albumin (HSA) leader sequence) to increase yield of bioactive protein. The HSA leader sequence of SEQ ID NO: 1 can be substituted for a different leader sequence known to persons of ordinary skill in the art, and disclosed in U.S. Provisional Applications 61/777,135, and 61/880,451 and PCT application PCT/US2014/024010. The variant of MIS of SEQ ID NO: 1 is encoded by nucleic acid of SEQ ID NO: 6. In some embodiments, a variant of MIS of SEQ ID NO: 1 is produced by a vector encoding nucleic acid SEQ ID NO: 6, for example a viral vector such as an adenovirus or adeno-associated virus (AAV). In some embodiments, the AAV is AAV9 as disclosed in U.S. Pat. No. 7,906,111, which is incorporated herein in its entirety by reference.

In certain embodiments, the endogenous compounds are isolated and/or purified or substantially purified by one or more purification methods described herein or known by those skilled in the art. Generally, the purities are at least 90%, in particular 95% and often greater than 99%. In certain embodiments, the naturally occurring compound is excluded from the general description of the broader genus.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. The term “pharmaceutically acceptable carriers” is intended to include all solvents, diluents, or other liquid vehicle, dispersion or suspension aids, surface active agents, isotonic agents, thickening or emulsifying agents, preservatives, solid binders, lubricants and the like, as suited to the particular dosage form desired. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its functional derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention.

These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use. These compounds include the zwitterionic forms, where possible, of compounds of the invention.

The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “prodrug” refers to compounds that are rapidly transformed in vivo to yield the compounds of the invention, for example the pyrazoloathrone and functional derivatives thereof of the invention, by hydrolysis in blood. A thorough discussion is provided in T. Higachi and V. Stella, “Pro-drugs as Novel Delivery Systems,” Vol. 14 of the A.C.S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. The prodrug may be designed to alter the metabolic stability or the transport characteristics of a compound, to mask side effects or toxicity, to improve the flavor of a compound or to alter other characteristics or properties of a compound. By virtue of knowledge of pharmacodynamic processes and drug metabolism in vivo, once a pharmaceutically active compound is identified, those of skill in the pharmaceutical art generally can design prodrugs of the compound (see, e.g., Nogrady (1985) Medicinal Chemistry A Biochemical Approach, Oxford University Press, N.Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

In other embodiments of the present invention, a N1-methyl-pyrazoloanthrone, e.g., a compound of formula (I)-(VII), such as N1-methyl 1,9-pyrazoloanthrone as disclosed herein or a functional derivative thereof are conjugated or covalently attached to another targeting agent to increase the specificity of a N1-methyl-pyrazoloathrone and functional derivatives thereof targeting the cell, for example a cancer cell. Targeting agents can include, for example without limitation, antibodies, cytokines and receptor ligands. In some embodiments, the targeting agent is overexpressed on the cells to be targeted, for example the cancer cells as compared to normal cells. In alternative embodiments, the pyrazoloathrone and functional derivatives thereof can be conjugated or covalently attached to compounds that elicit an immune response, such as for example but without limitation, cytokines.

In some embodiments, a N1-methyl-pyrazoloanthrone, e.g., a compound of formula (I)-(VII), such as N1-methyl 1,9-pyrazoloanthrone as disclosed herein or a functional derivative thereof can be conjugated to, by covalent linkage or any other means, to another agent, for example a chemotherapy agent, or recombinant human MIS or functional derivatives and analogues thereof. In some embodiments, a N1-methyl-pyrazoloanthrone, e.g., a compound of formula (I)-(VII), such as N1-methyl 1,9-pyrazoloanthrone as disclosed herein or a functional derivative thereof can be conjugated to a targeting moiety, for example a cancer cell targeting moiety to target the compounds of the present invention to a cancer cell. Such targeting moieties and methods are well known by persons of ordinary skill in the art and are encompassed for use in the methods of the present invention. The conjugation may be a permanent or reversible conjugation.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients. In one aspect, a solution of resolvin and/or protectin or precursor or analog thereof can be administered as eye drops for ocular neovascularization or ear drops to treat otitis.

In some embodiments, the composition used in the methods as described herein can be in a controlled release form. A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

Liquid dosage forms for oral administration of the compounds of the invention include pharmaceutically acceptable emulsions, microemulsions, solutions, suspensions, syrups and elixirs.

In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some instances, pharmaceutical compositions comprising the resolvins and protectins of the invention for the administration of angiogenesis may be in a formulation suitable for rectal or vaginal administration, for example as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore release the active compound. Suitable carriers and formulations for such administration are known in the art.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the compounds (resolvins and/or protectins and/or precursors or analogues thereof) of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

More specifically, the invention features an article of manufacture that contains packaging material and at least one compound of the present invention, for example a N1-methyl-pyrazoloanthrone, e.g., a compound of formula (I)-(VII), such as N1-methyl 1,9-pyrazoloanthrone as disclosed herein or a functional derivative thereof are contained within the packaging material. The packaging material contains a label or package insert indicating that the formulation can be administered to the subject with neovascularization in an amount, at a frequency, and for a duration effective treat or prevent symptoms associated with such disease states or conditions discussed throughout this specification. In some embodiments, the proliferative disorder is a cancer. In some embodiments, the cancer is for example a cancer expressing at least one member of the Casein Kinase I family (e.g., CSNK1A1, CSNK1B, CSNK1G1, CSNK1G2, CSNK1G3, CSNK1D, CSNK1E) and/or ARK5/NUAK1 include, for example, ovarian cancer, cervical cancer, breast cancer, prostate cancer, and endometrial cancer.

Remington's Pharmaceutical sciences Ed. Germany, Merk Publishing, Easton, Pa., 1995 (the contents of which are hereby incorporated by reference), discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its functional derivatives such as sodium carboxymethyl cellulose, ethyl cellulose and cellulose acetate; malt; gelatin; talc; excipients such as cocoa butter and: suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; water; isotonic saline; Ringer's solution, ethyl alcohol, and phosphate buffer solutions, as well as other non-toxic compatible lubricants such as sodium lauryl sulfate and magnesium sulfate, as well as coloring agents, releasing agents, coating agents, sweetening, flavoring and perfuming agents, preservatives and antioxidants can also be present in the composition, according to the judgment of the formulator.

### Uses

In another embodiment, the present invention provides a method for treating a variety of conditions by administering an effective amount of example a N1-methyl-pyrazoloanthrone, e.g., a compound of formula (I)-(VII), such as N1-methyl 1,9-pyrazoloanthrone as disclosed herein or a functional derivative thereof to a subject in need thereof. Conditions that may be treated by the compounds of this invention, or a pharmaceutical composition containing the same, include any condition which is treated or results in the reduction of a symptom by administration of an inhibitor at least one member of the Casein Kinase I family (e.g., CSNK1A1, CSNK1B, CSNK1G1, CSNK1G2, CSNK1G3, CSNK1D, CSNK1E) and/or ARK5/NUAK1, and thereby benefit from administration of a N1-methyl-pyrazoloanthrone, e.g., a compound of formula (I)-(VII), such as N1-methyl 1,9-pyrazoloanthrone as disclosed herein or a functional derivative thereof. Representative conditions in this regard include, for example, but not limited to, cancers that express or overexpress MIS receptors, for example a cancer that expresses at least one member of the Casein Kinase I family (e.g., CSNK1A1, CSNK1B, CSNK1G1, CSNK1G2, CSNK1G3, CSNK1D, CSNK1E) and/or ARK5/NUAK1 include, for example, ovarian cancer, cervical cancer, breast cancer, prostate cancer, and endometrial cancer.

Accordingly, the present invention relates to the use of a N1-methyl-pyrazoloanthrone, e.g., a compound of formula (I)-(VII), such as N1-methyl 1,9-pyrazoloanthrone as disclosed herein or a functional derivative thereof for the treatment of any disorder where administration of an inhibitor of at least one member of the Casein Kinase I family (e.g., CSNK1A1, CSNK1B, CSNKIG1, CSNK1G2, CSNK1G3, CSNK1D, CSNK1E) and/or ARK5/NUAK1 is whole, or part, of the therapeutic regime.

In some embodiments, the methods of the present invention are directed to use of a N1-methyl-pyrazoloanthrone, e.g., a compound of formula (I)-(VII), such as N1-methyl 1,9-pyrazoloanthrone as disclosed herein or a functional derivative thereof with other therapeutic agents, for example chemotherapy agents, wherein the chemotherapy agents, for example, paclitaxel, cisplatin, doxorubicin, vermurafibl or MIS, or MIS variants or modified MIS proteins, such as those disclosed in U.S. provisional patent applications 61/777,135 filed on Mar. 12, 2013, and 61/880,451 filed Sep. 20, 2013, and PCT application PCT/US2014/024010, where the additional therapeutic agents can be used at a lower dose, and thus reducing or decreasing any side effects associated with the chemotherapeutic agents. In some embodiments, a variant of MIS corresponds to SEQ ID NO: 1, where the primary cleavage site has been modified to increase cleavage and thus increase the potency and bioactivity of MIS, and the endogenous leader sequence of MIS has been replaced with a different leader sequence (e.g., human serum albumin (HSA) leader sequence) to increase yield of bioactive protein. The HSA leader sequence of SEQ ID NO: 1 can be substituted for a different leader sequence known to persons of ordinary skill in the art, and disclosed in U.S. Provisional Applications 61/777,135, and 61/880,451 and PCT application PCT/US2014/024010. The variant of MIS comprising amino acids of SEQ ID NO: 1 is encoded by a nucleic acid sequence of SEQ ID NO: 6.

In some embodiments, a compound of formula (I)-(VII), such as N1-methyl 1,9-pyrazoloanthrone as disclosed herein or a functional derivative thereof can be used to protect ovarian reserve and prevent female infertility. In some embodiments, a compound of formula (I)-(VII), such as N1-methyl 1,9-pyrazoloanthrone as disclosed herein or a functional derivative thereof can be used as a reversable contraception in the subject. Where a compound of formula (I)-(VII), such as N1-methyl 1,9-pyrazoloanthrone as disclosed herein or a functional derivative thereof is used in a method to protect ovarian reserve, or as a prophylactic to prevent female infertility or as a reversible contraceptive, a composition comprising a compound of formula (I)-(VII), such as N1-methyl 1,9-pyrazoloanthrone as disclosed herein or a functional derivative thereof is administered to the subject to activate MIS RII equal to or above a particular threshold level, where the threshold level is the minimal level of MIS RII actiation that is needed to achieve a complete block in folliculogenesis in the female subject. It should be noted that the threshold level of MIS RII activation can depend on the subject or the species of the subject. There are a variety of practical situations where controlled or reversible contraception is desired, for example, in veterinary applications. In some embodiments, the female subject is an animal. In some embodiments, the animal is a cat or dog. In some embodiments, the female subject is a human.

It is known in the art that a primordial follicle consists of an oocyte enclosed by a single layer of cells, and oocyte is a female germ cell involved in reproduction. Throughout reproductive life, the total number of primordial follicles (also called the ovarian reserve), steadily declines during the lifetime of the female as a consequence of recruitment and cell death (McGee and Huseh, Endocrine Reviews 2000, 21 200-214). Depletion of the ovarian reserve results in female infertility. Without wishing to be bound by theory, when folliculogenesis is arrested or blocked, primordial follicles are prevented from being recruited, effectively removing one main factor that contributes to the depletion of the ovarian reserve.

Accordingly, a related aspect of the invention relates to use of a compound of formula (I)-(VII), such as N1-methyl 1,9-pyrazoloanthrone as disclosed herein or a functional derivative thereof, in a method of preserving an ovarian reserve in a female subject, comprising administering to the female subject a composition comprising a compound of formula (I)-(VII), such as N1-methyl 1,9-pyrazoloanthrone or a functional derivative thereof. An effective amount or dosage of a composition comprising a compound of formula (I)-(VII), such as N1-methyl 1,9-pyrazoloanthrone as disclosed herein or a functional derivative thereof is administered to reduce the number of primordial follicles being recruited. For example, the number of primordial follicles being recruited is reduced by at least 10%, at least 20%, at least 30%, at least 40%, at least 50%, at least 60%, at least 70%, at least 80%, at least 90%, or at least 99% as compared to when a composition comprising a compound of formula (I)-(VII), such as N1-methyl 1,9-pyrazoloanthrone is not administered. An amount of a composition comprising a compound of formula (I)-(VII), such as N1-methyl 1,9-pyrazoloanthrone administered to the female subject is considered effective when the amount is sufficient to reduce the number of primordial follicles being recruited to a desirable number, or decrease the probability of a primordial being recruited to a desirable value. In some embodiments, the amount of a composition comprising a compound of formula (I)-(VII), such as N1-methyl 1,9-pyrazoloanthrone as disclosed herein that is administered is sufficient to achieve contraception. The desired dose or amount can be administered at one time or divided into subdoses, e.g., via pulsed therapy (such as, e.g., 2-4 subdoses etc) and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. The dosage should not be so large as to cause adverse side effects. The term “normal level” is used herein to refer to the number of primordial follicles being recruited in the absence of a compound of formula (I)-(VII), such as N1-methyl 1,9-pyrazoloanthrone as disclosed herein or a functional derivative thereof.

In some embodiments of the present invention may be defined in any of the following numbered paragraphs:

1. A method for treating a cancer in a subject, the method comprising administering to the subject an effective amount of a pharmaceutical composition comprising a N1-methyl-pyrazoloanthrone.
2. The method of paragraph 1, wherein the N1-methyl-pyrazoloanthrone is a compound of formula (I)-(IV), wherein a compound of formula (I) has the following structure:

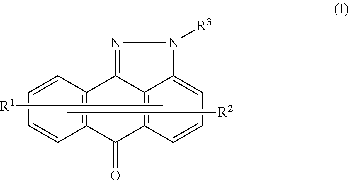

wherein a compound of formula (II):

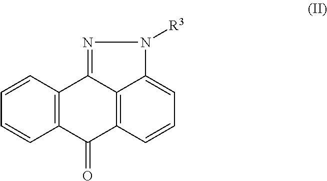

wherein a compound of formula (III) has the following structure:

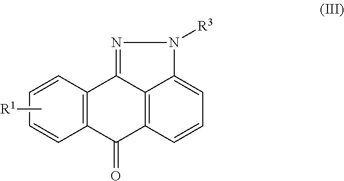

wherein a compound of formula (IV) has the following structure:

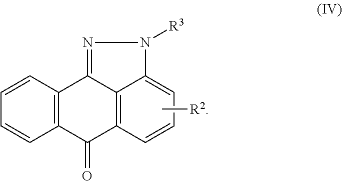

wherein a compound of formula (V) has the following structure:

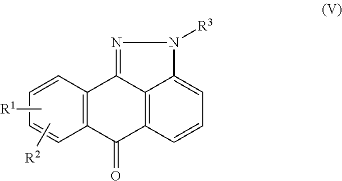

wherein a compound of formula (VI) has the following structure:

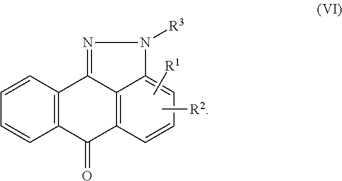

wherein a compound of formula (VII) has the following structure:

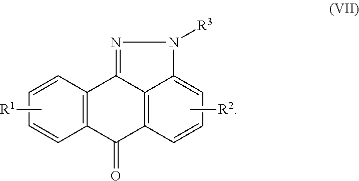

wherein:
R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and
pharmaceutically acceptable salts thereof.
   3. The method of paragraph 2, wherein R³ can be a C₁-C₆ alkyl.
   4. The method of paragraph 2, wherein the a N1-methyl-pyrazoloanthrone is N1-methyl-1,9-pyrazoloanthrone or a functional derivative or a functional analogue thereof.
   5. The method of paragraph 1, wherein the subject is determined to have a cancer expressing at least one member of the Casein Kinase I family or ARK5/NUAK1 kinase.
   6. The method of paragraph 5, wherein the member of the Casein Kinase I family is selected from the group of CSNK1A1, CSNK1B, CSNK1D, CSNK1E, CSNK1G1, CSNK1G2 or CSNK1G3.
   7. The method of paragraph 1, wherein the subject is determined to have at least one genetic alteration in ARK3/NUAK1 gene or at least one genetic alteration in at least one member of the casein kinase 1 family selected from the following genes: CSNK1A1, CSNK1B, CSNK1D, CSNK1E, CSNK1G1, CSNK1G2 or CSNK1G3.
   8. The method of paragraph 1, wherein the subject is determined to have a cancer also expressing a Mullerian Inhibiting Substance (MIS) receptor or MIS Type II receptor (MISRII) or a homologue or functional fragment thereof.
   9. The method of paragraph 1, further comprising administering to the subject an additional chemotherapeutic agent.
   10. The method of paragraph 9, wherein the additional chemotherapeutic agent is selected from cisplatin, doxorubicin, paclitaxel, vemurafinib, MIS, a variant of MIS or a biologically functional homologue thereof.
   11. The method of paragraph 10, wherein a biologically functional homologue of MIS is recombinant human MIS (rhMIS).
   12. The method of paragraph 10, wherein a biologically functional homologue of MIS is a MIS variant and comprises amino acid sequence corresponding to SEQ ID NO: 1.
   13. The method of paragraph 1 or 7, wherein the subject has a cancer selected from the group consisting of: ovarian cancer, prostate cancer, bladder cancer, melanoma, pancreatic cancer, sarcoma, liver cancer, stomach cancer, breast cancer, uterine cancer, adenoid cancer, lung cancer, uterine cancer, colorectal cancer, colon cancer or esophageal cancer.
   14. The method of paragraph 1, wherein the cancer comprises a cancer stem cell.
   15. The method of paragraph 14, wherein the cancer stem cell is an ovarian cancer stem cell, vulvar epidermal cancer stem cell, cervical cancer stem cell, endometrial edenocarinaoma cell and ovarian adenocarcinoma stem cell.
   16. The method of paragraph 1, wherein the cancer is selected from the group consisting of ovarian cancer, vulvar epidermal carcinoma, cervical carcinoma, endometrial edenocarinaoma, ovarian adenocarcinoma, breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, gum cancer, kidney cancer, liver cancer, nasopharynx cancer, prostate cancer, skin cancer, stomach cancer, testis cancer, tongue cancer, or uterine cancer.
   17. The method of paragraph 1, wherein the cancer is a multi-drug resistant cancer.
   18. The method of paragraph 17, wherein the multi-drug resistant cancer is resistant to one or more of: paclitaxel, cisplatin, doxorubicin or vermurafib.
   19. The method of paragraph 1, further comprising administering the composition comprising a N1-methyl-pyrazoloanthrone to the subject if:
a) a biological sample obtained from the subject has increased mRNA or protein expression of at least one member of the Casein Kinase I family or ARK5/NUAK1 kinase; or
b) the subject with cancer is determined to have at one genetic alteration in the ARK3/NUAK1 gene and/or at least one genetic alteration in at least one member of the Casein Kinase 1 family.
   20. The method of paragraph 19, further comprising:
a) measuring the level of protein or mRNA expression of at least one member of the Casein Kinase I family or ARK5/NUAK1 kinase in a biological sample obtained from the subject, or
b) determining if the subject has at least one genetic alteration in ARK3/NUAK1 gene and/or at least one genetic alteration in at least one member of the casein kinase 1 family.
   21. The method of paragraphs 19 or 20, wherein the member of the Casein Kinase I family is selected from the group of CSNK1A1, CSNK1B, CSNK1D, CSNK1E, CSNK1G1, CSNK1G2 or CSNK1G3.
   22. The method of paragraphs 19 or 20, wherein the biological sample is a cancer or tumor tissue sample or a cancer cell or tumor cell or a blood or plasma sample.
   23. The method of paragraphs 19 or 20, wherein the biological sample is a biopsy tissue sample.
   24. A method to reduce the effective dose of a chemotherapeutic agent for the treatment of cancer, the method comprising administering a composition comprising a N1-methyl-pyrazoloanthrone in combination with a composition comprising a chemotherapeutic agent, wherein the effective dose of the chemotherapeutic agent in the presence of the N1-methyl-pyrazoloanthrone is lower as compared to the effective dose of the chemotherapeutic agent in the absence of the N1-methyl-pyrazoloanthrone compound.
   25. The method of paragraph 24, wherein the N1-methyl-pyrazoloanthrone is a compound of formula (I)-(IV), wherein a compound of formula (I) has the following structure:

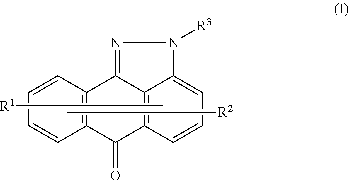

wherein a compound of formula (II):

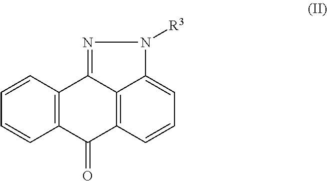

wherein a compound of formula (III) has the following structure:

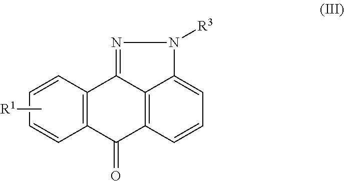

wherein a compound of formula (IV) has the following structure:

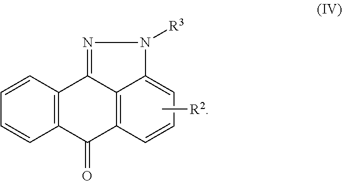

wherein a compound of formula (V) has the following structure:

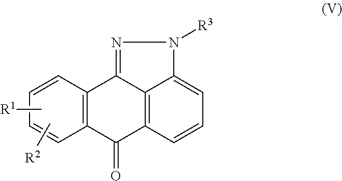

wherein a compound of formula (VI) has the following structure:

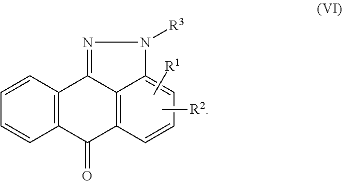

wherein a compound of formula (VII) has the following structure:

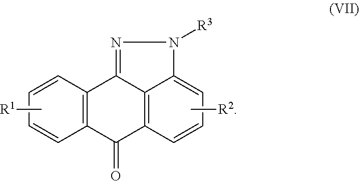

wherein:
R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and
pharmaceutically acceptable salts thereof.
   26. The method of paragraph 25, wherein R³ can be a C₁-C₆ alkyl.
   27. The method of paragraph 25, wherein the N1-methyl-pyrazoloanthrone is N1-methyl-1,9-pyrazoloanthrone or a functional derivative or a functional analogue thereof.
   28. The method of paragraph 24, wherein the cancer expresses at least one member of the Casein Kinase I family or ARK5/NUAK1 kinase.
   29. The method of paragraph 28, wherein the member of the Casein Kinase I family is selected from the group of CSNK1A1, CSNK1B, CSNK1D, CSNK1E, CSNK1G1, CSNK1G2 or CSNK1G3.
   30. The method of paragraph 24, wherein the chemotherapeutic agent is selected from cisplatin, doxorubicin, paclitaxel, rapamycin, vermurafib, MIS or a biologically functional homologue thereof and vemurafinib.
   31. The method of paragraph 30, wherein a biologically functional homologue of MIS is recombinant human MIS (rhMIS).
   32. The method of paragraph 31, wherein a biologically functional homologue of MIS comprises amino acid sequence corresponding to SEQ ID NO: 1.
   33. The method of paragraph 24, wherein the administration of composition comprising N1-methyl-pyrazoloanthrone is concurrently with, prior to, or subsequent to, administration of a composition comprising a chemotherapeutic agent.
   34. The method of paragraph 24, wherein the subject is determined to have at least one genetic alteration in ARK3/NUAK1 gene or at least one genetic alteration in at least one member of the casein kinase 1 family selected from the following genes: CSNK1A1, CSNK1B, CSNK1D, CSNK1E, CSNK1G1, CSNK1G2 or CSNK1G3.
   35. The method of paragraph 24 or 34, wherein the subject has a cancer selected from the group consisting of: ovarian cancer, prostate cancer, bladder cancer, melanoma, pancreatic cancer, sarcoma, liver cancer, stomach cancer, breast cancer, uterine cancer, adenoid cancer, lung cancer, uterine cancer, colorectal cancer, colon cancer or esophageal cancer.
   36. The method of paragraph 24, wherein the cancer comprises an ovarian cancer cell, vulvar epidermal carcinoma cell, cervical carcinoma cell, endometrial edenocarinaoma cell and ovarian adenocarcinoma cell.
   37. The method of paragraph 24, wherein the cancer comprises a cancer stem cell.
   38. The method of paragraph 37, where the cancer stem cell is an ovarian cancer stem cell, vulvar epidermal cancer stem a cell, cervical cancer stem cell, endometrial edenocarinaoma stem cell and ovarian adenocarcinoma stem cell.
   39. The method of paragraph 24, wherein the cancer is selected from the group consisting of ovarian cancer, vulvar epidermal carcinoma, cervical carcinoma, endometrial edenocarinaoma, ovarian adenocarcinoma, breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, gum cancer, kidney cancer, liver cancer, nasopharynx cancer, prostate cancer, skin cancer, stomach cancer, testis cancer, tongue cancer, or uterine cancer.
   40. The method of paragraph 24, wherein the cancer is a multi-drug resistant cancer.
   41. The method of paragraph 40, wherein the multi-drug resistant cancer is resistant to one or more of: paclitaxel, cisplatin, doxorubicin or vermurafib.
   42. The method of paragraph 24, further comprising administering the composition comprising a N1-methyl-pyrazoloanthrone to the subject if:
a) a biological sample obtained from the subject has increased mRNA or protein expression of at least one member of the Casein Kinase I family or ARK5/NUAK1 kinase; or
b) the subject with cancer is determined to have at one genetic alteration in the ARK3/NUAK1 gene and/or at least one genetic alteration in at least one member of the Casein Kinase 1 family.
   43. The method of paragraph 19, further comprising:
a) measuring the level of protein or mRNA expression of at least one member of the Casein Kinase I family or ARK5/NUAK1 kinase in a biological sample obtained from the subject, or
b) determining if the subject has at least one genetic alteration in ARK3/NUAK1 gene and/or at least one genetic alteration in at least one member of the casein kinase 1 family.
   44. The method of paragraphs 42 or 43, wherein the member of the Casein Kinase I family is selected from the group of CSNK1A1, CSNK1B, CSNK1D, CSNK1E, CSNK1G1, CSNK1G2 or CSNK1G3.
   45. The method of paragraphs 42 or 43, wherein the biological sample is a cancer or tumor tissue sample or a cancer cell or tumor cell or a blood or plasma sample.
   46. The method of paragraphs 42 or 43, wherein the biological sample is a biopsy tissue sample.
   47. A pharmaceutical composition comprising a N1-methyl-pyrazoloanthrone, a chemotherapeutic agent and a pharmaceutically acceptable carrier.
   48. The pharmaceutical composition of paragraph 47, wherein the N1-methyl-pyrazoloanthrone is a compound of formula (I)-(IV), wherein a compound of formula (I) has the following structure:

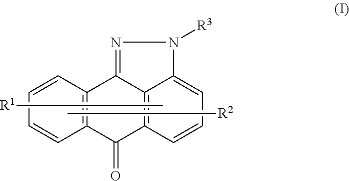

wherein a compound of formula (II):

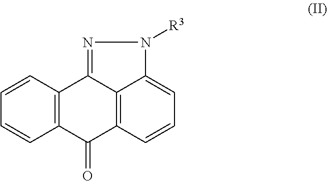

wherein a compound of formula (III) has the following structure:

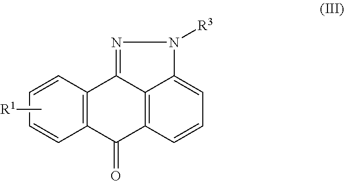

wherein a compound of formula (IV) has the following structure:

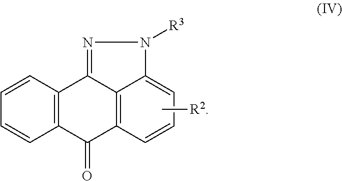

wherein a compound of formula (V) has the following structure:

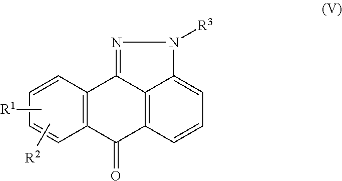

wherein a compound of formula (VI) has the following structure:

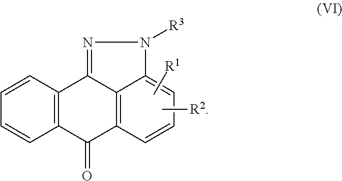

wherein a compound of formula (VII) has the following structure:

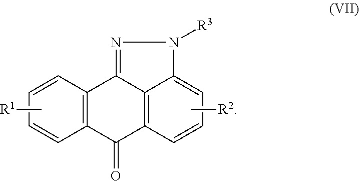

wherein:
R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and
pharmaceutically acceptable salts thereof.
   49. The pharmaceutical composition of paragraph 48, wherein R³ can be a C₁-C₆ alkyl.
   50. The pharmaceutical composition of paragraph 48, wherein the N1-methyl-pyrazoloanthrone is N1-methyl-1,9-pyrazoloanthrone or a functional derivative or a functional analogue thereof.
   51. The pharmaceutical composition of paragraph 47, wherein the chemotherapeutic agent is selected from cisplatin, doxorubicin, paclitaxel, rapamycin, vermurafib, MIS or a biologically functional homologue thereof.
   52. The pharmaceutical composition of paragraph 51, wherein a biologically functional homologue of MIS is recombinant human MIS (rhMIS).
   53. The pharmaceutical composition of paragraph 52, wherein a biologically functional homologue of MIS is a MIS variant which comprises amino acid sequence corresponding to SEQ ID NO: 1.
   54. A method to treat cancer in a subject comprising administering a composition comprising an inhibitor of at least one member of the Casein Kinase I family (e.g., CSNK1A1, CSNK1B, CSNK1G1, CSNK1G2, CSNK1G3, CSNK1D, CSNK1E) and/or ARK5/NUAK1.
   55. The method of paragraph 54, wherein the inhibitor is a N1-methyl-pyrazoloanthrone.
   56. The method of paragraph 55, wherein the N1-methyl-pyrazoloanthrone is a compound of formula (I)-(IV), wherein a compound of formula (I) has the following structure:

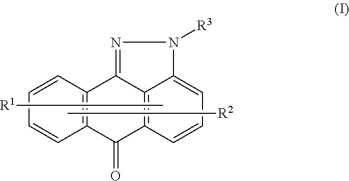

wherein a compound of formula (II):

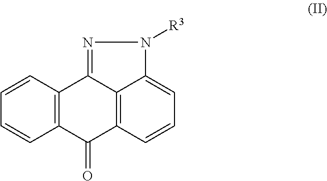

wherein a compound of formula (III) has the following structure:

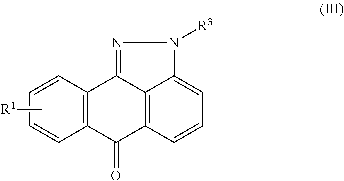

wherein a compound of formula (IV) has the following structure:

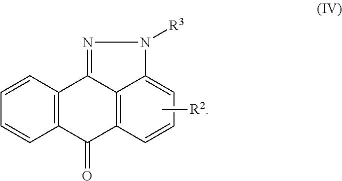

wherein a compound of formula (V) has the following structure:

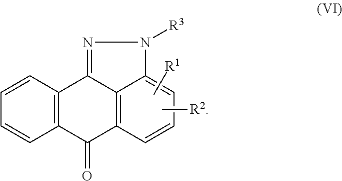

wherein a compound of formula (VI) has the following structure:

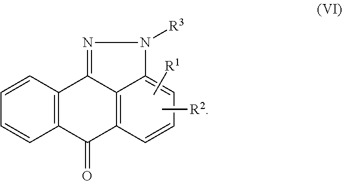

wherein a compound of formula (VII) has the following structure:

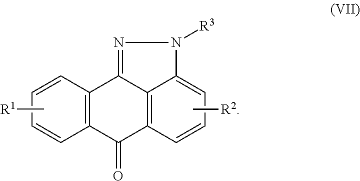

wherein:
R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and
pharmaceutically acceptable salts thereof.
   57. The method of paragraph 56, wherein the N1-methyl-pyrazoloanthrone is N1-methyl-1,9-pyrazoloanthrone or a derivative thereof.
   58. The method of paragraph 54, wherein the subject:
a) has a cancer that expresses at least one member of the Casein Kinase I family or ARK5/NUAK1 kinase;
b) has at least one genetic alteration in ARK3/NUAK1 gene; or
c) has at least one genetic alteration in at least one member of the casein kinase 1 family selected from the following genes: CSNK1A1, CSNK1B, CSNK1D, CSNK1E, CSNK1G1, CSNK1G2 or CSNK1G3.
   59. The method of paragraph 54, further comprising administering the composition to the subject if:
a) a biological sample obtained from the subject has increased mRNA or protein expression of at least one member of the Casein Kinase I family or ARK5/NUAK1 kinase; or
b) the subject with cancer is determined to have at one genetic alteration in the ARK3/NUAK1 gene and/or at least one genetic alteration in at least one member of the Casein Kinase 1 family.
   60. The method of paragraph 59, further comprising:
a) measuring the level of protein or mRNA expression of at least one member of the Casein Kinase I family or ARK5/NUAK1 kinase in a biological sample obtained from the subject, or
b) determining if the subject has at least one genetic alteration in ARK3/NUAK1 gene and/or at least one genetic alteration in at least one member of the casein kinase 1 family.
   61. The method of paragraphs 58 or 59, wherein the member of the Casein Kinase I family is selected from the group of CSNK1A1, CSNK1B, CSNK1D, CSNK1E, CSNK1G1, CSNK1G2 or CSNK1G3.
   62. The method of paragraphs 58 or 59, wherein the biological sample is a cancer or tumor tissue sample or a cancer cell or tumor cell or a blood or plasma sample or saliva sample.
   63. The method of paragraphs 58 or 95, wherein the biological sample is a biopsy tissue sample.
   64. The method of paragraph 59, wherein the cancer also expresses a Mullerian Inhibiting Substance (MIS) receptor, or MISRII.
   65. The method of paragraph 54, further comprising administering to the subject an additional chemotherapeutic agent.
   66. The method of paragraph 65, wherein the additional chemotherapeutic agent is selected from cisplatin, doxorubicin, paclitaxel, vemurafinib, MIS, a variant of MIS or a biologically functional homologue thereof.
   67. Use of a N1-methyl-pyrazoloanthrone for the manufacture of a medicament for treating cancer, wherein the cancer expresses at least one member of the Casein Kinase I family or ARK5/NUAK1 kinase.
   68. The use of paragraph 67, wherein the N1-methyl-pyrazoloanthrone is a compound of formula (I)-(IV), wherein a compound of formula (I) has the following structure:

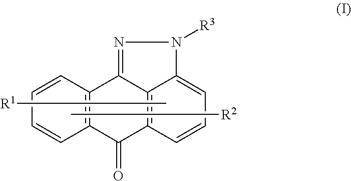

wherein a compound of formula (II):

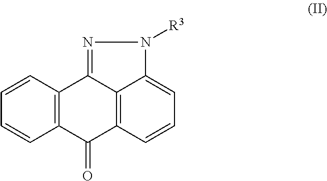

wherein a compound of formula (III) has the following structure:

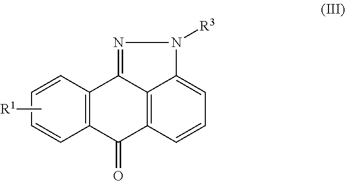

wherein a compound of formula (IV) has the following structure:

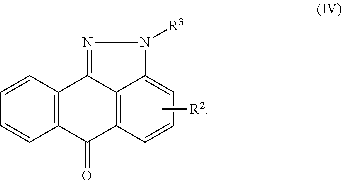

wherein a compound of formula (V) has the following structure:

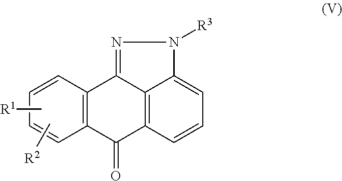

wherein a compound of formula (VI) has the following structure:

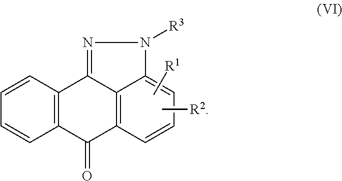

wherein a compound of formula (VII) has the following structure:

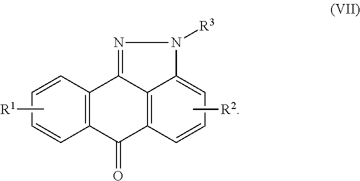

wherein:
R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and
pharmaceutically acceptable salts thereof.
   69. An article of manufacture comprising packaging material and a pharmaceutical composition of paragraph 47, wherein the packaging material comprises a label which indicates the pharmaceutical composition may be administered to a subject for a sufficient term at an effective dose, for treating or reducing the risk of cancer in the subject, where the cancer expresses at least one member of the Casein Kinase I family or ARK5/NUAK1 kinase, or the subject has at least at least one genetic alteration in ARK3/NUAK1 gene and/or at least one genetic alteration in at least one member of the casein kinase 1 family selected from the following genes: CSNK1A1, CSNK1B, CSNK1D, CSNK1E, CSNK1G1, CSNK1G2 or CSNK1G3.
   70. A method of treating a subject with cancer, the method comprising:
a) assessing the expression and/or activity of at least one member of the Casein Kinase I family or ARK5/NUAK1 kinase in a biological sample obtained from the subject, or
b) determining if the subject has at least one genetic alteration in ARK3/NUAK1 gene; or
c) determining if the subject has at least one genetic alteration in at least one member of the casein kinase 1 family selected from the following genes: CSNK1A1, CSNK1B, CSNK1D, CSNK1E, CSNK1G1, CSNK1G2 or CSNK1G3;

wherein a clinician reviews the results and if the results indicate:
d) the presence of increased expression and/or activity of at least one member of the Casein Kinase I family or ARK5/NUAK1 kinase above a predefined threshold level, or
e) the subject has at least one genetic alteration in ARK3/NUAK1 gene; or
f) the subject has at least one genetic alteration in at least one member of the casein kinase 1 family selected from the following genes: CSNK1A1, CSNK1B, CSNK1D, CSNK1E, CSNK1G1, CSNK1G2 or CSNK1G3.

the clinician directs the subject to be treated with pharmaceutical composition of paragraph 47.

71. The method of paragraph 70, wherein the biological sample is selected from the group of; a tissue sample, plasma sample, blood sample, urine sample or saliva sample.
72. The method of paragraph 71, wherein the tissue sample is a cancer or tumor tissue sample or a cancer cell or tumor cell.
73. The method of paragraph 73, wherein the biological sample is a biopsy tissue sample.
74. The method of paragraph 70, wherein the subject has a cancer selected from the group consisting of: ovarian cancer, prostate cancer, bladder cancer, melanoma, pancreatic cancer, sarcoma, liver cancer, stomach cancer, breast cancer, uterine cancer, adenoid cancer, lung cancer, uterine cancer, colorectal cancer, colon cancer or esophageal cancer.
75. The method of paragraph 70, wherein the cancer comprises an ovarian cancer cell, vulvar epidermal carcinoma cell, cervical carcinoma cell, endometrial edenocarinaoma cell and ovarian adenocarcinoma cell.
76. The method of paragraph 70, wherein the cancer comprises a cancer stem cell.
77. The method of paragraph 76, where the cancer stem cell is an ovarian cancer stem cell, vulvar epidermal cancer stem a cell, cervical cancer stem cell, endometrial edenocarinaoma stem cell and ovarian adenocarcinoma stem cell.
78. The method of paragraph 70, wherein the cancer is selected from the group consisting of ovarian cancer, vulvar epidermal carcinoma, cervical carcinoma, endometrial edenocarinaoma, ovarian adenocarcinoma, breast cancer, lung cancer, head and neck cancer, bladder cancer, stomach cancer, cancer of the nervous system, bone cancer, bone marrow cancer, brain cancer, colon cancer, esophageal cancer, endometrial cancer, gastrointestinal cancer, gum cancer, kidney cancer, liver cancer, nasopharynx cancer, prostate cancer, skin cancer, stomach cancer, testis cancer, tongue cancer, or uterine cancer.
79. The method of paragraph 70, wherein if the clinician directs the subject to be treated with pharmaceutical composition of paragraph 47, the method further comprises administering the subject an additional chemotherapeutic agent.
80. The method of paragraph 79, wherein the additional chemotherapeutic agent is selected from cisplatin, doxorubicin, paclitaxel, vemurafinib, MIS, a variant of MIS or a biologically functional homologue thereof.
81. The method of paragraph 80, wherein a biologically functional homologue of MIS is recombinant human MIS (rhMIS).
82. The method of paragraph 81, wherein a biologically functional homologue of MIS comprises amino acid sequence corresponding to SEQ ID NO: 1.

### EXAMPLES

The examples presented herein relate to the use of N1-methyl-1,9-pyrazoloathrone and functional derivatives thereof for the treatment of cancers using a JNK-independent mechanism. N1-methyl-1,9-pyrazoloathrone (M-SP600) and functional derivatives thereof can also be used in conjunction with other therapeutic agents, for example cisplatin, doxorubicin, paclitaxel and vemurafinib to reduce their therapeutic effective dose in the treatment of cancers. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Example 1

### Introduction of a Methyl Group at the N1 Position of Pyrazoloanthrone Abrogates JNK Antagonist and MISR2 Agonist Activities

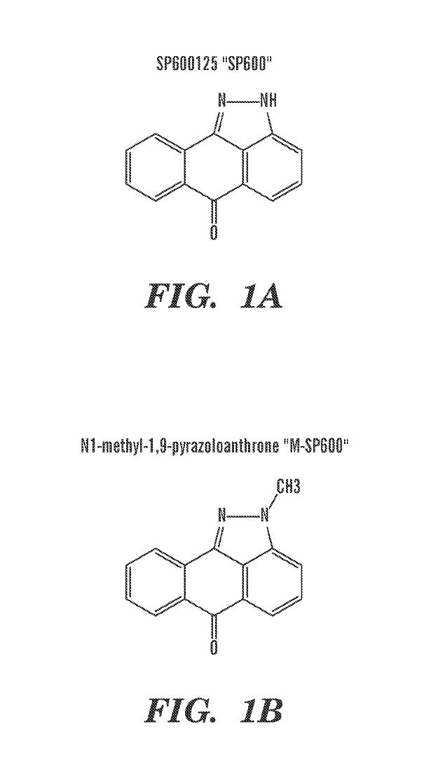

To determine if the methyl modification to the SP600 structure which generates M-SP600 (FIG. 1) impacts the activity profile of the drug, the inventors tested micromolar concentrations in gold standard assays of JNK and MIS activity.

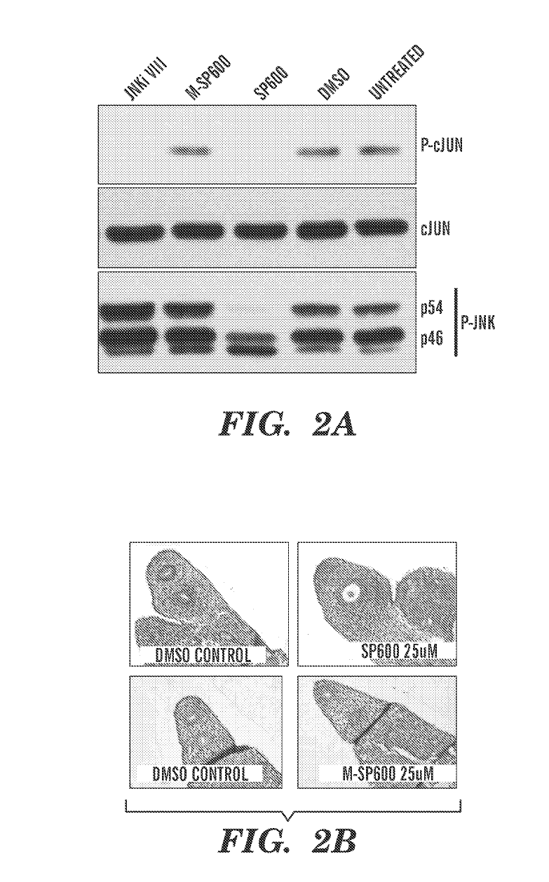

The ptD primary ovarian cancer cell line derived from ascites was grown in a 6 well plate and treated for 30 min with either JNK inhibitors (SP600, JNK inhibitor VIII), M-SP600, vehicle control (DMSO) or were left untreated. Protein lysates were analyzed by western blot and probed with c-JUN and p-c-JUN antibodies, a canonical downstream target of JNKs. c-JUN phosphorylation was inhibited by SP600 (25 uM), JNK inhibitor VIII (25 uM) but not M-SP600 (25 uM) (FIG. 2a). To test if SP600 or M-SP600 cross-react with other kinases of the MAP-kinase family, the inventors also probed the western with a p-JNK antibody. SP600 but not M-SP600 and JNK inhibitor type VIII has off-target effects and inhibits the phosphorylation of the P54 and P46 JNK isoforms (FIG. 2A).

To determine if SP600 and M-SP600 have MISR2 (MISRII) agonist activity, the compounds were tested in the rat fetal urogenital ridge (UGR) assay. UGR tissue is dissected from 14.5d female fetal rats and cultured ex-vivo on a semi solid substrate (agarose droplet on a metal grid) suspended over media at the air/media interface. The UGR from each female is cultured for 72 h in presence of the drugs while the contralateral ridge is cultured in vehicle (DMSO) as a control. The regression of the Mullerian duct, which is specific to MIS activity, is measured in sections of UGR after fixing and embedding. SP600 but not M-SP600 induces regression of the Mullerian duct at 25 uM (FIG. 2B). Surprisingly, the inventors discovered that the treatment with M-SP600 results in smaller Wolffian ducts.

### Example 2

### SP600 and M-SP600 Inhibit Cancer Cell Growth with Similar Dose-Responses

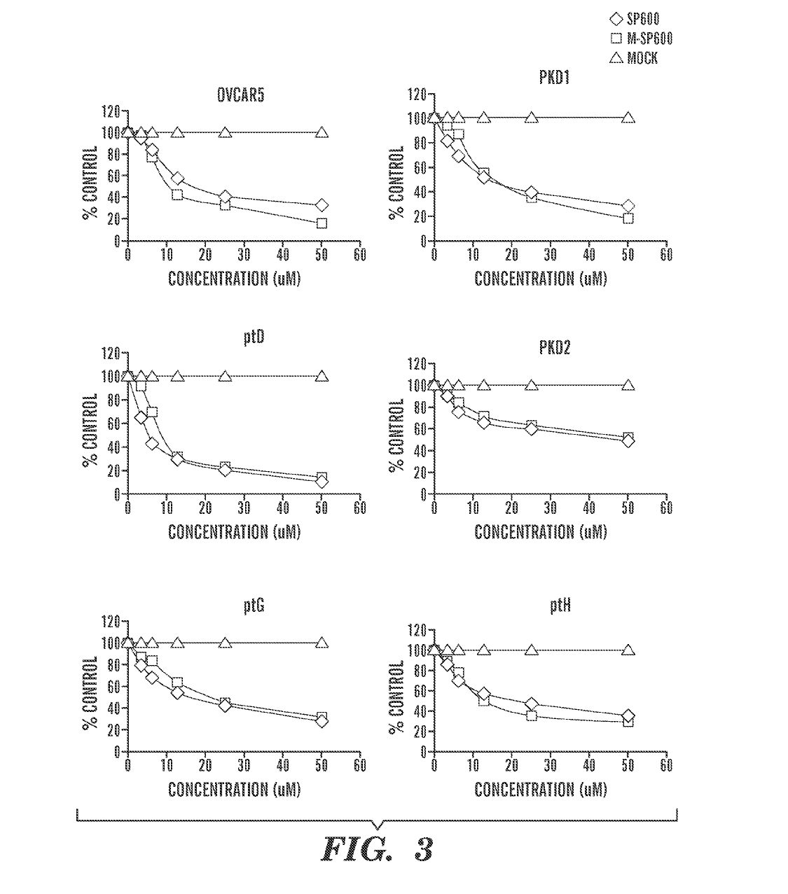

To determine if the SP600 and M-SP600 molecules inhibit cancer cell growth, dose responses to serial dilutions of the drugs were tested in MTT in 96 well plates. Cells were treated for 72 h with SP600, M-SP600, and DMSO vehicle control (Mock). Drug responses were evaluated in the well-established OVCAR5 cancer cell line and a variety of primary ovarian cancer cells lines derived from patient ascites (ptD, ptH, ptG, PKD1, PKD2). All cell lines were inhibited by both SP600 and M-SP600 with remarkably similar dose-response profiles (FIG. 3). The most sensitive cell lines were ptD (IC50—6.25 uM) and OVCAR5 (IC50—12.5 uM).

### Example 3

### Combination Treatments of M-SP600 with Other Chemotherapeutics or Targeted Therapies Significantly Potentiate Cancer Cell Inhibition

To examine if M-SP600 enhances the anticancer efficacy of other chemotherapeutic used in ovarian cancer, combination treatments of M-SP600 and cisplatin (CIS) or doxorubicin (DOX) were tested at a range of doses in MTT during 72 h of treatment in both OVCAR5 and ptD. Combinations of M-SP600 with DOX or with CIS were significantly more inhibitory than either treatment alone in both OVCAR5 and ptD (data not shown).

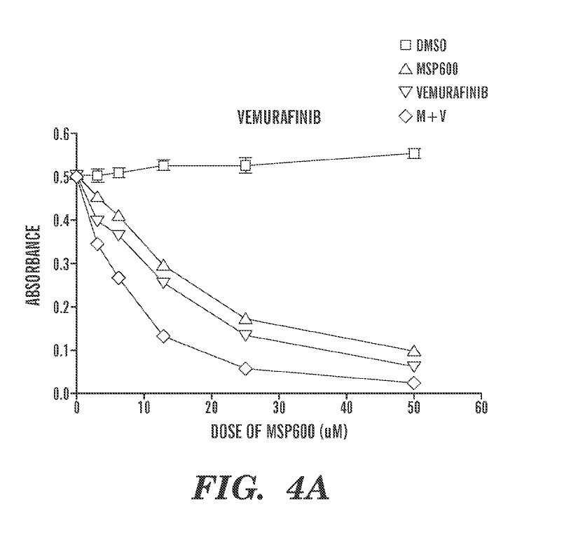

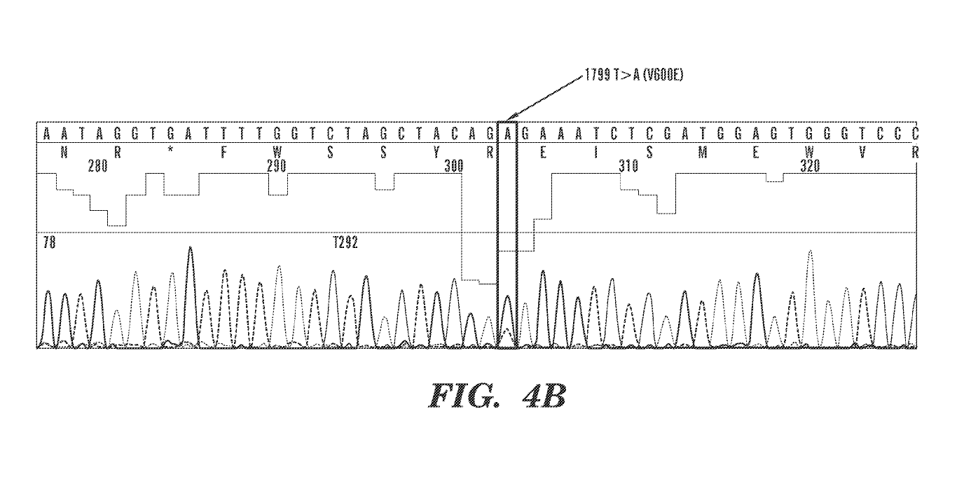

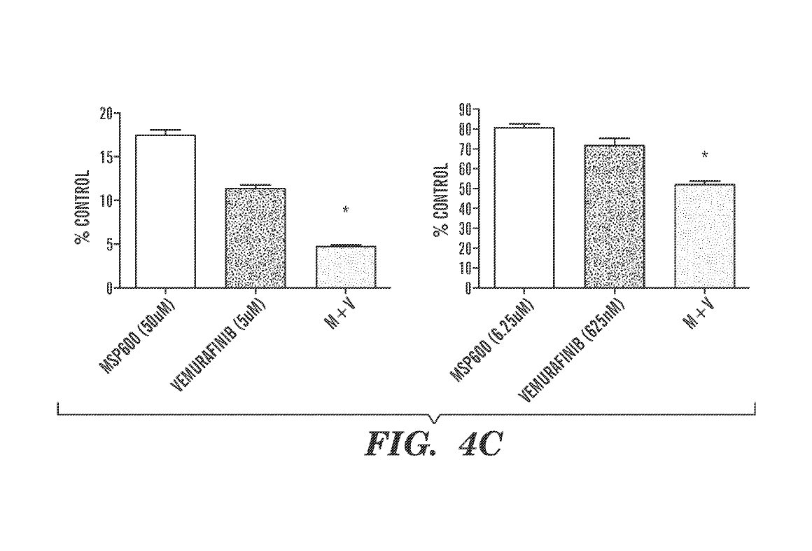

Similarly drug combinations of M-SP600 and Vemurafinib were tested in ptD since the original patient's tumor and the cell line derived from the ascites contain a BRAF V600E mutation. ptD cells were exquisitely sensitive to the targeted BRAF inhibitor Vemurafinib and the combination of M-SP600 and Vemurafinib was significantly better then either treatments alone in a super additive way (FIG. 4).

### Example 4

### Treatment with SP600 or M-SP600 Differentially Alter Cell Cycle Distribution, and Upregulates CDKi P21 and P15

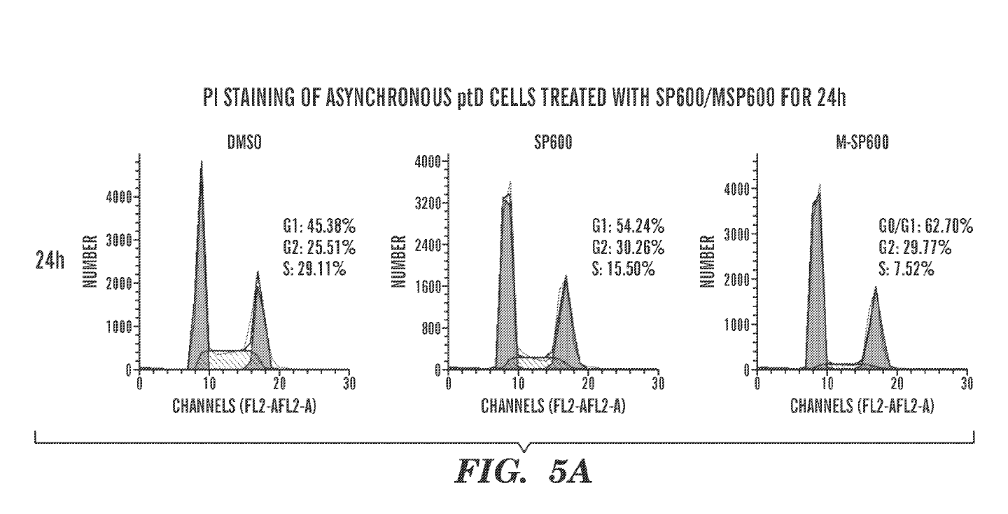

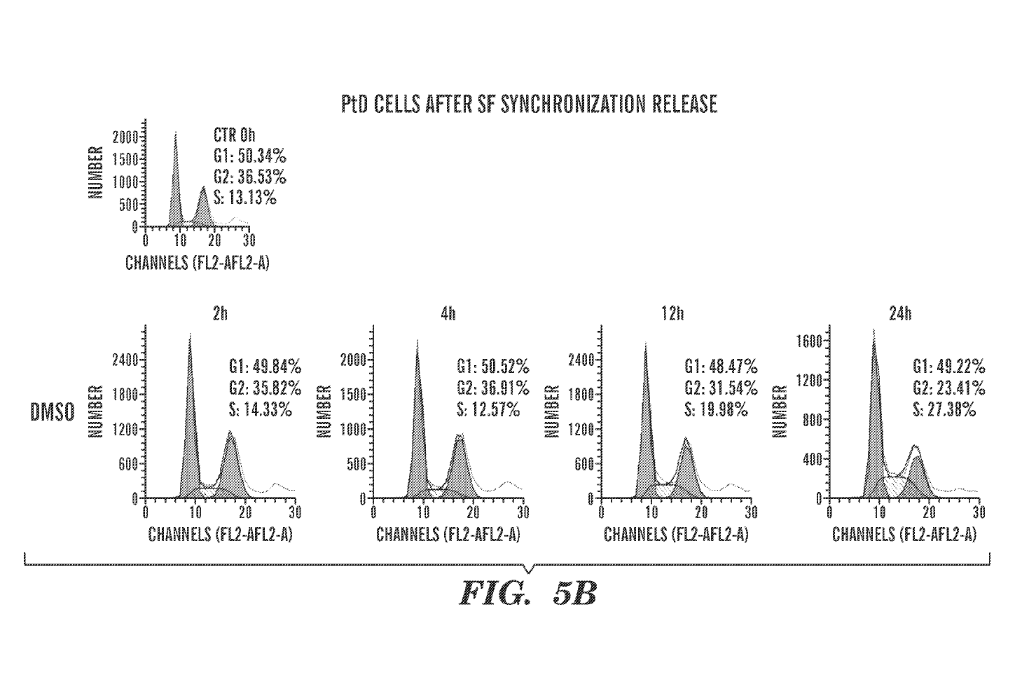

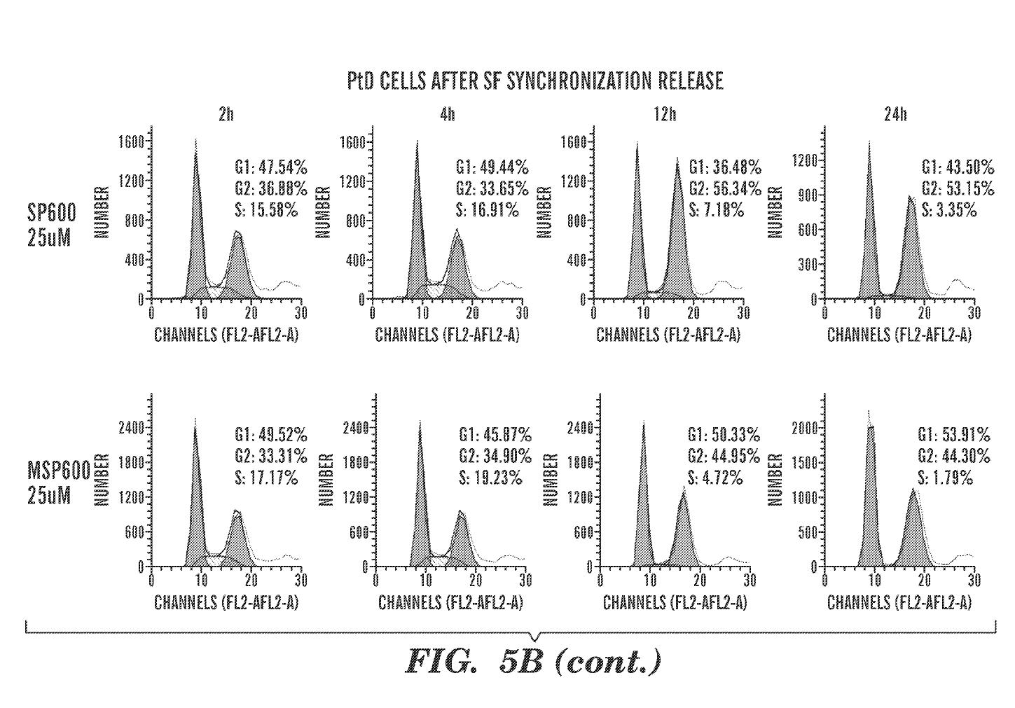

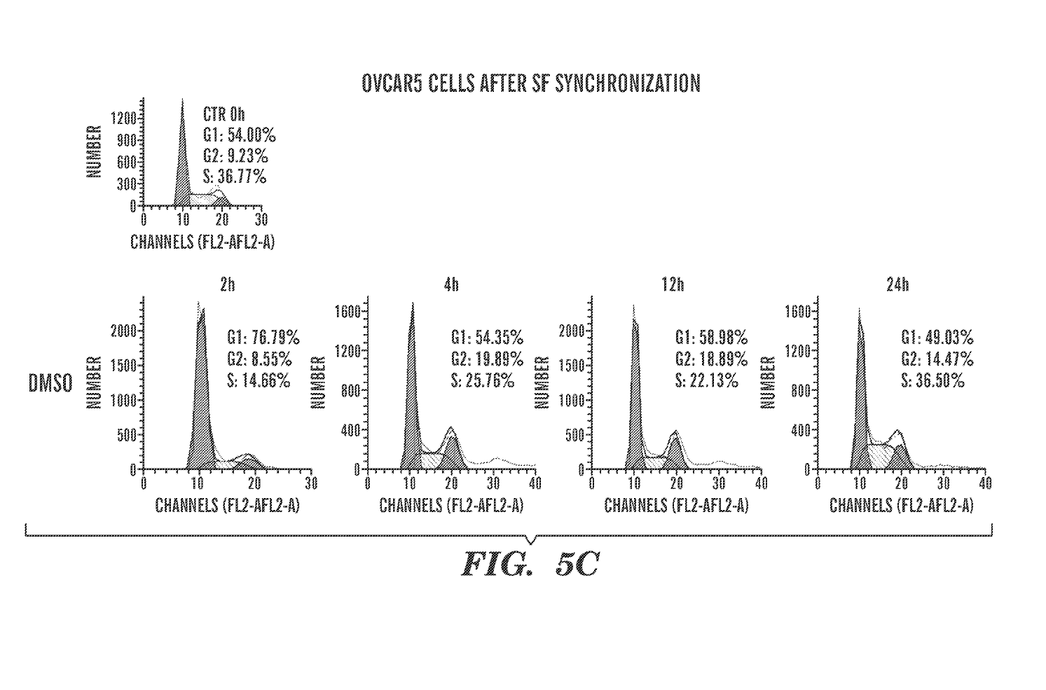

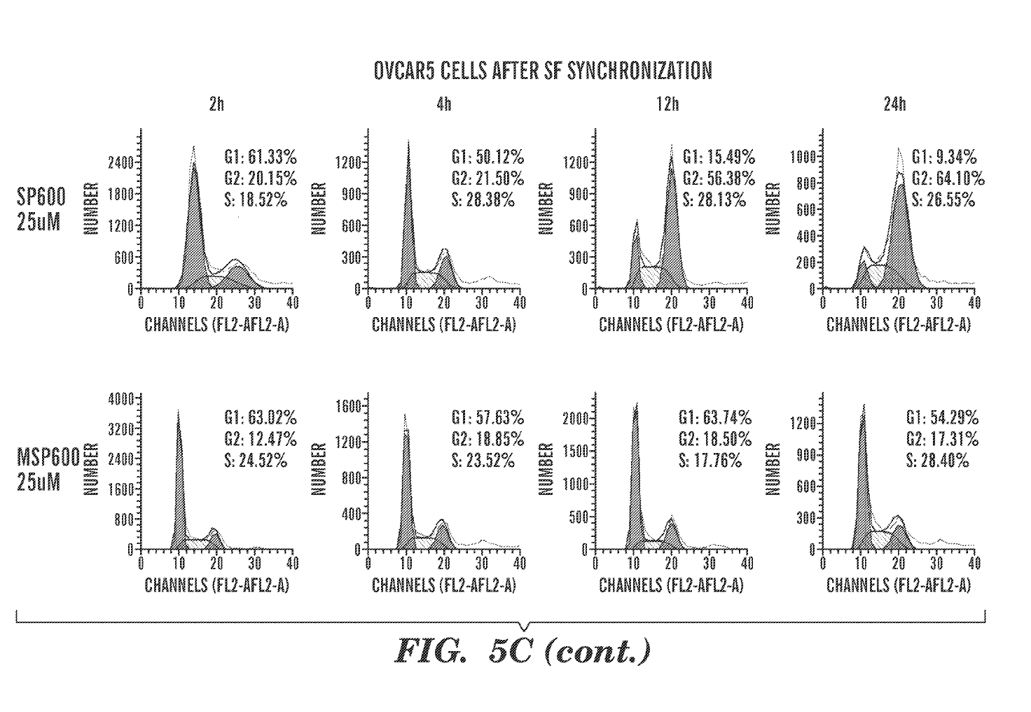

Asynchronous ptD cells treated with either SP600 (25 uM), M-SP600 (25 uM) or DMSO control for 24 h resulted in a marked reduction in cells in S phase accompanied by an accumulation of cells particularly in G1 and less so in G2. The effect was more pronounced with M-SP600 than SP600 (FIG. 5A).

To better define the timing and nature of the block both ptD and OVCAR5 cells were synchronized by serum deprivation for 24 h and then treated with SP600 (25 uM), M-SP600 (25 uM) or DMSO in the presence of 10% serum. While cells from ptD and OVCAR5 initially enter S-phase at the same rate at the 2-4 h time points in the presence of SP600, M-SP600 or DMSO, those cells seem to accumulate and remain blocked in G2 by 12 h while the remaining cells accumulate in G1 and the S-phase fraction all but disappears in the treated groups (FIG. 5B ND 5C). While the inventors detected both G1 and G2 accumulation, SP600 seems to preferentially lead to accumulation of cells in G2 whereas M-SP600 causes a G1 block (FIGS. 5B and 5C). This pattern is observed in both cells lines, but is particularly pronounced in OVCAR5 (FIG. 5C).

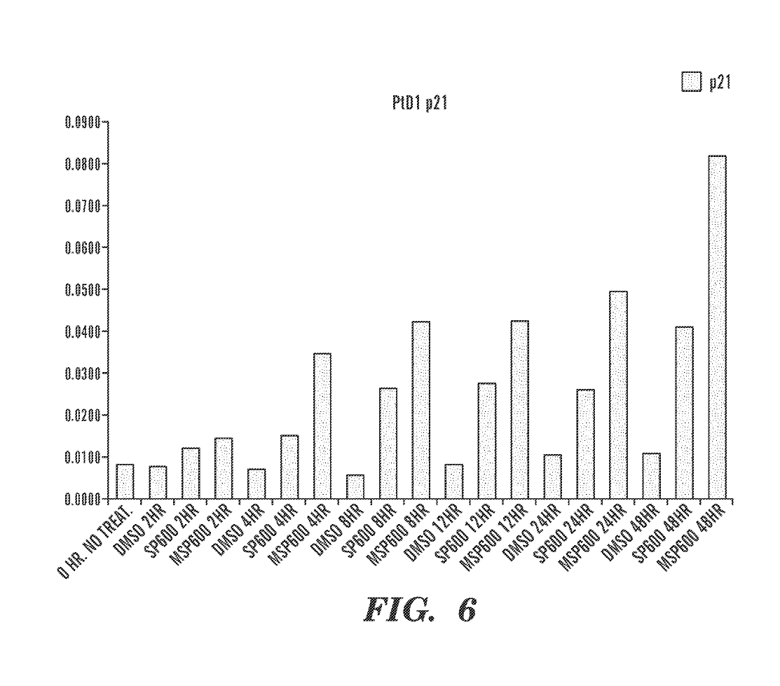

To decipher the mechanism of cell cycle block observed by treatment with M-SP600, the inventors next assessed the expression of the kinases CDKi P15 and P21 by qPCR, since these kinases control G1/S transition. Interestingly, while both SP600 and M-SP600 induce P21 in ptD cells at all times points (2 h, 4 h, 8 h, 12, 24 h and 48 h), it is especially pronounced at 48 h and is greater for the M-SP600 than the SP600 (FIG. 6).

### Example 5

### M-SP600 Inhibits a Small Subset of the Kinases Inhibited by SP600

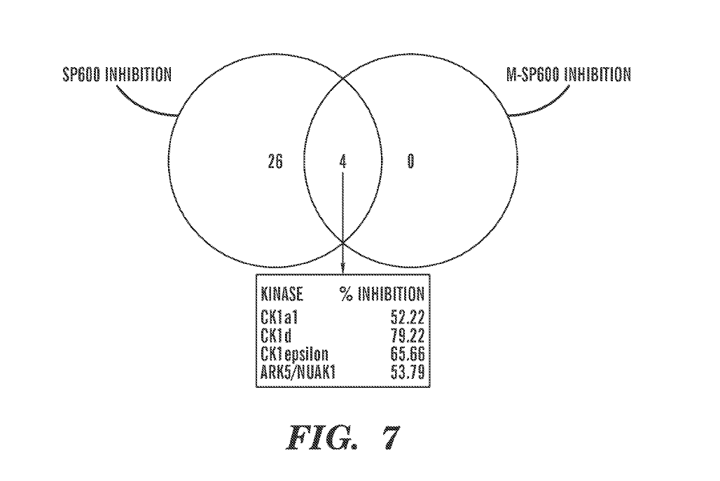

Analysis of the kinase inhibition patterns of SP600 and M-SP600 from the Kinase Inhibitor Resource at Fox Chase Cancer Center, which screened 300 kinases individually in the presence of 178 kinase inhibitors revealed different patterns of inhibition between SP600 and M-SP600. When looking at targets inhibited at more than 50% in presence of 0.5 μM of inhibitor and 10 uM of ATP, SP600 inhibited over 31 different kinases, including JNK2, whereas M-SP600 only inhibited 4, all of which were also inhibited by SP600 (FIG. 7). Those kinases included three members of the casein kinase family (CK1a1, CK1d, CK1e), and ARK5/NUAK1.

### Example 6

### Both SP600 and M-SP600 Inhibit Tumor Growth in a Xenograft Model of Ovarian Cancer

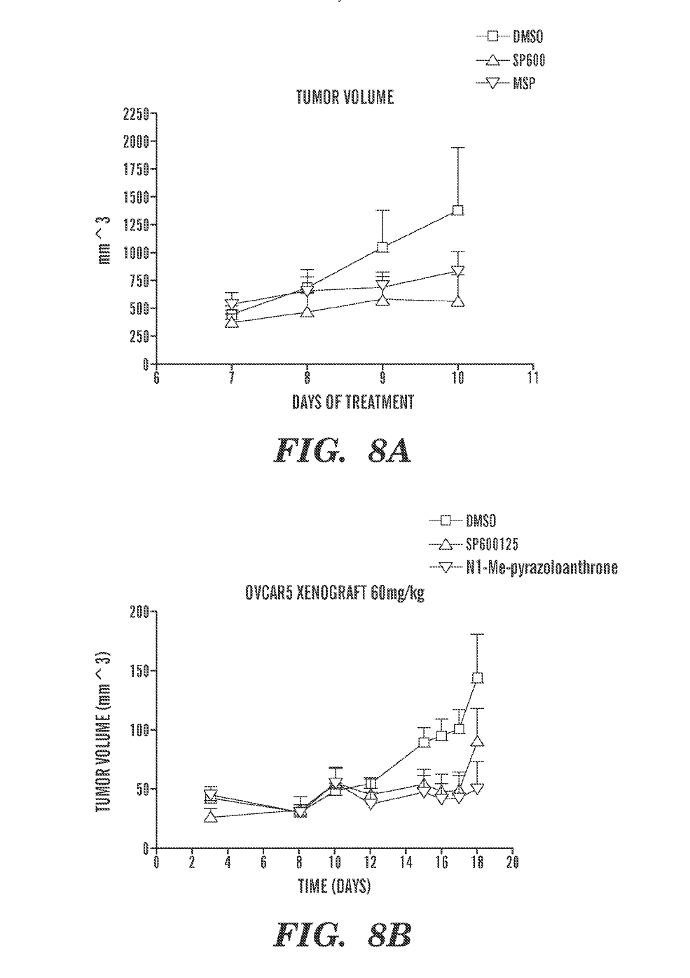

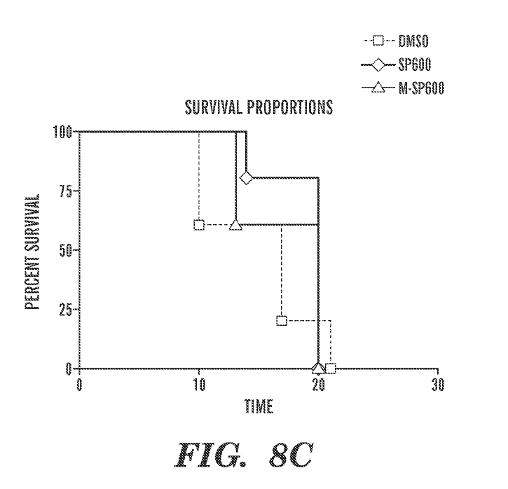

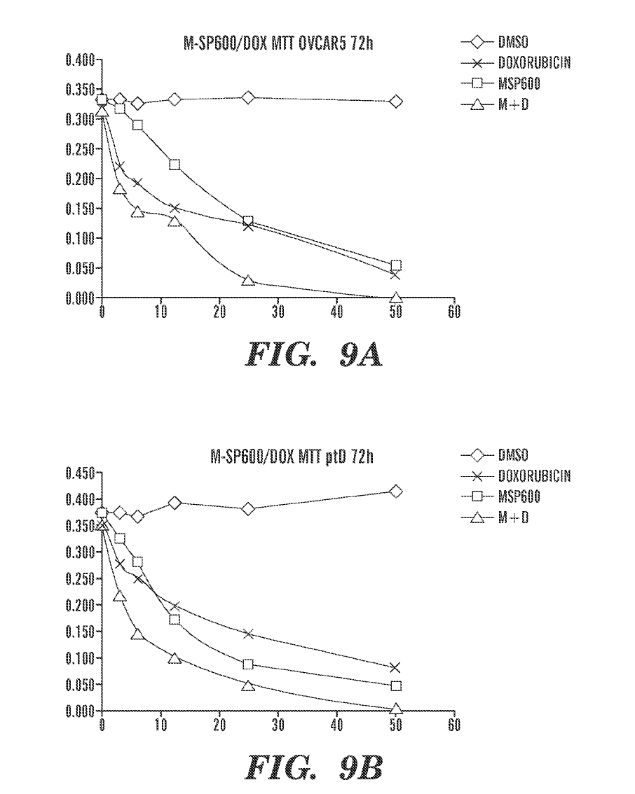

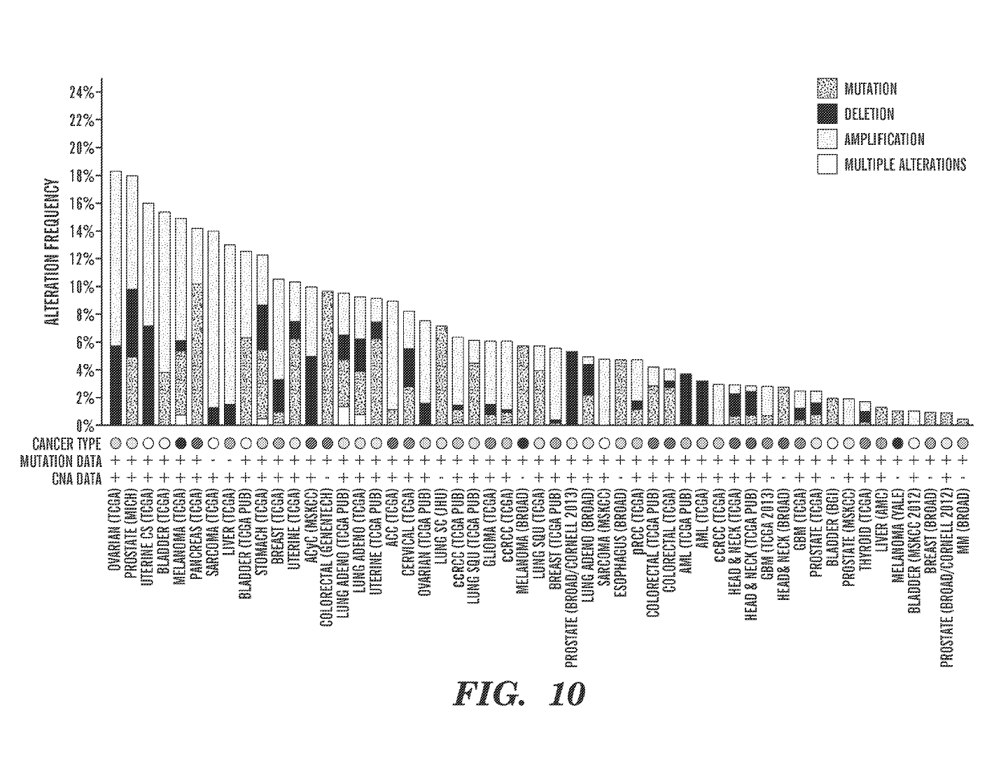

Both SP600 and M-SP600 were first evaluated in PtD cells xenografted at 1×10⁶ in the flank of nude mice and one week later treated for 3 consecutive days at 30 mg/kg/day or with vehicle control (DMSO) in 3 groups (N=5) of randomly selected mice. Both Drugs were well tolerated and a strong trend emerged of inhibition of tumor growth (FIG. 8A).

A longer experiment was subsequently carried out using OVCAR5 cells xenografted at 1×10⁶ in the flank of nude mice, which were treated 72 h later in a 5 days on 2 days off schedule for 3 weeks at a dose of 60 mg/kg/day using 3 groups of 5 mice randomly selected. Tumor growth was significantly inhibited in both the SP600 and the M-SP600 when compared to control at day 15 (FIG. 8B).

Accordingly, the inventors demonstrate herein that M-SP600 is an effective anti-cancer agent in vitro and in vivo, inhibiting the growth of ovarian cancer cells in vivo, and promoting cell cycle arrest of cancer cells in vitro. Moreover, the inventors surprisingly demonstrate that unlike SP600, M-SP600 does not inhibit JNK, and thus M-SP600 functions to inhibit cancer by a JNK-independent mechanism. Furthermore, the inventors demonstrate that surprisingly, unlike SP600, M-SP600 does no activate MISRII. The inventors also demonstrate that unlike SP600, M-SP600 inhibits significantly fewer (only 4) kinases from the Kinase Inhibitor Resource center as compared to 34 kinases by SP600, demonstrating a significantly improved toxicity profile with fewer off-target side effects. Moreover, despite not activating MISRII, the inventors have also discovered that M-SP600 also is highly synergistic with other chemotherapeutic agents, such as, for example, cisplatin, doxorubicin, paclitaxel, and vermurafinib, thus enabling a reduced therapeutic dose of these compounds to be used. Finally, the inventors demonstrate that M-SP600 alone, or in combination with MIS, inhibit ovarian cancer stem cells.

### REFERENCES

The references cited herein and throughout the application are incorporated herein in their entirety by reference.

1. A method for treating a cancer in a subject, the method comprising administering to the subject an effective amount of a pharmaceutical composition comprising a N1-methyl-pyrazoloanthrone.

2. The method of claim 1, wherein the N1-methyl-pyrazoloanthrone is a compound of formula (I)-(IV), wherein a compound of formula (I) has the following structure: 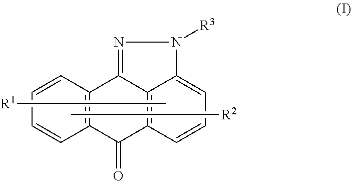
wherein a compound of formula (II): 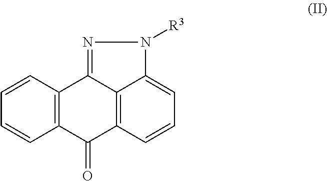
wherein a compound of formula (III) has the following structure: 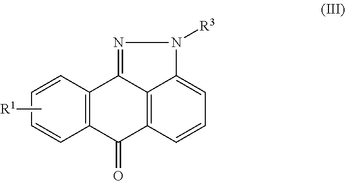
wherein a compound of formula (IV) has the following structure: 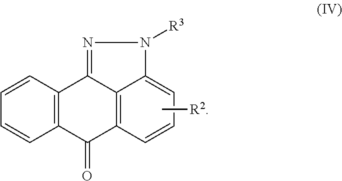
wherein a compound of formula (V) has the following structure: 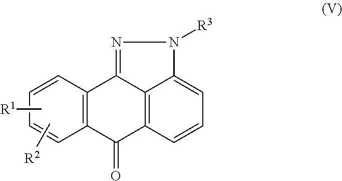
wherein a compound of formula (VI) has the following structure: 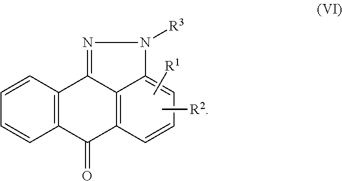
wherein a compound of formula (VII) has the following structure: 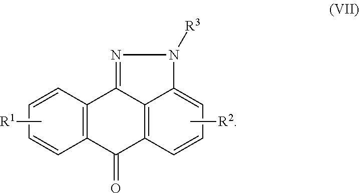
wherein:
R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and
pharmaceutically acceptable salts thereof.

3. The method of claim 2, wherein R³ can be a C₁-C₆ alkyl.

4. The method of claim 2, wherein the a N1-methyl-pyrazoloanthrone is N1-methyl-1,9-pyrazoloanthrone or a functional derivative or a functional analogue thereof.

5. The method of claim 1, wherein the subject is determined to have at least one of:
(i) a cancer expressing at least one member of the Casein Kinase I family or ARK5/NUAK1 kinase; or
(ii) a genetic alteration in ARK3/NUAK1 gene; or
(iii) a genetic alteration in at least one member of the casein kinase 1 family selected from the following genes: CSNK1A1, CSNK1B, CSNK1D, CSNK1E, CSNK1G1, CSNK1G2 or CSNK1G3.

6. (canceled)

7. (canceled)

8. The method of claim 1, wherein the subject is determined to have a cancer also expressing a Mullerian Inhibiting Substance (MIS) receptor or MIS Type II receptor (MISRII) or a homologue or functional fragment thereof.

9. (canceled)

10. The method of claim 1, further comprising administering to the subject an additional chemotherapeutic agent selected from the group consisting of: cisplatin, doxorubicin, paclitaxel, vemurafinib, Mullerian Inhibiting Substance (MIS), recombinant human MIS (rhMIS) or a variant of MIS or a biologically functional homologue thereof.

11.-15. (canceled)

16. The method of claim 1, wherein the cancer is selected from the group consisting of ovarian cancer, vulvar epidermal carcinoma, cervical carcinoma, endometrial edenocarinaoma, ovarian adenocarcinoma, breast cancer, lung cancer, head and neck cancer, bladder cancer, cancer of the nervous system, bone cancer, bone marrow cancer, brain cancer, colon cancer, colorectal cancer, esophageal cancer, endometrial cancer, gastrointestinal cancer, gum cancer, kidney cancer, liver cancer, nasopharynx cancer, prostate cancer, skin cancer, stomach cancer, testis cancer, tongue cancer, melanoma, pancreatic cancer, sarcoma, adenoid cancer or uterine cancer.

17. (canceled)

18. The method of claim 1, wherein the cancer is resistant to one or more of: paclitaxel, cisplatin, doxorubicin or vermurafib.

19. The method of claim 1, further comprising administering the composition comprising a N1-methyl-pyrazoloanthrone to the subject if:
a. a biological sample obtained from the subject has increased mRNA or protein expression of at least one member of the Casein Kinase I family or ARK5/NUAK1 kinase; or
b. the subject with cancer is determined to have at one genetic alteration in the ARK3/NUAK1 gene and/or at least one genetic mutation in at least one member of the Casein Kinase 1 family.

20.-23. (canceled)

24. A method to reduce the effective dose of a chemotherapeutic agent for the treatment of cancer, the method comprising administering to a subject a composition comprising a N1-methyl-pyrazoloanthrone in combination with a composition comprising a chemotherapeutic agent, wherein the effective dose of the chemotherapeutic agent in the presence of the N1-methyl-pyrazoloanthrone is lower as compared to the effective dose of the chemotherapeutic agent in the absence of the N1-methyl-pyrazoloanthrone compound.

25. The method of claim 24, wherein the N1-methyl-pyrazoloanthrone is a compound of formula (I)-(IV), wherein a compound of formula (I) has the following structure: 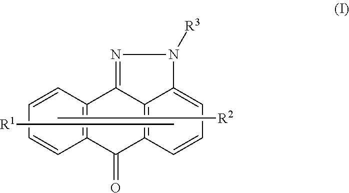
wherein a compound of formula (II): 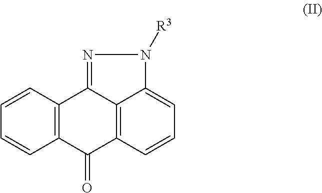
wherein a compound of formula (III) has the following structure: 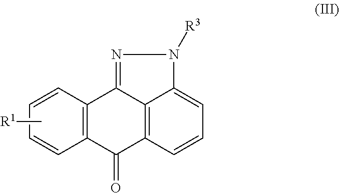
wherein a compound of formula (IV) has the following structure: 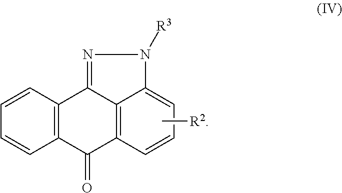
wherein a compound of formula (V) has the following structure: 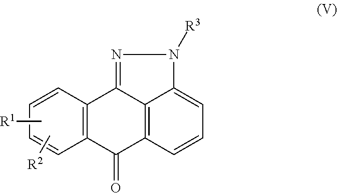
wherein a compound of formula (VI) has the following structure: 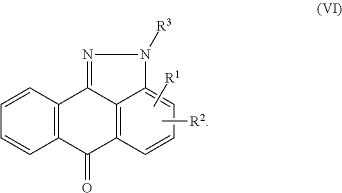
wherein a compound of formula (VII) has the following structure: 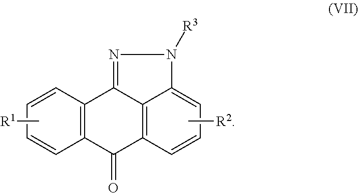
wherein:
R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR⁵), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and
pharmaceutically acceptable salts thereof.

26. The method of claim 25, wherein R³ can be a C₁-C₆ alkyl.

27. The method of claim 25, wherein the N1-methyl-pyrazoloanthrone is N1-methyl-1,9-pyrazoloanthrone or a functional derivative or a functional analogue thereof.

28. The method of claim 24, wherein the subject is determined to have at least one of:
(i) a cancer expresses at least one member of the Casein Kinase I family or ARK5/NUAK1 kinase;
(ii) a genetic alteration in ARK3/NUAK1 gene; or
(iii) a genetic alteration in at least one member of the casein kinase 1 family selected from the following genes: CSNK1A1, CSNK1B, CSNK1D, CSNK1E, CSNK1G1, CSNK1G2 or CSNK1G3.

29. (canceled)

30. The method of claim 24, wherein the chemotherapeutic agent is selected from cisplatin, doxorubicin, paclitaxel, rapamycin, vermurafib, Mullerian Inhibiting Substance (MIS), recombinant human MIS (rhMIS) or a biologically functional homologue thereof.

31.-38. (canceled)

39. The method of claim 24, wherein the cancer is selected from the group consisting of ovarian cancer, vulvar epidermal carcinoma, cervical carcinoma, endometrial edenocarinaoma, ovarian adenocarcinoma, breast cancer, lung cancer, head and neck cancer, bladder cancer, cancer of the nervous system, bone cancer, bone marrow cancer, brain cancer, colon cancer, colorectal cancer, esophageal cancer, endometrial cancer, gastrointestinal cancer, gum cancer, kidney cancer, liver cancer, nasopharynx cancer, prostate cancer, skin cancer, stomach cancer, testis cancer, tongue cancer, melanoma, pancreatic cancer, sarcoma, adenoid cancer or uterine cancer.

40. (canceled)

41. (canceled)

42. The method of claim 24, further comprising administering the composition comprising a N1-methyl-pyrazoloanthrone to the subject if:
a. a biological sample obtained from the subject has increased mRNA or protein expression of at least one member of the Casein Kinase I family or ARK5/NUAK1 kinase; or
b. the subject with cancer is determined to have at one genetic alteration in the ARK3/NUAK1 gene and/or at least one genetic alteration in at least one member of the Casein Kinase 1 family.

43.-46. (canceled)

47. A pharmaceutical composition comprising a N1-methyl-pyrazoloanthrone.

48. The pharmaceutical composition of claim 47, wherein the N1-methyl-pyrazoloanthrone is a compound of formula (I)-(IV), wherein a compound of formula (I) has the following structure: 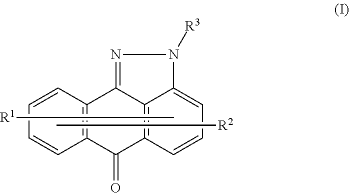
wherein a compound of formula (II): 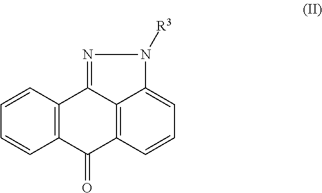
wherein a compound of formula (III) has the following structure: 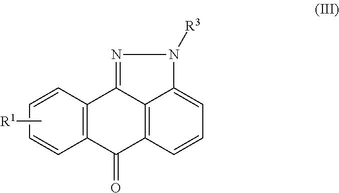
wherein a compound of formula (IV) has the following structure: 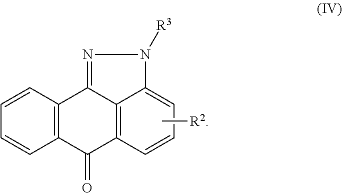
wherein a compound of formula (V) has the following structure: 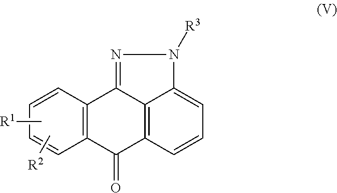
wherein a compound of formula (VI) has the following structure: 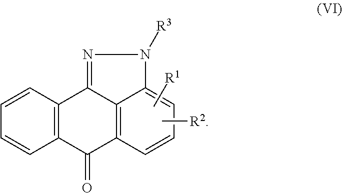
wherein a compound of formula (VII) has the following structure: 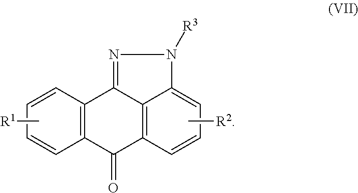
wherein:
R¹ and R² are optional substituents that are the same or different and independently absent, alkyl, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- or di-alkylaminoalkoxy, —N(R⁴)(NR⁵), —NH-alkyl-N(R⁴)(NR), —NHC(O)—R⁶, or —NHSO₂R⁶;
R³ is alkyl, trifluoromethyl, C(O)R⁶, SO₂R⁶, aryl, heteroaryl, cycloalkyl, heterocycloalkyl, arylalkyl, or -alkyl-cycloalkyl;
R⁴ and R⁵ taken together represent alkylidene or a heteroatom-containing alkylidene, or R⁴ and R⁵ are the same or different and independently represent hydrogen, alkyl, cycloalkyl, aryl, arylalkyl, cycloalkylalkyl, aryloxyalkyl, alkoxyalkyl, alkoxyamino, or alkoxy(mono- or di-alkylamino);
R⁶ represents hydrogen, alkyl, cycloalkyl, aryl, arylalkyl, cycloalkylalkyl, alkoxy, amino, mono- or di-alkylamino, arylamino, arylalkylamino, cycloalkylamino, or cycloalkylalkylamino; and
pharmaceutically acceptable salts thereof.

49. The pharmaceutical composition of claim 48, wherein R³ can be a C₁-C₆ alkyl.

50. The pharmaceutical composition of claim 48, wherein the N1-methyl-pyrazoloanthrone is N1-methyl-1,9-pyrazoloanthrone or a functional derivative or a functional analogue thereof.

51. The pharmaceutical composition of claim 47 further comprising a chemotherapeutic agent selected from the group consisting of: cisplatin, doxorubicin, paclitaxel, rapamycin, vermurafib, Mullerian Inhibiting Substance (MIS), recombinant human MIS (rhMIS) or a biologically functional homologue thereof.

52.-82. (canceled)
